SEQUENCE LISTING (105 sequences)

SEQ ID NO: 1 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 3 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 4 - protein, 122 aa, synthetic construct [sequence omitted]

SEQ ID NO: 5 - protein, 107 aa, synthetic construct [sequence omitted]

SEQ ID NO: 6 - protein, 5 aa, synthetic construct
  REGION 1..5: note = antibody HCDR1
SFAMS

SEQ ID NO: 7 - protein, 17 aa, synthetic construct
  REGION 1..17: note = antibody HCDR2
AISGSGGGTYYADSVKG

SEQ ID NO: 8 - protein, 13 aa, synthetic construct
  REGION 1..13: note = antibody HCDR3
DKILWFGEPVFDY

SEQ ID NO: 9 - protein, 11 aa, synthetic construct
  REGION 1..11: note = antibody LCDR1
RASQSVSSYLA

SEQ ID NO: 10 - protein, 7 aa, synthetic construct
  REGION 1..7: note = antibody LCDR2
DASNRAT

SEQ ID NO: 11 - protein, 10 aa, synthetic construct
  REGION 1..10: note = antibody LCDR3
QQRSNWPPTF

SEQ ID NO: 12 - protein, 452 aa, synthetic construct
  REGION 1..452: note = antibody heavy chain
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, synthetic construct
  REGION 1..214: note = antibody light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 120 aa, synthetic construct
  REGION 1..120: note = mAb 003 VH
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSS

SEQ ID NO: 15 - protein, 107 aa, synthetic construct
  REGION 1..107: note = mAb 003 VL
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIK

SEQ ID NO: 16 - protein, 122 aa, synthetic construct
  REGION 1..122: note = mAb 024 VH
EVQLVQSGAEVKKPGESLKISCKGSGYSFSNYWIGWVRQMPGKGLEWMGIIYPHDSDARYSPSFQGQVTFSADKSISTAYLQWSSLKASDTAMYYCARHVGWGSRYWYFDLWGRGTLVTVSS

SEQ ID NO: 17 - protein, 107 aa, synthetic construct
  REGION 1..107: note = mAb 024 VL
EIVLTQSPATLSLSPGERATLSCRASQSVSSYLAWYQQKPGQAPGLLIYDASNRASGIPARFSGSGSGTDFTLTISSLEPEDFAVYYCQQRSNWPLTFGGGTKVEIK

SEQ ID NO: 18 - protein, 120 aa, synthetic construct
  REGION 1..120: note = MOR-202 VH
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 19 - protein, 109 aa, synthetic construct
  REGION 1..109: note = MOR-202 VL
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 20 - protein, 120 aa, synthetic construct [sequence omitted]

SEQ ID NO: 21 - protein, 107 aa, synthetic construct [sequence omitted]

SEQ ID NO: 22 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 23 - protein, 7 aa, synthetic construct
  REGION 1..7: note = BCMB69 HCDR1
SGSYFWG

SEQ ID NO: 24 - protein, 16 aa, synthetic construct
  REGION 1..16: note = BCMB69 HCDR2
SIYYSGITYYNPSLKS

SEQ ID NO: 25 - protein, 11 aa, synthetic construct
  REGION 1..11: note = BCMB69 HCDR3
HDGAVAGLFDY

SEQ ID NO: 26 - protein, 11 aa, synthetic construct
  REGION 1..11: note = BCMB69 LCDR1
GGNNIGSKSVH

SEQ ID NO: 27 - protein, 7 aa, synthetic construct
  REGION 1..7: note = BCMB69 LCDR2
DDSDRPS

SEQ ID NO: 28 - protein, 11 aa, synthetic construct
  REGION 1..11: note = BCMB69 LCDR3
QVWDSSSDHVV

SEQ ID NO: 29 - protein, 121 aa, synthetic construct
  REGION 1..121: note = BCMB69 VH
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSS

SEQ ID NO: 30 - protein, 111 aa, synthetic construct
  REGION 1..111: note = BCMB69 VL
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQP

SEQ ID NO: 31 - protein, 448 aa, synthetic construct
  REGION 1..448: note = BCMB69 HC
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 214 aa, synthetic construct
  REGION 1..214: note = BCMB69 LC
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 33 - protein, 5 aa, synthetic construct
  REGION 1..5: note = CD3B219 HCDR1
TYAMN

SEQ ID NO: 34 - protein, 19 aa, synthetic construct
  REGION 1..19: note = CD3B219 HCDR2
RIRSKYNNYATYYAASVKG

SEQ ID NO: 35 - protein, 14 aa, synthetic construct
  REGION 1..14: note = CD3B219 HCDR3
HGNFGNSYVSWFAY

SEQ ID NO: 36 - protein, 14 aa, synthetic construct
  REGION 1..14: note = CD3B219 LCDR1
RSSTGAVTTSNYAN

SEQ ID NO: 37 - protein, 7 aa, synthetic construct
  REGION 1..7: note = CD3B219 LCDR2
GTNKRAP

SEQ ID NO: 38 - protein, 9 aa, synthetic construct
  REGION 1..9: note = CD3B219 LCDR3
ALWYSNLWV

SEQ ID NO: 39 - protein, 125 aa, synthetic construct
  REGION 1..125: note = CD3B219 VH
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSS

SEQ ID NO: 40 - protein, 112 aa, synthetic construct
  REGION 1..112: note = CD3BB219 VL
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQP

SEQ ID NO: 41 - protein, 452 aa, synthetic construct
  REGION 1..452: note = CD3B219 HC
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 42 - protein, 215 aa, synthetic construct
  REGION 1..215: note = CD3219 LC
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 43 - protein, 5 aa, synthetic construct
  REGION 1..5: note = GC5B596 HCDR1
GYTMN

SEQ ID NO: 44 - protein, 17 aa, synthetic construct
  REGION 1..17: note = GC5B596 HCDR2
LINPYNSDTNYAQKLQG

SEQ ID NO: 45 - protein, 9 aa, synthetic construct
  REGION 1..9: note = GC5B596 HCDR3
VALRVALDY

SEQ ID NO: 46 - protein, 11 aa, synthetic construct
  REGION 1..11: note = GC5B596 LCDR1
KASQNVATHVG

SEQ ID NO: 47 - protein, 7 aa, synthetic construct
  REGION 1..7: note = GC5B596 LCDR2
SASYRYS

SEQ ID NO: 48 - protein, 9 aa, synthetic construct
  REGION 1..9: note = GC5B596 LCDR3
QQYNRYPYT

SEQ ID NO: 49 - protein, 118 aa, synthetic construct
  REGION 1..118: note = GC5B596 VH
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSS

SEQ ID NO: 50 - protein, 107 aa, synthetic construct
  REGION 1..107: note = GC5B596 VL
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIK

SEQ ID NO: 51 - protein, 445 aa, synthetic construct
  REGION 1..445: note = GC5B596 HC
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 52 - protein, 210 aa, synthetic construct
  REGION 1..210: note = GC5B596 LC
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 53 - protein, 500 aa, synthetic construct
  REGION 1..500: note = blinatumomab
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHH

SEQ ID NO: 54 - protein, 5 aa, synthetic construct
  REGION 1..5: note = PSMB127 HCDR1
SDAMH

SEQ ID NO: 55 - protein, 17 aa, synthetic construct
  REGION 1..17: note = -PSMB127 HCDR2
EISGSGGYTNYADSVKG

SEQ ID NO: 56 - protein, 15 aa, synthetic construct
  REGION 1..15: note = PSMB127 HCDR3
DSYDSSLYVGDYFDY

SEQ ID NO: 57 - protein, 378 aa, Homo sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 58 - protein, 556 aa, Homo sapiens [sequence omitted]

SEQ ID NO: 59 - protein, 9 aa, synthetic construct
  REGION 1..9: note = PSMB127 LCDR3
QQRSNWPLT

SEQ ID NO: 60 - protein, 124 aa, synthetic construct
  REGION 1..124: note = PSMB127 VH
EVQLLESGGGLVQPGGSLRLSCAASGFTFKSDAMHWVRQAPGKGLEWVSEISGSGGYTNYADSVKGRFTISRDNSKNTLYLQMNSLRAEDTAVYYCARDSYDSSLYVGDYFDYWGQGTLVTVSS

SEQ ID NO: 61 - protein, 107 aa, synthetic construct
  REGION 1..107: note = PSMB127 VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIK

SEQ ID NO: 62 - protein, 451 aa, synthetic construct
  REGION 1..451: note = PSMB127 HC
EVQLLESGGGLVQPGGSLRLSCAASGFTFKSDAMHWVRQAPGKGLEWVSEISGSGGYTNYADSVKGRFTISRDNSKNTLYLQMNSLRAEDTAVYYCARDSYDSSLYVGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 63 - protein, 214 aa, synthetic construct
  REGION 1..214: note = PSMB127 LC
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 5 aa, synthetic construct
  REGION 1..5: note = TMEB762 HCDR1
SYSMS

SEQ ID NO: 65 - protein, 17 aa, synthetic construct
  REGION 1..17: note = TMEB762 HCDR2
VISGSGGFTDYADSVKG

SEQ ID NO: 66 - protein, 9 aa, synthetic construct
  REGION 1..9: note = TMEB762 HCDR3
MPLNSPHDY

SEQ ID NO: 67 - protein, 11 aa, synthetic construct
  REGION 1..11: note = TMEB762 LCDR1
RASQGIRNDLG

SEQ ID NO: 68 - protein, 7 aa, synthetic construct
  REGION 1..7: note = TMEB762 LCDR2
AASSLQS

SEQ ID NO: 69 - protein, 9 aa, synthetic construct
  REGION 1..9: note = TMEB762 LCDR3
LQDYNYPLT

SEQ ID NO: 70 - protein, 118 aa, synthetic construct
  REGION 1..118: note = TMEB762 VH
EVQLLESGGGLVQPGGSLRLSCAASGFTFSSYSMSWVRQAPGKGLEWVSVISGSGGFTDYADSVKGRFTISRDNSKNTLYLQMNSLRAEDTAVYYCARMPLNSPHDYWGQGTLVTVSS

SEQ ID NO: 71 - protein, 107 aa, synthetic construct
  REGION 1..107: note = TMEB762 VL
DIQMTQSPSSLSASVGDRVTITCRASQGIRNDLGWYQQKPGKAPKLLIYAASSLQSGVPSRFSGSGSGTDFTLTISSLQPEDFATYYCLQDYNYPLTFGGGTKVEIK

SEQ ID NO: 72 - protein, 444 aa, synthetic construct
  REGION 1..444: note = TMEB762 HC
VQLLESGGGLVQPGGSLRLSCAASGFTFSSYSMSWVRQAPGKGLEWVSVISGSGGFTDYADSVKGRFTISRDNSKNTLYLQMNSLRAEDTAVYYCARMPLNSPHDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 73 - protein, 214 aa, synthetic construct
  REGION 1..214: note = TMEB762 LC
DIQMTQSPSSLSASVGDRVTITCRASQGIRNDLGWYQQKPGKAPKLLIYAASSLQSGVPSRFSGSGSGTDFTLTISSLQPEDFATYYCLQDYNYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 7 aa, synthetic construct
  REGION 1..7: note = CD3B396 HCDR1
NNNAAWS

SEQ ID NO: 75 - protein, 18 aa, synthetic construct
  REGION 1..18: note = CD3B396 HCDR2
RTYYRSKWLYDYAVSVKS

SEQ ID NO: 76 - protein, 8 aa, synthetic construct
  REGION 1..8: note = CD3B396 HCDR3
GYSSSFDY

SEQ ID NO: 77 - protein, 14 aa, synthetic construct
  REGION 1..14: note = CD3B396 LCDR1
TGTSSNIGTYKFVS

SEQ ID NO: 78 - protein, 7 aa, synthetic construct
  REGION 1..7: note = CD3B396 LCDR2
EVSKRPS

SEQ ID NO: 79 - protein, 10 aa, synthetic construct
  REGION 1..10: note = CD3B396 LCDR3
VSYAGSGTLL

SEQ ID NO: 80 - protein, 120 aa, synthetic construct
  REGION 1..120: note = CD3B396 VH
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSS

SEQ ID NO: 81 - protein, 110 aa, synthetic construct
  REGION 1..110: note = CD3B396 VL
QSALTQPASVSGSPGQSITISCTGTSSNIGTYKFVSWYQQHPDKAPKVLLYEVSKRPSGVSSRFSGSKSGNTASLTISGLQAEDQADYHCVSYAGSGTLLFGGGTKLTVL

SEQ ID NO: 82 - protein, 447 aa, synthetic construct
  REGION 1..447: note = CD3B396 HC
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 83 - protein, 216 aa, synthetic construct
  REGION 1..216: note = CD3B396 LC
QSALTQPASVSGSPGQSITISCTGTSSNIGTYKFVSWYQQHPDKAPKVLLYEVSKRPSGVSSRFSGSKSGNTASLTISGLQAEDQADYHCVSYAGSGTLLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 84 - protein, 5 aa, synthetic construct
  REGION 1..5: note = C33B904 HCDR1
DYAMH

SEQ ID NO: 85 - protein, 17 aa, synthetic construct
  REGION 1..17: note = C33B904 HCDR2
GIGWSGGSIVYADSVKG

SEQ ID NO: 86 - protein, 10 aa, synthetic construct
  REGION 1..10: note = C33B904 HCDR3
DSPYGDFFDY

SEQ ID NO: 87 - protein, 17 aa, synthetic construct
  REGION 1..17: note = C33B904 LCDR1
KSSQTVFYSSNNKNYLA

SEQ ID NO: 88 - protein, 7 aa, synthetic construct
  REGION 1..7: note = C33B904 LCDR2
WASTRKS

SEQ ID NO: 89 - protein, 9 aa, synthetic construct
  REGION 1..9: note = C33B904 LCDR3
QHYYSTPYT

SEQ ID NO: 90 - protein, 119 aa, synthetic construct
  REGION 1..119: note = -C33B904 VH
EVQLVESGGGLVQPGRSLRLSCAASGFTFDDYAMHWVRQAPGKGLEWVSGIGWSGGSIVYADSVKGRFTISRDNAKNSLYLQMNSLRAEDTALYYCAKDSPYGDFFDYWGQGTLVTVSS

SEQ ID NO: 91 - protein, 113 aa, synthetic construct
  REGION 1..113: note = C33B904 VL
DIVMTQSPDSLAVSLGERATINCKSSQTVFYSSNNKNYLAWYQQKPGQPPKLLISWASTRKSGVPDRFSGSGSGTDFTLTVSSLQAEDVAVYYCQHYYSTPYTFGQGTKLEIK

SEQ ID NO: 92 - protein, 446 aa, synthetic construct
  REGION 1..446: note = C33B904 HC
EVQLVESGGGLVQPGRSLRLSCAASGFTFDDYAMHWVRQAPGKGLEWVSGIGWSGGSIVYADSVKGRFTISRDNAKNSLYLQMNSLRAEDTALYYCAKDSPYGDFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 93 - protein, 216 aa, synthetic construct
  REGION 1..216: note = C33B904 LC
DIVMTQSPDSLAVSLGERATINCKSSQTVFYSSNNKNYLAWYQQKPGQPPKLLISWASTRKSGVPDRFSGSGSGTDFTLTVSSLQAEDVAVYYCQHYYSTPYTFGQGTKLEIKKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 94 - protein, 5 aa, synthetic construct
  REGION 1..5: note = I3RB218 HCDR1
GYWMH

SEQ ID NO: 95 - protein, 17 aa, synthetic construct
  REGION 1..17: note = I3RB218 HCDR2
AIRSDGSSKYYADSVKG

SEQ ID NO: 96 - protein, 10 aa, synthetic construct
  REGION 1..10: note = I3RB218 HCDR3
DGVIEDTFDY

SEQ ID NO: 97 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 98 - protein, 345 aa, Homo sapiens
MYKDCIESTGDYFLLCDAEGPWGIILESLAILGIVVTILLLLAFLFLMRKIQDCSQWNVLPTQLLFLLSVLGLFGLAFAFIIELNQQTAPVRYFLFGVLFALCFSCLLAHASNLVKLVRGCVSFSWTTILCIAIGCSLLQIIIATEYVTLIMTRGMMFVNMTPCQLNVDFVVLLVYVLFLMALTFFVSKATFCGPCENWKQHGRLIFITVLFSIIIWVVWISMLLRGNPQFQRQPQWDDPVVCIALVTNAWVFLLLYIVPELCILYRSCRQECPLQGNACPVTAYQHSFQVENQELSRARDSDGAEEDVALTSYGTPIQPQTVDPTQECFIPQAKLSPQQDAGGV

SEQ ID NO: 99 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 100 - protein, 119 aa, synthetic construct
  REGION 1..119: note = 3RB218 VH
EVQLLESGGGLVQPGGSLRLSCAASGFTFSGYWMHWVRQAPGKGLEWVSAIRSDGSSKYYADSVKGRFTISRDNSKNTLYLQMNSLRAEDTAVYYCAKDGVIEDTFDYWGQGTLVTVSS

SEQ ID NO: 101 - protein, 374 aa, Homo sapiens
MVLWESPRQCSSWTLCEGFCWLLLLPVMLLIVARPVKLAAFPTSLSDCQTPTGWNCSGYDDRENDLFLCDTNTCKFDGECLRIGDTVTCVCQFKCNNDYVPVCGSNGESYQNECYLRQAACKQQSEILVVSEGSCATDAGSGSGDGVHEGSGETSQKETSTCDICQFGAECDEDAEDVWCVCNIDCSQTNFNPLCASDGKSYDNACQIKEASCQKQEKIEVMSLGRCQDNTTTTTKSEDGHYARTDYAENANKLEESAREHHIPCPEHYNGFCMHGKCEHSINMQEPSCRCDAGYTGQHCEKKDYSVLYVVPGPVRFQYVLIAAVIGTIQIAVICVVVLCITRKCPRSNRIHRQKQNTGHYSSDNTTRASTRLI

SEQ ID NO: 102 - protein, 446 aa, synthetic construct
  REGION 1..446: note = I3RB218 HC
EVQLLESGGGLVQPGGSLRLSCAASGFTFSGYWMHWVRQAPGKGLEWVSAIRSDGSSKYYADSVKGRFTISRDNSKNTLYLQMNSLRAEDTAVYYCAKDGVIEDTFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 103 - protein, 330 aa, Homo sapiens [sequence omitted]

SEQ ID NO: 104 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 105 - protein, 509 aa, synthetic construct
  REGION 1..509: note = rHuPH20
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

(19) United States
(12) Patent Application Publication
(10) Pub. No.: US 2024/0101697 A1
(43) Pub. Date: Mar. 28, 2024

(54) METHODS OF TREATING CANCERS AND ENHANCING EFFICACY OF T CELL REDIRECTING THERAPEUTICS

(71) Applicants: Janssen Biotech, Inc., Horsham, PA (US); Stichting VUmc, Amsterdam (NL)

(72) Inventors: Homer Adams, Quakertown, PA (US); Francois Gaudet, Princeton, NJ (US); Niels Van de Donk, De Boelalaan (NL); Kris Frerichs, De Boelalaan (NL); Christie Verkleij, De Boelalaan (NL)

(21) Appl. No.: 18/208,361

(22) Filed: Jun. 12, 2023

Related U.S. Application Data
(62) Division of application No. 16/412,701, filed on May 15, 2019.
(60) Provisional application No. 62/842,080, filed on May 2, 2019.
(60) Provisional application No. 62/736,804, filed on Sep. 26, 2018.
(60) Provisional application No. 62/672,222, filed on May 16, 2018.

(51) Int. Cl. C07K 16/28; A61K 45/06; A61P 35/00; A61P 35/02; C07K 16/30; C07K 16/46

(52) U.S. Cl. CPC C07K 16/2896; A61K 45/06; A61P 35/00; A61P 35/02; C07K 16/2809; C07K 16/2878; C07K 16/30; C07K 16/468; A61K 2039/505; C07K 2317/21; C07K 2317/31

(57) ABSTRACT

Disclosed are methods of treating cancers and enhancing efficacy of T cell redirecting therapeutics.

### CROSS-REFERENCE TO RELATED APPLICATIONS

This application claims the benefit of U.S. Provisional Application Ser. No. 62/672,222, filed 16 May 2018, U.S. Provisional Application Ser. No. 62/736,804, filed 26 Sep. 2018, and U.S. Provisional Application Ser. No. 62/842,080, filed 2 May 2019, the entire contents of which are incorporated herein by reference.

### SEQUENCE LISTING

The instant application contains a Sequence Listing which has been submitted electronically in ASCII format and is hereby incorporated by reference in its entirety. Said ASCII copy, created on May 5, 2019, is named JBI6084USNP1_ST25.txt and is 131 kilobytes in size.

### FIELD OF THE INVENTION

Disclosed are methods of treating cancers and enhancing efficacy of T cell redirecting therapeutics.

### BACKGROUND OF THE INVENTION

T cell redirected killing is a desirable mode of action in many therapeutic areas. In general T cell redirecting molecules are engineered to have at least two antigen binding sites wherein one site binds a surface antigen on a target cell and the other site binds a T cell surface antigen. Amongst T cell surface antigens, the human CD3 epsilon subunit from the TCR protein complex has been the most targeted to redirect T cell killing. Various bispecific antibody formats have been shown to mediate T cell redirection in both in pre-clinical and clinical investigations (May C et al., Biochem Pharmacol, 84: 1105-12, 2012; Frankel S R & Baeuerle P A, Curr Opin Chem Biol, 17(3): 385-92, 2013).

Tumors evade immune recognition through creating an immunosuppressive tumor microenvironment (TME). In the TME, under conditions of persistent antigen and inflammation, T cells become exhausted, or dysfunctional, and progressively lose their effector function and proliferative capacity. Impaired function and number of available T cells to engage therapeutics mediating T cell redirected killing may impair anti-tumor efficacy of the therapeutic. Therefore, there is a need to enhance T cell functionality for optimal efficacy of the therapeutics mediating T cell redirected killing.

### SUMMARY OF THE INVENTION

The disclosure provides a method of treating a cancer in a subject, comprising administering a therapeutically effective amount of an anti-CD38 antibody and a T cell redirecting therapeutic to the subject to treat the cancer.

The disclosure also provides a method of killing a tumor cell in a subject, comprising administering to the subject an anti-CD38 antibody and a T cell redirecting therapeutic that binds an antigen on the tumor cell for a time sufficient to kill the tumor cell.

The disclosure provides a method of enhancing efficacy of a T cell redirecting therapeutic in a subject having a cancer, comprising administering to the subject an anti-CD38 antibody.

The disclosure also provides a method of treating a cancer in a subject, comprising administering a therapeutically effective amount of a BCMAxCD3 bispecific antibody and an anti-CD38 antibody to the subject to treat the cancer.

The disclosure also provides method of treating a cancer in a subject, comprising administering a therapeutically effective amount of a BCMAxCD3 bispecific antibody to the subject to treat the cancer, wherein the subject has been treated with an anti-CD38 antibody prior to administering the BCMAxCD3 bispecific antibody.

The disclosure also provides a method of treating a cancer in a subject, comprising administering a therapeutically effective amount of a BCMAxCD3 bispecific antibody to the subject to treat the cancer, wherein the subject is relapsed or refractory to treatment with a prior anti-cancer therapeutic.

The disclosure also provides a method of treating a multiple myeloma in a subject, comprising administering a therapeutically effective amount of a BCMAxCD3 bispecific antibody and an anti-CD38 antibody to the subject to treat the multiple myeloma.

The disclosure also provides a method of treating a multiple myeloma in a subject, comprising administering a therapeutically effective amount of a BCMAxCD3 bispecific antibody to the subject to treat the multiple myeloma, wherein the subject has been treated with an anti-CD38 antibody prior to administering the BCMAxCD3 bispecific antibody.

The disclosure also provides a method of treating a multiple myeloma in a subject, comprising administering a therapeutically effective amount of a BCMAxCD3 bispecific antibody to the subject to treat the multiple myeloma, wherein the subject is relapsed or refractory to treatment with a prior multiple myeloma therapeutic.

The disclosure also provides a pharmaceutical composition comprising a BCMAxCD3 bispecific antibody comprising a BCMA binding domain comprising a VH of SEQ ID NO: 29 and a VL of SEQ ID NO: 30 and a CD3 binding domain comprising the VH of SEQ ID NO: 39 and the VL of SEQ ID NO: 40, and an anti-CD38 antibody comprising a VH of SEQ ID NO: 4 and the VL of SEQ ID NO: 5.

The disclosure also provides a method of treating a cancer in a subject, comprising administering a therapeutically effective amount of a T-cell redirecting therapeutic that binds GPRC5D and an anti-CD38 antibody to the subject to treat the cancer.

The disclosure also provides a method of treating a cancer in a subject, comprising administering a therapeutically effective amount of a GPRC5DxCD3 bispecific antibody to the subject to treat the cancer, wherein the subject is relapsed or refractory to treatment with a prior anti-cancer therapeutic.

The disclosure also provides a pharmaceutical combination comprising a GPRC5DxCD3 bispecific antibody comprising a GPRC5D binding domain comprising the HCDR1 of SEQ ID NO: 43, the HCDR2 of SEQ ID NO: 44, the HCDR3 of SEQ ID NO: 45, the LCDR1 of SEQ ID NO: 46, the LCDR2 of SEQ ID NO: 47 and the LCDR3 of SEQ ID NO: 48, and a CD3 binding domain comprising the HCDR1 of SEQ ID NO: 33, the HCDR2 of SEQ ID NO: 34, the HCDR3 of SEQ ID NO: 35, the LCDR1 of SEQ ID NO: 36, the LCDR2 of SEQ ID NO: 37 and the LCDR3 of SEQ ID NO: 38 and an anti-CD38 antibody comprising the HCDR1 of SEQ ID NO: 6, the HCDR2 of SEQ ID NO: 7, the HCDR3 of SEQ ID NO: 8, the LCDR1 of SEQ ID NO: 9, the LCDR2 of SEQ ID NO: 10 and the LCDR3 of SEQ ID NO: 11.

The disclosure also provides a method of treating a cancer in a subject, comprising administering a therapeutically effective amount of a T-cell redirecting therapeutic that binds CD19 and an anti-CD38 antibody to the subject to treat the cancer.

The disclosure also provides a method of enhancing efficacy of a T cell redirecting therapeutic that binds CD19 in a subject having a cancer, comprising administering to the subject an anti-CD38 antibody prior to administering the T cell redirecting therapeutic that binds CD19.

The disclosure also provides a pharmaceutical combination comprising a CD19xCD3 bispecific antibody comprising blinatumomab of SEQ ID NO: 53 an anti-CD38 antibody comprising the HCDR1 of SEQ ID NO: 6, the HCDR2 of SEQ ID NO: 7, the HCDR3 of SEQ ID NO: 8, the LCDR1 of SEQ ID NO: 9, the LCDR2 of SEQ ID NO: 10 and the LCDR3 of SEQ ID NO: 11.

The disclosure also provides a kit comprising the pharmaceutical composition of the disclosure.

### BRIEF DESCRIPTION OF THE DRAWINGS

FIG. 1 shows JNJ-957-mediated lysis of multiple myeloma (MM) cell line RPMI8226. Healthy donor peripheral blood mononuclear cells (PB MNCs) were used as effector cells.

FIG. 2 shows JNJ-957-mediated lysis of multiple myeloma (MM) cell line UM9. Healthy donor peripheral blood mononuclear cells (PB MNCs) were used as effector cells.

FIG. 3 shows JNJ-957-mediated lysis of multiple myeloma (MM) cell line U226. Healthy donor peripheral blood mononuclear cells (PB MNCs) were used as effector cells.

FIG. 4 shows JNJ-957-mediated lysis of multiple myeloma (MM) cell line MM1. Healthy donor peripheral blood mononuclear cells (PB MNCs) were used as effector cells.

FIG. 5 shows that, in a representative example (n=2) of RPMI 8226 cells incubated with healthy donor PB MNCs, JNJ-957-mediated MM cell lysis was accompanied by CD4⁺ T cell activation and degranulation as determined by increased surface expression of CD25 (activation).

FIG. 6 shows that, in a representative example (n=2) of RPMI 8226 cells incubated with healthy donor PB MNCs, JNJ-957-mediated MM cell lysis was accompanied by CD4⁺ T cell activation and degranulation as determined by increased surface expression of CD107a (degranulation).

FIG. 7 shows that, in a representative example (n=2) of RPMI 8226 cells incubated with healthy donor PB MNCs, JNJ-957-mediated MM cell lysis was accompanied by CD4⁺ T cell activation and degranulation as determined by the proportion of CD25 and CD107a double positive CD4+ T cells.

FIG. 8 shows that, in a representative example (n=2) of RPMI 8226 cells incubated with healthy donor PB MNCs, JNJ-957-mediated MM cell lysis was accompanied by CD8⁺ T cell activation and degranulation as determined by increased surface expression of CD25 (activation).

FIG. 9 shows that, in a representative example (n=2) of RPMI 8226 cells incubated with healthy donor PB MNCs, JNJ-957-mediated MM cell lysis was accompanied by CD8⁺ T cell activation and degranulation as determined by increased surface expression of CD107a (degranulation);

FIG. 10 shows that, in a representative example (n=2) of RPMI 8226 cells incubated with healthy donor PB MNCs, JNJ-957-mediated MM cell lysis was accompanied by CD8⁺ T cell activation and degranulation as determined by increased proportion of CD25 and CD107a double positive CD4+ T cells.

FIG. 11 shows the in vitro daratumumab-mediated lysis of MM cells from newly diagnosed multiple myeloma (NDMM) and daratumumab naïve relapsed/refractory MM (RRMM) patients. Multiple myeloma cells from daratumumab refractory RRMM patients were resistant to daratumumab-mediated lysis ****P<0.0001

FIG. 12 shows the dose response of JNJ-957-mediated lysis of plasma cells, T cell and NK cells in fully autologous bone marrow (BM) MNCs obtained from newly diagnosed multiple myeloma patients (NDMM, n=8). Percent lysis was measured at various antibody concentrations (0.0064-4.0 μg/mL) as indicated in the Figure. Circles (Top line): plasma cells; Squares (Middle line): T cells; Triangles (Bottom line): NK cells.

FIG. 13 shows the dose response of JNJ-957-mediated lysis of plasma, T cell and NK cells in fully autologous bone marrow (BM) MNCs obtained from multiple myeloma (MM) patients who were refractory to lenalidomide treatment (n=15). Percent lysis was measured at various antibody concentrations (0.0064-4.0 μg/mL) as indicated in the Figure. Circles (Top line): plasma cells; Squares (Middle line): T cells; Triangles (Bottom line): NK cells.

FIG. 14 shows the dose response of JNJ-957-mediated lysis of plasma, T cell and NK cells in fully autologous bone marrow (BM) MNCs obtained from MM patients who were refractory to treatment with lenalidomide and daratumumab (n=11). Percent lysis was measured at various antibody concentrations (0.0064-4.0 μg/mL) as indicated in the Figure. Circles (Top line): plasma cells; Squares (Middle line): T cells; Triangles (Bottom line): NK cells.

FIG. 15 shows that JNJ-957-mediated MM cell lysis was accompanied by activation (as assessed by increased CD25 surface expression) of CD4⁺ T cells in the BM samples from NDMM, daratumumab naïve RRMM (RRMM) and daratumumab refractory RRMM (RRMM daraR) patients. 3930: Isotype control; BC3B4: BCMAxnull bispecific antibody; 7008: nullxCD3 bispecific antibody.

FIG. 16 shows that JNJ-957-mediated MM cell lysis was accompanied by degranulation (as assessed by increased CD107a surface expression) of CD4⁺ T cells in the BM samples from NDMM, daratumumab naïve RRMM (RRMM) and daratumumab refractory RRMM (RRMM daraR) patients. 3930: Isotype control; BC3B4: BCMAxnull bispecific antibody; 7008: nullxCD3 bispecific antibody.

FIG. 17 shows the double positive CD25⁺CD107a⁺ cells as a percentage of CD4⁺ T cells in the BM samples from NDMM, daratumumab naïve RRMM (RRMM) and daratumumab refractory RRMM (RRMM daraR) patients treated with JNJ-957 at indicated concentrations. 3930: Isotype control; BC3B4: BCMAxnull bispecific antibody; 7008: nullxCD3 bispecific antibody. Double positive: CD25 and CD107a double positive CD4⁺ T cells.

FIG. 18 shows that JNJ-957-mediated MM cell lysis was accompanied by activation (as assessed by increased CD25 surface expression) of CD8⁺ T cells in the BM samples from NDMM, daratumumab naïve RRMM (RRMM) and daratumumab refractory RRMM (RRMM daraR) patients. 3930: Isotype control; BC3B4: BCMAxnull bispecific antibody; 7008: nullxCD3 bispecific antibody.

FIG. 19 shows that JNJ-957-mediated MM cell lysis was accompanied by degranulation (as assessed by increased CD107a surface expression) of CD8⁺ T cells in the BM samples from NDMM, daratumumab naïve RRMM (RRMM) and daratumumab refractory RRMM (RRMM daraR) patients. 3930: Isotype control; BC3B4: BCMAxnull bispecific antibody; 7008: nullxCD3 bispecific antibody.

FIG. 20 shows the double positive CD25⁺CD107a⁺ cells as a percentage of CD8⁺ T cells in the BM samples from NDMM, daratumumab naïve RRMM (RRMM) and daratumumab refractory RRMM (RRMM daraR) patients treated with JNJ-957 at indicated concentrations. 3930: Isotype control; BC3B4: BCMAxnull bispecific antibody; 7008: nullxCD3 bispecific antibody. Double positive: CD25 and CD107a double positive CD8⁺ T cells.

FIG. 21 shows BCMA expression levels on MM cells (mean MFI±SEM) in NDMM, daratumumab naïve RRMM and daratumumab refractory RRMM subjects. P-values between the indicated groups were calculated using Mann-Whitney U test; *P<0.05; ns: not significant.

FIG. 22 shows PD-L1 expression levels on MM cells (mean MFI±SEM) in NDMM, daratumumab naïve RRMM and daratumumab refractory RRMM subjects. P-values between the indicated groups were calculated using Mann-Whitney U test; *P<0.05; ns: not significant.

FIG. 23 shows the baseline percentage of Tregs in BM MNCs from NDMM, daratumumab naïve RRMM and daratumumab refractory RRMM. **p<0.01; ns: not significant.

FIG. 24 shows the baseline percentage of activated T cells (as assessed by HLA-DR positivity) in BM MNCs from NDMM, daratumumab naïve RRMM and daratumumab refractory RRMM. **p<0.01; ns: not significant.

FIG. 25 shows the baseline percentage of the various T cell subsets in BM MNCs from NDMM, daratumumab naïve RRMM and daratumumab refractory RRMM. *p<0.05; **p<0.01; Ns: not significant. TEMRA: CD45RA⁺CCR7− T cells; EM: effector memory CM: central memory; N: naïve T cells.

FIG. 26 shows JNJ-957-mediated lysis of multiple myeloma cells from NDMM patients mediated by autologous BM MNCs. Samples were dichotomized for the frequency of Tregs at baseline (low ≤50^{th} percentile, high >50^{th} percentile). Ns: not significant.

FIG. 27 shows JNJ-957-mediated lysis of multiple myeloma cells from daratumumab naïve RRMM patients mediated by autologous BM MNCs. Samples were dichotomized for the frequency of Tregs at baseline (low ≤50^{th} percentile, high >50^{th} percentile). *p<0.05; **p<0.01; Ns: not significant.

FIG. 28 shows JNJ-957-mediated lysis of multiple myeloma cells from daratumumab refractory RRMM patients mediated by autologous BM MNCs. Samples were dichotomized for the frequency of Tregs at baseline (low ≤50^{th} percentile, high >50^{th} percentile). *p<0.05; ns: not significant.

FIG. 29 shows JNJ-957-mediated lysis of MM cells from BM samples from NDMM (n=9), daratumumab naïve RRMM (n=18) and daratumumab-refractory RRMM (n=13) patients after a 48-hour incubation. Data was depicted as mean±SEM, P values were calculated using student t-test. **P<0.01

FIG. 30 shows that JNJ-957-mediated lysis of MM cells from bone marrow (BM) samples obtained from relapsed/refractory multiple myeloma patients (RRMM) (n=8) was augmented in samples from patients who had received daratumumab (“Dara exposed”) when compared to samples from the same patients before initiation of daratumumab treatment (“Dara naïve”). Data was depicted as mean±SEM; P values were calculated using a paired t-test. ns: not significant; *P<0.05, **P<0.01.

FIG. 31 shows the percentage of Tregs in the sequential BM aspirates from RRMM patients before initiation of daratumumab (before dara) and at development of daratumumab refractory disease (dara exposed). ns: not significant.

FIG. 32 shows the percentage of CD4⁺ cells in the sequential BM aspirates from RRMM patients before initiation of daratumumab (before dara) and at development of daratumumab refractory disease (dara exposed). ns: not significant.

FIG. 33 shows the percentage of CD8⁺ T cells in the sequential BM aspirates from RRMM patients before initiation of daratumumab (before dara) and at development of daratumumab refractory disease (dara exposed).

FIG. 34 shows that JNJ-957-mediated lysis of RPMI8226 multiple myeloma cells using patient derived PB MNCs as effector cells was augmented by PB MNCs from patients who had received daratumumab (“PBMNCs during dara”) when compared to samples from the same patients before initiation of daratumumab treatment (“PBMNCs dara naïve”) (n=5). Data was depicted as mean±SEM; P values were calculated using a paired t-test. ns: not significant; *P<0.05.

FIG. 35 shows the percentage of Tregs in PB-MNC samples from daratumumab naïve (before dara) and daratumumab refractory (during dara) RRMM patients.

FIG. 36 shows the percentage of CD4⁺ T cells in PB-MNC samples from daratumumab naïve (before dara) and daratumumab refractory (during dara) RRMM patients. ns: not significant.

FIG. 37 shows the percentage of CD8⁺ T cells in PB-MNC samples from daratumumab naïve (before dara) and daratumumab refractory (during dara) RRMM patients. ns: not significant.

FIG. 38 shows that the addition of daratumumab augmented JNJ-957-mediated MM cell lysis. BM mononuclear cells (MNC) from NDMM (n=8) patients were treated with JNJ-957 (0.032-0.8 μg/mL) alone or in combination with 10 μg/mL daratumumab for 48 hours. The observed (Obs) lysis levels of MM cells by JNJ-957 and daratumumab were compared to the expected (Exp) lysis levels, which were calculated with the assumption that the combinatorial effect is achieved by additive effects as indicated in methods. Black bars depict the group mean value±SEM. P values were calculated using a paired student t-test. ns: not significant.

FIG. 39 shows that the addition of daratumumab augmented JNJ-957-mediated MM cell lysis. BM MNC of daratumumab naïve RRMM (n=17) patients were treated with JNJ-957 (0.032-0.8 μg/mL) alone or in combination with 10 μg/mL daratumumab for 48 hours. The observed (Obs) lysis levels of MM cells by JNJ-957 and daratumumab were compared to the expected (Exp) lysis levels, which were calculated with the assumption that the combinatorial effect is achieved by additive effects as indicated in methods. Black bars depict the group mean value±SEM. P values were calculated using a paired student t-test. ns: not significant.

FIG. 40 shows that the addition of daratumumab augmented JNJ-957-mediated MM cell lysis. BM MNC of daratumumab refractory RRMM (n=14) patients were treated with JNJ-957 (0.032-0.8 μg/mL) alone or in combination with 10 μg/mL daratumumab for 48 hours. The observed (O) lysis levels of MM cells by JNJ-957 and daratumumab were compared to the expected (E) lysis levels, which were calculated with the assumption that the combinatorial effect is achieved by additive effects as indicated in methods. Black bars depict the group mean value±SEM. P values were calculated using a paired student t-test. JNJ-957 is referred to as JNJ-7957 in the Figure. Dara: daratumumab. ns: not significant.

FIG. 41 shows blinatumomab-mediated lysis of the Raji cell line, using sequential PB samples from 11 RRMM patients as effector cells (E:T of 10:1), which were obtained directly before initiation of daratumumab treatment (black, bottom lin) and during daratumumab treatment (grey, top line); median duration of treatment 7 months, range 2-14 months. Blinatumomoab-based cytotoxicity assay was performed after a 48-hour incubation of Raji cells with blinatumomab (0.01-10 μg/mL) in the presence of these PB-MNCs. Data represents mean±SEM, experiments were performed in duplicate. The statistical significance (P-value) between the indicated groups was calculated using nonlinear regression analysis.

FIG. 42 shows a dose response of JNJ-957-mediated lysis of plasma, T cells and NK cells of BM-MNC cells obtained from six primary plasma cell leukemia (pPCL) patients. Percent lysis was measured at various antibody concentrations (0.0064-4.0 μg/mL) as indicated in the Figure. Top line: plasma cells; bottom line: overlapping line for T cells and NK cells. JNJ-957 is refereed to as JNJ-7957 in the Figure.

FIG. 43 shows anti-GPRC5DxCD3 antibody-mediated lysis of the MM cell line, using sequential PB samples from 11 RRMM patients as effector cells (E:T of 10:1), which were obtained directly before initiation of daratumumab treatment (bottom line) and during daratumumab treatment (top line); median duration of treatment 7 months, range 2-14 months. Blinatumomab-based cytotoxicity assay was performed after a 48-hour incubation of Raji cells with blinatumomab (0.01-10 μg/mL) in the presence of these PB-MNCs. Data represents mean±SEM, experiments were performed in duplicate.

FIG. 44 shows that the addition of daratumumab was additive to the anti-GPRC5DxCD3 bispecific antibody (JNJ-7564)-mediated MM cell lysis. BM MNC of daratumumab naïve RRMM (n=17) patients were treated with the anti-GPRC5DxCD3 bispecific antibody (0.00128-0.8 μg/mL) alone or in combination with 0.1 μg/mL daratumumab for 48 hours. The observed (O) lysis levels of MM cells by the anti-GPRC5DxCD3 bispecific antibody and daratumumab were compared to the expected (E) lysis levels, which were calculated with the assumption that the combinatorial effect is achieved by additive effects as indicated in methods. Black bars depict the group mean value±SEM. P values were calculated using a paired student t-test. ns: not significant. Dara: daratumumab.

### DETAILED DESCRIPTION OF THE INVENTION

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods. All patents, published patent applications and publications cited herein are incorporated by reference as if set forth fully herein.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

“About” when used in reference to numerical ranges, cutoffs, or specific values means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of an assay, result or embodiment, “about” means within one standard deviation per the practice in the art, or a range of up to 5%, whichever is larger.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

“Antigen binding fragment” or “antigen binding domain” refers to a portion of an immunoglobulin molecule that binds an antigen. Antigen binding fragments may be synthetic, enzymatically obtainable or genetically engineered polypeptides and include the VH, the VL, the VH and the VL, Fab, F(ab′)2, Fd and Fv fragments, domain antibodies (dAb) consisting of one VH domain or one VL domain, shark variable IgNAR domains, camelized VH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3. VH and VL domains may be linked together via a synthetic linker to form various types of single chain antibody designs where the VH/VL domains may pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804 and WO1992/01047.

“BCMA” refers to human B-cell maturation antigen, also known as CD269 or TNFRSF17 (UniProt Q02223). The extracellular domain of BCMA encompasses residues 1-54 of Q02223. Human BCMA comprises the amino acid sequence of SEQ ID NO: 2.

SEQ ID NO: 2
MLQMAGQCSQNEYFDSLLHACIPCQLRCSSNTPPLTCQRYCNASVTNSVK
GTNAILWTCLGLSLIISLAVFVLMFLLRKINSEPLKDEFKNTGSGLLGMA
NIDLEKSRTGDEIILPRGLEYTVEECTCEDCIKSKPKVDSDHCFPLPAME
EGATILVTTKTNDYCKSLPAALSATEIEKS

“Bispecific” refers to an antibody that specifically binds two distinct antigens or two distinct epitopes within the same antigen. The bispecific antibody may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor.

“CD123” refers to human Interleukin-3 receptor subunit alpha (IR3RA) having the amino acid sequence shown in SEQ ID NO: 57. The extracellular domain or CD123 spans residues 19-305 of SEQ ID NO: 57.

CD123
(SEQ ID NO: 57)
MVLLWLTLLLIALPCLLQTKEDPNPPITNLRMKAKAQQLTWDLNRNVTDI
ECVKDADYSMPAVNNSYCQFGAISLCEVTNYTVRVANPPFSTWILFPENS
GKPWAGAENLTCWIHDVDFLSCSWAVGPGAPADVQYDLYLNVANRRQQYE
CLHYKTDAQGTRIGCRFDDISRLSSGSQSSHILVRGRSAAFGIPCTDKFV
VFSQIEILTPPNMTAKCNKTHSFMHWKMRSHFNRKFRYELQIQKRMQPVI
TEQVRDRTSFQLLNPGTYTVQIRARERVYEFLSAWSTPQRFECDQEEGAN
TRAWRTSLLIALGTLLALVCVFVICRRYLVMQRLFPRIPHMKDPIGDSFQ
NDKLVVWEAGKAGLEECLVTEVQVVQKT

“CD19” refers to human B-lymphocyte antigen CD19 having the amino acid sequence of SEQ ID NO: 58. The extracellular domain of CD19 spans residues 20-291 of SEQ ID NO: 58.

CD19
(SEQ ID NO: 58)
MPPPRLLFFLLFLTPMEVRPEEPLVVKVEEGDNAVLQCLKGTSDGPTQQL
TWSRESPLKPFLKLSLGLPGLGIHMRPLAIWLFIFNVSQQMGGFYLCQPG
PPSEKAWQPGWTVNVEGSGELFRWNVSDLGGLGCGLKNRSSEGPSSPSGK
LMSPKLYVWAKDRPEIWEGEPPCLPPRDSLNQSLSQDLTMAPGSTLWLSC
GVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYL
IFCLCSLVGILHLQRALVLRRKRKRMTDPTRRFFKVTPPPGSGPQNQYGN
VLSLPTPTSGLGRAQRWAAGLGGTAPSYGNPSSDVQADGALGSRSPPGVG
PEEEEGEGYEEPDSEEDSEFYENDSNLGQDQLSQDGSGYENPEDEPLGPE
DEDSFSNAESYENEDEELTQPVARTMDFLSPHGSAWDPSREATSLGSQSY
EDMRGILYAAPQLRSIRGQPGPNHEEDADSYENMDNPDGPDPAWGGGGRM
GTWSTR

“CD3” refers to a human antigen which is expressed on T cells as part of the multimolecular T cell receptor (TCR) complex and which consists of a homodimer or heterodimer formed from the association of two or four receptor chains: CD3 epsilon, CD3 delta, CD3 zeta and CD3 gamma. Human CD3 epsilon comprises the amino acid sequence of SEQ ID NO: 3. SEQ ID NO: 22 shows the extracellular domain of CD3 epsilon.

SEQ ID NO: 3
MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKNRKAKAKPVTRGAGAGGRQRGQNKERPPPVPNPDYEPIRKGQRDLYS
GLNQRRI
SEQ ID NO: 22
DGNEEMGGITQTPYKVSISGTTVILTCPQYPGSEILWQHNDKNIGGDEDD
KNIGSDEDHLSLKEFSELEQSGYYVCYPRGSKPEDANFYLYLRARVCENC
MEMD

“CD33” refers to myeloid cell surface antigen CD33 having the amino acid sequence of SEQ ID NO: 97. The extracellular domain of CD33 spans residues 18-259 of SEQ ID NO: 97.

CD33
(SEQ ID NO: 97)
MPLLLLLPLLWAGALAMDPNFWLQVQESVTVQEGLCVLVPCTFFHPIPYY
DKNSPVHGYWFREGAIISRDSPVATNKLDQEVQEETQGRFRLLGDPSRNN
CSLSIVDARRRDNGSYFFRMERGSTKYSYKSPQLSVHVTDLTHRPKILIP
GTLEPGHSKNLTCSVSWACEQGTPPIFSWLSAAPTSLGPRTTHSSVLIIT
PRPQDHGTNLTCQVKFAGAGVTTERTIQLNVTYVPQNPTTGIFPGDGSGK
QETRAGVVHGAIGGAGVTALLALCLCLIFFIVKTHRRKAARTAVGRNDTH
PTTGSASPKHQKKSKLHGPTETSSCSGAAPTVEMDEELHYASLNFHGMNP
SKDTSTEYSEVRTQ

“CD38” refers to the human CD38 protein (UniProt accession no. P28907) (synonyms: ADP-ribosyl cyclase 1, cADPr hydrolase 1, cyclic ADP-ribose hydrolase 1). Human CD38 has an amino acid sequence as shown in SEQ ID NO: 1. CD38 is a single pass type II transmembrane protein with amino acid residues 1-21 representing the cytosolic domain, amino acid residues 22-42 representing the transmembrane domain, and residues 43-300 representing the extracellular domain.

SEQ ID NO: 1
MANCEFSPVSGDKPCCRLSRRAQLCLGVSILVLILVVVLAVVVPRWRQQW
SGPGTTKRFPETVLARCVKYTEIHPEMRHVDCQSVWDAFKGAFISKHPCN
ITEEDYQPLMKLGTQTVPCNKILLWSRIKDLAHQFTQVQRDMFTLEDTLL
GYLADDLTWCGEFNTSKINYQSCPDWRKDCSNNPVSVFWKTVSRRFAEAA
CDVVHVMLNGSRSKIFDKNSTFGSVEVHNLQPEKVQTLEAWVIHGGREDS
RDLCQDPTIKELESIISKRNIQFSCKNIYRPDKFLQCVKNPEDSSCTSEI

“CH3 region” or “CH3 domain” refers to the CH3 region of an immunoglobulin. The CH3 region of human IgG1 antibody corresponds to amino acid residues 341-446. However, the CH3 region may also be any of the other antibody isotypes as described herein.

“Chimeric antigen receptor” or “CAR” refers to engineered T cell receptors which graft a ligand or antigen specificity onto T cells (for example naïve T cells central memory T cells effector memory T cells or combinations thereof). CARs are also known as artificial T-cell receptors, chimeric T-cell receptors or chimeric immunoreceptors. CARs comprise an extracellular domain capable of binding to an antigen, a transmembrane domain and at least one intracellular domain. CAR intracellular domain comprises a polypeptide known to function as a domain that transmits a signal to cause activation or inhibition of a biological process in a cell. The transmembrane domain comprises any peptide or polypeptide known to span the cell membrane and that can function to link the extracellular and signaling domains. A chimeric antigen receptor may optionally comprise a hinge domain which serves as a linker between the extracellular and transmembrane domains.

“Combination” means that two or more therapeutics are administered to a subject together in a mixture, concurrently as single agents or sequentially as single agents in any order.

“Complementarity determining regions” (CDR) are antibody regions that bind an antigen. CDRs may be defined using various delineations such as Kabat (Wu et al. J Exp Med 132: 211-50, 1970) (Kabat et al., Sequences of Proteins of Immunological Interest, 5th Ed. Public Health Service, National Institutes of Health, Bethesda, Md., 1991), Chothia (Chothia et al. J Mol Biol 196: 901-17, 1987), IMGT (Lefranc et al. Dev Comp Immunol 27: 55-77, 2003) and AbM (Martin and Thornton J Bmol Biol 263: 800-15, 1996). The correspondence between the various delineations and variable region numbering are described (see e.g. Lefranc et al. Dev Comp Immunol 27: 55-77, 2003; Honegger and Pluckthun, J Mol Biol 309:657-70, 2001; International ImMunoGeneTics (IMGT) database; Web resources, http://www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification

“Comprising” is intended to include examples encompassed by the terms “consisting essentially of” and “consisting of”; similarly, the term “consisting essentially of” is intended to include examples encompassed by the term “consisting of.” Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

“Enhance” or “enhanced” refers to enhancement in one or more functions of a test molecule when compared to a control molecule or a combination of test molecules when compared to one or more control molecules. Exemplary functions that can be measured are tumor cell killing, T cell activation, relative or absolute T cell number, Fc-mediated effector function (e.g. ADCC, CDC and/or ADCP) or binding to an Fcγ receptor (FcγR) or FcRn. “Enhanced” may be an enhancement of about 10%, 20%, 30%, 40%, 50%, 60%, 70%, 80%, 90%, 100% or more, or a statistically significant enhancement.

“Fc gamma receptor” (FcγR) refers to well-known FcγRI, FcγRIIa, FcγRIIb or FcγRIII. Activating FcγR includes FcγRI, FcγRIIa and FcγRIII.

“GPRC5D” refers to human G-protein coupled receptor family C group 5 member D having the amino acid sequence shown in SEQ ID NO: 98.

GPRC5D
(SEQ ID NO: 98)
MYKDCIESTGDYFLLCDAEGPWGIILESLAILGIVVTILLLLAFLFLMRK
IQDCSQWNVLPTQLLFLLSVLGLFGLAFAFIIELNQQTAPVRYFLFGVLF
ALCFSCLLAHASNLVKLVRGCVSFSWTTILCIAIGCSLLQIIIATEYVTL
IMTRGMMFVNMTPCQLNVDFVVLLVYVLFLMALTFFVSKATFCGPCENWK
QHGRLIFITVLFSIIIWVVWISMLLRGNPQFQRQPQWDDPVVCIALVTNA
WVFLLLYIVPELCILYRSCRQECPLQGNACPVTAYQHSFQVENQELSRAR
DSDGAEEDVALTSYGTPIQPQTVDPTQECFIPQAKLSPQQDAGGV

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody may include substitutions in the frameworks so that the frameworks may not be exact copies of expressed human immunoglobulin or human immunoglobulin germline gene sequences.

“Isolated” refers to a homogenous population of molecules (such as synthetic polynucleotides or a protein such as an antibody) which have been substantially separated and/or purified away from other components of the system the molecules are produced in, such as a recombinant cell, as well as a protein that has been subjected to at least one purification or isolation step. “Isolated antibody” refers to an antibody that is substantially free of other cellular material and/or chemicals and encompasses antibodies that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

“Mutation” refers to an engineered or naturally occurring alteration in a polypeptide or polynucleotide sequence when compared to a reference sequence. The alteration may be a substitution, insertion or deletion of one or more amino acids or polynucleotides.

“Non-fixed combination” refers to separate pharmaceutical compositions of the T cell redirecting therapeutic and the anti-CD38 antibody administered as separate entities either simultaneously, concurrently or sequentially with no specific intervening time limits, wherein such administration provides effective levels of the two compounds in the body of the subject.

“Multispecific” refers to an antibody that specifically binds at least two distinct antigens or at least two distinct epitopes within the same antigen. Multispecific antibody may bind for example two, three, four or five distinct antigens or distinct epitopes within the same antigen.

“Pharmaceutical composition” refers to composition that comprises an active ingredient and a pharmaceutically acceptable carrier.

“Pharmaceutically acceptable carrier” or “excipient” refers to an ingredient in a pharmaceutical composition, other than the active ingredient, which is nontoxic to a subject.

“Philadelphia chromosome” or “Ph” refers to a well-known chromosomal translocation between chromosomes 9 and 22, resulting in the oncogenic BCR-ABL gene fusion with constitutively active tyrosine kinase activity. The translocation results in a portion of the BCR gene from chromosome 22q11 becoming fused with a portion of the ABL gene from chromosome 9q34, and is designated as t(9; 22)(q34; q11) under the International System for Human Cytogenetic Nomenclature (ISCN). Depending on the precise location of the fusion, the molecular weight of the resulting fusion protein can range from 185 to 210 kDa. “Philadelphia chromosome” refers to all BCR-ABL fusion proteins formed due the (9; 22)(q34; q11) translocation.

“PSMA” refers to human prostate specific membrane antigen having the amino acid sequence of SEQ ID NO: 99. The extracellular domain spans residues 44-750 of SEQ ID NO: 99.

PSMA
(SEQ ID NO: 99)
MWNLLHETDSAVATARRPRWLCAGALVLAGGFFLLGFLFGWFIKSSNEAT
NITPKHNMKAFLDELKAENIKKFLYNFTQIPHLAGTEQNFQLAKQIQSQW
KEFGLDSVELAHYDVLLSYPNKTHPNYISIINEDGNEIFNTSLFEPPPPG
YENVSDIVPPFSAFSPQGMPEGDLVYVNYARTEDFFKLERDMKINCSGKI
VIARYGKVFRGNKVKNAQLAGAKGVILYSDPADYFAPGVKSYPDGWNLPG
GGVQRGNILNLNGAGDPLTPGYPANEYAYRRGIAEAVGLPSIPVHPIGYY
DAQKLLEKMGGSAPPDSSWRGSLKVPYNVGPGFTGNFSTQKVKMHIHSTN
EVTRIYNVIGTLRGAVEPDRYVILGGHRDSWVFGGIDPQSGAAVVHEIVR
SFGTLKKEGWRPRRTILFASWDAEEFGLLGSTEWAEENSRLLQERGVAYI
NADSSIEGNYTLRVDCTPLMYSLVHNLTKELKSPDEGFEGKSLYESWTKK
SPSPEFSGMPRISKLGSGNDFEVFFQRLGIASGRARYTKNWETNKFSGYP
LYHSVYETYELVEKFYDPMFKYHLTVAQVRGGMVFELANSIVLPFDCRDY
AVVLRKYADKIYSISMKHPQEMKTYSVSFDSLFSAVKNFTEIASKFSERL
QDFDKSNPIVLRMMNDQLMFLERAFIDPLGLPDRPFYRHVIYAPSSHNKY
AGESFPGIYDALFDIESKVDPSKAWGEVKRQIYVAAFTVQAAAETLSEVA

“Recombinant” refers to DNA, antibodies and other proteins that are prepared, expressed, created or isolated by recombinant means when segments from different sources are joined to produce recombinant DNA, antibodies or proteins.

“Reduce” or “reduced” refers to a reduction in one or more functions of a test molecule when compared to a control molecule or a combination of test molecules when compared to one or more control molecules. Exemplary functions that can be measured are tumor cell killing, T cell activation, relative or absolute T cell number, Fc-mediated effector function (e.g. ADCC, CDC and/or ADCP) or binding to an Fcγ receptor (FcγR) or FcRn. “Reduced” may be a reduction of about 10%, 20%, 30%, 40%, 50%, 60%, 70%, 80%, 90%, 100% or more, or a statistically significant enhancement.

“rHuPh20” refers to recombinant human hyalurodinase having the amino acid sequence of SEQ ID NO: 105, which is a recombinant hyaluronidase (HYLENEX® recombinant) described in Int'l Pat. Pub. No. WO2004/078140.

rHuPH20
(SEQ ID NO: 105)
MGVLKFKHIFFRSFVKSSGVSQIVFTFLLIPCCLTLNFRAPPVIPNVPFL
WAWNAPSEFCLGKFDEPLDMSLFSFIGSPRINATGQGVTIFYVDRLGYYP
YIDSITGVTVNGGIPQKISLQDHLDKAKKDITFYMPVDNLGMAVIDWEEW
RPTWARNWKPKDVYKNRSIELVQQQNVQLSLTEATEKAKQEFEKAGKDFL
VETIKLGKLLRPNHLWGYYLFPDCYNHHYKKPGYNGSCFNVEIKRNDDLS
WLWNESTALYPSIYLNTQQSPVAATLYVRNRVREAIRVSKIPDAKSPLPV
FAYTRIVFTDQVLKFLSQDELVYTFGETVALGASGIVIWGTLSIMRSMKS
CLLLDNYMETILNPYIINVTLAAKMCSQVLCQEQGVCIRKNWNSSDYLHL
NPDNFAIQLEKGGKFTVRGKPTLEDLEQFSEKFYCSCYSTLSCKEKADVK
DTDAVDVCIADGVCIDAFLKPPMETEEPQIFYNASPSTLSATMFIVSILF
LIISSVASL

“Refractory” refers to a cancer that is not amendable to surgical intervention and is initially unresponsive to therapy.

“Relapsed” refers to a cancer that responded to treatment but then returns.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. Except when noted, the terms “patient” or “subject” are used interchangeably.

“T cell redirecting therapeutic” refers to a molecule containing two or more binding regions, wherein one of the binding regions specifically binds a cell surface antigen (such as a tumor associated antigen) on a target cell or tissue and wherein a second binding region of the molecule specifically binds a T cell antigen (such as, CD3). This dual/multi-target binding ability recruit T cells to the target cell or tissue leading to the eradication of the target cell or tissue.

“TMEFF2” refers to human transmembrane protein with EGF like and two follistatin like domains 2, also called tomoregulin 2. The amino acid sequence of the full length human TMEFF2 is shown in SEQ ID NO: 101. The extracellular domain of TMEFF2 spans residues 40-374 of SEQ ID NO: 101

TMEFF2
(SEQ ID NO: 101)
MVLWESPRQCSSWTLCEGFCWLLLLPVMLLIVARPVKLAAFPTSLSDCQT
PTGWNCSGYDDRENDLFLCDTNTCKFDGECLRIGDTVTCVCQFKCNNDYV
PVCGSNGESYQNECYLRQAACKQQSEILVVSEGSCATDAGSGSGDGVHEG
SGETSQKETSTCDICQFGAECDEDAEDVWCVCNIDCSQTNFNPLCASDGK
SYDNACQIKEASCQKQEKIEVMSLGRCQDNTTTTTKSEDGHYARTDYAEN
ANKLEESAREHHIPCPEHYNGFCMHGKCEHSINMQEPSCRCDAGYTGQHC
EKKDYSVLYVVPGPVRFQYVLIAAVIGTIQIAVICVVVLCITRKCPRSNR
IHRQKQNTGHYSSDNTTRASTRLI

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

“Treat” or “treatment” refers to both therapeutic treatment and prophylactic or preventative measures, wherein the object is to prevent or slow down (lessen) an undesired physiological change or disorder. Beneficial or desired clinical results include alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if a subject was not receiving treatment. Those in need of treatment include those already with the condition or disorder as well as those prone to have the condition or disorder or those in which the condition or disorder is to be prevented.

“Tumor cell” or a “cancer cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation may arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, MD. (1991), unless otherwise explicitly stated. Antibody constant chain numbering can be found for example at ImMunoGeneTics website, at IMGT Web resources at IMGT Scientific charts.

The substitutions in the CH3 region are expressed as modified position(s) in the first CH3 domain of the first heavy chain/modified position(s) in the second CH3 domain of the second heavy chain. For example, F405L/K409R refers to a F405L mutation in the first CH3 region and K09R mutation in the second CH3 region. L351Y_F405A_Y407V/T394W refers to L351Y, F40FA and Y407V mutations in the first CH3 region and T394W mutation in the second CH3 region. D399FHKRQ/K409AGRH refers to mutation in which D399 may be replaced by F, H, K R or Q, and K409 may be replaced by A, G, R or H.

Conventional one and three-letter amino acid codes are used herein as shown in Table 1.

TABLE 1

| Amino acid | Three-letter code | One-letter code |
|---|---|---|
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Combinations of Anti-CD38 Antibodies and T Cell Redirecting Therapeutics and their Uses

The invention is based, at least in part, on the finding that therapeutic agents JNJ-957 or a GPRC5DxCD3 antibody and the anti-CD38 antibody DARZALEX® (daratumumab), each of which mediate killing of multiple myeloma cells upon target engagement on the same cell did not antagonize each other in terms of competing to bind to or mechanism of action on MM cells or reciprocal downregulation of targets, and therefore are suitable to be used as a combination therapy. The invention is also based, at least in part, on the finding that prior treatment with DARZALEX® (daratumumab) augmented JNJ-957-mediated killing of multiple myeloma cells obtained from heavily treated relapsed/refractory multiple myeloma subjects. The invention is also based, at least in part, on the finding that DARZALEX® (daratumumab) augmented killing of tumor cells other than multiple myeloma cells by T cell redirecting therapeutics targeting non-multiple myeloma tumor cells. Hence combination of anti-CD38 antibodies with T cell redirecting therapeutics and/or pretreatment of subjects with anti-CD38 antibodies prior to administering T cell redirecting therapeutics can enhance anti-tumor efficacy of the monotherapies. Also given that cancers are typically heterogeneous diseases, portions of the cancer may exclusively have sufficient expression of one target vs. the other where combination therapy will aid deeper eradication of the disease.

CD38 is a multifunctional protein having function in receptor-mediated adhesion and signaling as well as mediating calcium mobilization via its ecto-enzymatic activity, catalyzing formation of cyclic ADP-ribose (cADPR) and ADPR. CD38 mediates cytokine secretion and activation and proliferation of lymphocytes (Funaro et al., J Immunol 145:2390-6, 1990; Terhorst et al., Cell 771-80, 1981; Guse et al., Nature 398:70-3, 1999). CD38, via its NAD glycohydrolase activity, also regulates extracellular NAD⁺ levels, which have been implicated in modulating the regulatory T-cell compartment (Adriouch et al., Microbes infect 14:1284-92, 2012; Chiarugi et al., Nature Reviews 12:741-52, 2012). In addition to signaling via Ca²⁺, CD38 signaling occurs via cross-talk with antigen-receptor complexes on T- and B-cells or other types of receptor complexes, e.g., MHC molecules, involving CD38 in several cellular responses, but also in switching and secretion of IgG1. It has been identified herein that an anti-CD38 antibody DARZALEX® (daratumumab) enhances the anti-tumor effect of T cell redirection therapeutics. While not wishing to be bound by any particular theory, it can be hypothesized that DARZALEX® (daratumumab) via its immunomodulatory activity in human subjects (i.e. reducing the number of immune suppressive Tregs, MDSCs and Bregs, increasing the number of CD8⁺ T cells and the ratio of CD8⁺ to Tregs, promoting CD8⁺ central memory cell formation and increasing T cell clonality) may result in enhanced immune responses even in a subjects and therefore may facilitate T cell engagement of T cell redirecting therapeutics.

The disclosure provides a method of treating a cancer in a subject, comprising administering a therapeutically effective amount of an anti-CD38 antibody and a T cell redirecting therapeutic to the subject to treat the cancer.

The disclosure also provides a method of killing a tumor cell in a subject, comprising administering to the subject an anti-CD38 antibody and a T cell redirecting therapeutic that binds an antigen on the tumor cell for a time sufficient to kill the tumor cell.

The disclosure also provides a method of enhancing efficacy of a T cell redirecting therapeutic in a subject having a cancer, comprising administering to the subject an anti-CD38 antibody.

In some embodiments, the anti-CD38 antibody is administered prior to administering the T cell redirecting therapeutic.

The T cell redirecting therapeutic may be administered one day, two days, three days, four days, five days, six days, one week, two weeks, three weeks, one month, five weeks, six weeks, seven weeks, two months, three months, four months, five months, six months or longer prior to administering the anti-CD38 antibody.

In some embodiments, the T cell redirecting therapeutic binds an antigen on a tumor cell.

In some embodiments, the antigen on the tumor cell is BCMA, GPRC5D, CD33, CD123, CD19, PSMA, TMEFF2, CD20, CD10, CD21, CD22, CD25, CD30, CD34, CD37, CD44v6, CD45, CD52, CD133, ROR1, B7-H6, B7-H3, HM1.24, SLAMF7, Fms-like tyrosine kinase 3 (FLT-3, CD135), chondroitin sulfate proteoglycan 4 (CSPG4, melanoma-associated chondroitin sulfate proteoglycan), epidermal growth factor receptor (EGFR), Her2, Her3, IGFR, IL3R, fibroblast activating protein (FAP), CDCP1, Derlin1, Tenascin, frizzled 1-10, VEGFR2 (KDR/FLK1), VEGFR3 (FLT4, CD309), PDGFR-alpha (CD140a), PDGFR-beta (CD140b), endoglin, CLEC14, Teml-8, or Tie2. Further exemplary antigens on the tumor cell include A33, CAMPATH-1 (CDw52), Carcinoembryonic antigen (CEA), Carboanhydrase IX (MN/CA IX), de2-7, EGFRvIII, EpCAM, Ep-CAM, folate-binding protein, G250, c-Kit (CD117), CSF1R (CD115), HLA-DR, IGFR, IL-2 receptor, IL3R, MCSP (melanoma-associated cell surface chondroitin sulphate proteoglycane), Muc-1, prostate stem cell antigen (PSCA), prostate specific antigen (PSA), hK2, TAG-72 or a tumor cell neoantigen.

In some embodiments, the T cell redirecting therapeutic binds BCMA, GPRC5D, CD33, CD123, CD19, PSMA, TMEFF2, CD20, CD22, CD25, CD52, ROR1, HM1.24, CD38 or SLAMF7.

In some embodiments, the T cell redirecting therapeutic binds CD3 epsilon (CD3ε).

In some embodiments, T cell redirecting therapeutic binds CD3.

In some embodiments, the T cell redirecting therapeutic binds CD8, KI2L4, NKG2E, NKG2D, NKG2F, BTNL3, CD186, BTNL8, PD-1, CD195, or NKG2C. These antigens are more specific to CD8⁺ T cells when compared to CD3 (see e.g. Int. Pat. Publ. No. WO2018/187215).

In some embodiments, the T cell redirecting therapeutic comprises a CD3 binding domain comprising
a heavy chain complementarity determining region 1 (HCDR1) of SEQ ID NO: 33, a HCDR2 of SEQ ID NO: 34, a HCDR3 of SEQ ID NO: 35, a light chain complementarity determining region 1 (LCDR1) of SEQ ID NO: 36, a LCDR2 of SEQ ID NO: 37 and a LCDR3 of SEQ ID NO: 38;
a heavy chain variable region (VH) of SEQ ID NO: 39 and a light chain variable region (VL) of SEQ ID NO: 40;
the HCDR1 of SEQ ID NO: 74, the HCDR2 of SEQ ID NO: 75, the HCDR3 of SEQ ID NO: 76, the LCDR1 of SEQ ID NO: 77, the LCDR2 of SEQ ID NO: 78 and the LCDR3 of SEQ ID NO: 79;
the VH of SEQ ID NO: 80 and the VL of SEQ ID NO: 81;
the HCDR1, the HCDR2, the HCDR3, the LCDR1, the LCDR2 and the LCDR3 of a CD3 binding domain of SEQ ID NO: 53; or
the VH and the VL of the CD3 biding domain of SEQ ID NO: 53.

In some embodiments, the T cell redirecting therapeutic binds BCMA.

In some embodiments, the T cell redirecting therapeutic comprises a BCMA binding domain comprising the HCDR1 of SEQ ID NO: 23, the HCDR2 of SEQ ID NO: 24, the HCDR3 of SEQ ID NO: 25, the LCDR1 of SEQ ID NO: 26, the LCDR2 of SEQ ID NO: 27 and the LCDR3 of SEQ ID NO: 28, and a CD3 binding domain comprising the HCDR1 of SEQ ID NO: 33, the HCDR2 of SEQ ID NO: 34, the HCDR3 of SEQ ID NO: 35, the LCDR1 of SEQ ID NO: 36, the LCDR2 of SEQ ID NO: 37 and the LCDR3 of SEQ ID NO: 38; and/or the BCMA binding domain comprising the VH of SEQ ID NO: 29 and the VL of SEQ ID NO: 30, and the CD3 biding domain comprising the VH of SEQ ID NO: 39 and the VL of SEQ ID NO: 40.

In some embodiments, the T cell redirecting therapeutic that binds BCMA comprises a first heavy chain (HC1) of SEQ ID NO: 31, a first light chain (LC1) of SEQ ID NO: 32, a second heavy chain (HC2) of SEQ ID NO: 41, and a second light chain (LC2) of SEQ ID NO: 42.

In some embodiments, the T cell redirecting therapeutic that binds BCMA comprises ACTR cancer therapy by Seattle Genetics, AFM-26, ALLO-715, anti-BCMA allogenic CAR-T cell therapy by CRISPR Therapeutics, anti-BCMA CAR-T therapy by Sorrento Therapeutics, anti-CD19/BCMA CAR-T cell therapy by Hrain Biotechnology, BCMA CAR-T therapy by Chineo Med (Beijing), BCMA TAC-T cell therapy by Triumvira Immunologics, BCMA-CAR T cell therapy by Shanghai Unicar-Therapy Biomed, BCMA/CD3 antibody by Regeneron, CAR-NK cell therapies by NantKwest, CC-93629, CMD-505, CTX-4419, CYAD-211, HDP-101, HPN-217, P-BCMA-ALLO1, TNB-383B, bb-2121, AUTO-2, BCMA chimaeric antigen receptor therapy by Pregene, BCMA-CAR T cells by Shanghai Bioray Laboratory, BCMA-CAR-T cells by CARsgen Therapeutics, CAR-T/TCR-T cell immunotherapy by Shenzhen BinDeBio, ET-140, P-BCMA-101, REGN-5458, AMG-701, anti BCMA CAR-T cell therapy by Cellular Biomedicine Group, bb-21217, BI-836909, CC-93269, Descartes-08, IM-21, JNJ-64007957, MEDI-2228 or PF-06863135.

In some embodiments, the T cell redirecting therapeutic comprises any one of BCMA binding domains described in Int. Pat. Publ. No. WO2017/031104.

In some embodiments, the T cell redirecting therapeutic binds GPRC5D.

In some embodiments, the T cell redirecting therapeutic comprises a GPRC5D binding domain comprising the HCDR1 of SEQ ID NO: 43, the HCDR2 of SEQ ID NO: 44, the HCDR3 of SEQ ID NO: 45, the LCDR1 of SEQ ID NO: 46, the LCDR2 of SEQ ID NO: 47 and the LCDR3 of SEQ ID NO: 48, and a CD3 binding domain comprising the HCDR1 of SEQ ID NO: 33, the HCDR2 of SEQ ID NO: 34, the HCDR3 of SEQ ID NO: 35, the LCDR1 of SEQ ID NO: 36, the LCDR2 of SEQ ID NO: 37 and the LCDR3 of SEQ ID NO: 38; and/or the GPRC5D binding domain comprising the VH of SEQ ID NO: 49 and the VL of SEQ ID NO: 50, and the CD3 biding domain comprising the VH of SEQ ID NO: 39 and the VL of SEQ ID NO 40.

In some embodiments, the T cell redirecting therapeutic that binds GPRC5D comprises the HC1 of SEQ ID NO: 51, the LC1 of SEQ ID NO: 52, the HC2 of SEQ ID NO: 41, and the LC2 of SEQ ID NO: 42.

In some embodiments, the T cell redirecting therapeutic comprises GPRC5D antibodies by Eureka Therapeutics.

In some embodiments, the T cell redirecting therapeutic comprises any one of GPRC5D binding domains described in Int. Pat. Publ. No. WO2018/0037651.

In some embodiments, the T cell redirecting therapeutic binds CD33.

In some embodiments, the T cell redirecting therapeutic comprises a CD33 binding domain comprising the HCDR1 of SEQ ID NO: 84, the HCDR2 of SEQ ID NO: 85, the HCDR3 of SEQ ID NO: 86, the LCDR1 of SEQ ID NO: 87, the LCDR2 of SEQ ID NO: 88 and the LCDR3 of SEQ ID NO: 89, and a CD3 binding domain comprising the HCDR1 of SEQ ID NO: 74, the HCDR2 of SEQ ID NO: 75, the HCDR3 or SEQ ID NO: 76, the LCDR1 or SEQ ID NO: 77, the LCDR2 or SEQ ID NO: 78 and the LCDR3 of SEQ ID NO: 79; and/or the CD33 binding domain comprising the VH of SEQ ID NO: 90 and the VL of SEQ ID NO: 91, and the CD3 biding domain comprising the VH of SEQ ID NO: 80 and the VL of SEQ ID NO: 81.

In some embodiments, the T cell redirecting therapeutic that binds CD33 comprises the HC1 of SEQ ID NO: 92, the LC1 of SEQ ID NO: 93, the HC2 of SEQ ID NO: 82 and the LC2 of SEQ ID NO: 83.

In some embodiments, the T cell redirecting therapeutic that binds CD33 comprises CAR-T/TCR-T cell immunotherapy by Shenzhen BinDeBio, AMG-330, AMV-564, JNJ-67571244, ICG-144, AMG-673, CD33 CAR-T therapyINXN 3004 by, Ziopharm, huCD33-BsAb, VOR-33, HMBD-004A, GEM-333, TGB-3550 or CD33.taNK.

In some embodiments, the T cell redirecting therapeutic binds CD123.

In some embodiments, the T cell redirecting therapeutic comprises a CD123 binding domain comprising the HCDR1 of SEQ ID NO: 94, the HCDR2 of SEQ ID NO: 95, the HCDR3 of SEQ ID NO: 96, the LCDR1 of SEQ ID NO: 9, the LCDR2 of SEQ ID NO: 10, and the LCDR3 of SEQ ID NO: 59, and a CD3 binding domain comprising the HCDR1 of SEQ ID NO: 33, the HCDR2 of SEQ ID NO: 34, the HCDR3 of SEQ ID NO: 35, the LCDR1 of SEQ ID NO: 36, the LCDR2 of SEQ ID NO: 37 and the LCDR3 of SEQ ID NO: 38; and/or the CD123 binding domain comprising the VH of SEQ ID NO: 100 and the VL of SEQ ID NO: 61, and the CD3 biding domain comprising the VH of SEQ ID NO: 39 and the VL of SEQ ID NO: 40.

In some embodiments, the T cell redirecting therapeutic that binds CD123 comprises the HC1 of SEQ ID NO: 102, the LC1 of SEQ ID NO: 63, the HC2 of SEQ ID NO: 41 and the LC2 of SEQ ID NO: 42.

In some embodiments, the T cell redirecting therapeutic that binds CD123 comprises acute myeloid leukaemia therapy by TheraVectys, APVO-437, anti-CD123 CAR-T cell therapy by Nanjing Legend Biotech, APVO-436, CD123 CAR-T cell therapy by Hebei Senlang Biotechnology, flotetuzumab, IM-23, JNJ-63709178, MB-102 by Mustang Bio, UCART-123, XmAb-14045 or CD3-CD123 bispecific T-cell engager by Sanofi.

In some embodiments, the T cell redirecting therapeutic comprises any one of CD123 binding domains described in Int. Pat. Publ. No. WO2016/036937.

In some embodiments, the T cell redirecting therapeutic binds CD19.

In some embodiments, the T cell redirecting therapeutic comprises a CD19 binding domain comprising the HCDR1, the HCDR2, the HCDR3, the LCDR1, the LCDR2 and the LCDR3 of the CD19 binding domain of SEQ ID NO: 53 and a CD3 binding domain comprising the HCDR1, the HCDR2, the HCDR3, the LCDR1, the LCDR2 and the LCDR3 of the CD3 binding domain of SEQ ID NO 53; and/or the amino acid sequence of SEQ ID NO: 53.

In some embodiments, the T cell redirecting therapeutic that binds CD19 comprises axicabtagene ciloleucel, blinatumomab, tisagenlecleucel-t, AMG-562, AUTO-1 CAR-T CD19 by Cellular Biomedicine Group, CD19 chimeric antigen receptor T-cell therapy by Ziopharm, CD19-CAR-T cell therapy by ioceltech Therapeutics, CD19-CAR-T cell therapy by Marino Biotechnology, CD19-CAR-T2 cell therapy by Guangdong Zhaotai InVivo, CD19/4-1BBL armored CAR T cell therapy by Juno Therapeutics, CSG-CD19, DI-B4, ET-190, GC-007F, GC-022, human CD19 T cell therapy by HRAIN Biotechnology, humanized anti-CD19 Control CAR (3rd Gen) by Kite Pharma, ICAR-19 CAR-T cells by Immune Cell Therapy, ICTCAR-003, iPD1 CD19 eCAR T cells by Marino Biotechnology, JWCAR029, PTG-01, PZ01, Senl_1904A, Senl_1904B, UCART-19, UWC-19, AUTO-3, BinD-19, CAR-T cell therapy by Shanghai Unicar-Therapy Biomed, CAR-T/TCR-T cell immunotherapy by Shenzhen BinDeBio, CD-19 CAR-T cell therapy by Miltenyi Biotec, CD19 CAR-T cells by Shanghai Unicar-Therapy Biomed, CD19-CAR T cell therapy by Takara Bio, CD19-CART by Shanghai Bioray Laboratory, CD19-targeted chimeric antigen receptor T-cells by Sinobioway, CD19/CD20 CAR-T cell therapy by Shanghai Longyao Biotechnology, CIK-CAR.CD19, ICTCAR-011, IM-19, JCAR-014, loncastuximab tesirine, MB-CART2019.1, OXS-1550, PBCAR-0191, PCAR-019, PCAR-119, Senl-001, TI-1007, XmAb-5871, inebilizumab, lisocabtagene maraleucel, XmAb-5574, 3rd generation CD19-CART cells+mbIL15 by Eden BioCell, A-329, ALLO-501, anti-CD19 anti-CD20 Bispecific CAR redirected autologous T-cells by Beijing Doing Biomedical Co, anti-CD19 CAR NK cell therapy, by Allife Medical Science. anti-CD19/BCMA CAR-T cell therapy by Hrain Biotechnology ATA-2431, ATA-3219. AVA-008. CD19 CAR-T cell therapy by Celularity, CD19 chimeric antigen receptor T-cell therapy, 3rd generation by Ziopharm, CD19 dBiTE by Inovio, CD19 TCR-cell therapy by Bellicum, CD19-ATAC by Wilex, CD19/20 CAR-T therapy by Chineo Med (Beijing), CD19/CD22 dual targeting therapy by Eureka Therapeutics, chimeric antigen receptor T cell (CAR-T) therapies by Helix BioPharma, CMD-502, CTX-110, CYAD-04, CYAD-221, ET-019002, FT-596, FT-819, gamma-delta CAR-T therapy by TC Biopharm, ICTCAR-014, iDD-002, KITE-037, NI-2201, RB-1916, Senl_002, TACO1-CD19, TC-110, TC-310, TCB-003 or TI-7007.

In some embodiments, the T cell redirecting therapeutic binds PSMA.

In some embodiments, the T cell redirecting therapeutic comprises a PSMA binding domain comprising the HCDR1 of SEQ ID NO: 54, the HCDR2 or SEQ ID NO: 55, the HCDR3 or SEQ ID NO: 56, the LCDR1 or SEQ ID NO: 9, the LCDR2 or SEQ ID NO: 10 and the LCDR3 of SEQ ID NO: 59, and a CD3 binding domain comprising the HCDR1 of SEQ ID NO: 33, the HCDR2 of SEQ ID NO: 34, the HCDR3 of SEQ ID NO: 35, the LCDR1 of SEQ ID NO: 36, the LCDR2 of SEQ ID NO: 37 and the LCDR3 of SEQ ID NO: 38; and/or the PSMA binding domain comprising the VH of SEQ ID NO: 60 and the VL of SEQ ID NO: 61, and the CD3 biding domain comprising the VH of SEQ ID NO: 39 and the VL of SEQ ID NO: 40.

In some embodiments, the T cell redirecting therapeutic that binds PSMA comprises the HC1 of SEQ ID NO: 62, the LC1 of SEQ ID NO: 63, the HC2 of SEQ ID NO: 41 and the LC2 of SEQ ID NO: 42.

In some embodiments, the T cell redirecting therapeutic binds TMEFF2.

In some embodiments, the T cell redirecting therapeutic comprises a TMEFF2 binding domain comprising the HCDR1 of SEQ ID NO: 64, the HCDR2 of SEQ ID NO: 65, the HCDR3 of SEQ ID NO: 66, the LCDR1 of SEQ ID NO: 67, the LCDR2 of SEQ ID NO: 68 and the LCDR3 of SEQ ID NO: 69, and a CD3 binding domain comprising the HCDR1 of SEQ ID NO: 74, the HCDR2 of SEQ ID NO: 75, the HCDR3 or SEQ ID NO: 76, the LCDR1 or SEQ ID NO: 77, the LCDR2 or SEQ ID NO: 78 and the LCDR3 of SEQ ID NO: 79; and/or the TMEFF2 binding domain comprising the VH of SEQ ID NO: 70 and the VL of SEQ ID NO: 71, and the CD3 biding domain comprising the VH of SEQ ID NO: 80 and the VL of SEQ ID NO: 81.

In some embodiments, the T-cell redirecting therapeutic that binds TMEFF2 comprises the HC1 of SEQ ID NO: 72, the LC1 of SEQ ID NO: 73, the HC2 of SEQ ID NO: 82 and the LC2 of SEQ ID NO: 83.

In some embodiments, the T cell redirecting therapeutic binds CD20.

In some embodiments, the T cell redirecting therapeutic binds CD22.

In some embodiments, the T cell redirecting therapeutic binds CD25.

In some embodiments, the T cell redirecting therapeutic binds CD52.

In some embodiments, the T cell redirecting therapeutic binds ROR1.

In some embodiments, the T cell redirecting therapeutic binds HM1.24.

In some embodiments, the T cell redirecting therapeutic binds SLAMF7.

In some embodiments, the T cell redirecting therapeutic is a multispecific antibody, a chimeric antigen receptor (CAR), or a T cell comprising the CAR.

In some embodiments, the T cell redirecting therapeutic is the CAR.

In some embodiments, the T cell redirecting therapeutic is the T cell expressing the CAR.

In some embodiments, the T cell redirecting therapeutic is the multispecific antibody.

In some embodiments, the multispecific antibody is an IgG1, an IgG2, an IgG3 or an IgG4 isotype.

In some embodiments, the multispecific antibody is an IgG1 isotype.

In some embodiments, the multispecific antibody is an IgG2 isotype.

In some embodiments, the multispecific antibody is an IgG3 isotype.

In some embodiments, the multispecific antibody is an IgG4 isotype.

The multispecific antibody may be of any allotype. It is expected that allotype has no influence on properties of the multispecific antibodies, such as binding or Fc-mediated effector functions. Immunogenicity of therapeutic antibodies is associated with increased risk of infusion reactions and decreased duration of therapeutic response (Baert et al., (2003) N Engl J Med 348:602-08). The extent to which therapeutic antibodies induce an immune response in the host may be determined in part by the allotype of the antibody (Stickler et al., (2011) Genes and Immunity 12:213-21). Antibody allotype is related to amino acid sequence variations at specific locations in the constant region sequences of the antibody. Table 2 shows select IgG1, IgG2 and IgG4 allotypes.

TABLE 2

| | Amino acid residue at position of diversity (residue | | | | | | | |
|---|---|---|---|---|---|---|---|---|
| | numbering: EU Index) | | | | | | | |
| | IgG2 | | IgG4 | | IgG1 | | | |
| Allotype | 189 | 282 | 309 | 422 | 214 | 356 | 358 | 431 |
| G2m(n) | T | M | | | | | | |
| G2m(n-) | P | V | | | | | | |
| G2m(n)/(n-) | T | V | | | | | | |
| nG4m(a) | | | L | R | | | | |
| G1m(17) | | | | | K | E | M | A |
| G1m(17,1) | | | | | K | D | L | A |

In some embodiments, the multispecific antibody comprises one or more Fc substitutions that reduces binding of the multispecific antibody to a Fcγ receptor (FcγR). Substitutions that reduce binding of the multispecific antibody to the FcγR reduces the Fc effector functions such as ADCC, ADCP and/or CDC of the multispecific antibody. The specific substitutions may be made in comparison to the wild-type IgG1 of SEQ ID NO: 103 or the wild-type IgG4 of SEQ ID NO: 104.

In some embodiments, the one or more Fc substitutions is selected from the group consisting of F234A/L235A on IgG4, L234A/L235A on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4 and S228P/F234A/L235A/G236-deleted/G237A/P238S on IgG4, wherein residue numbering is according to the EU index.

In some embodiments, the one or more Fc substitutions is F234A/L235A on IgG4.

In some embodiments, the one or more Fc substitutions is L234A/L235A on IgG1.

In some embodiments, the one or more Fc substitutions is V234A/G237A/P238S/H268A/V309L/A330S/P331S on IgG2.

In some embodiments, the one or more Fc substitutions is F234A/L235A on IgG4.

In some embodiments, the one or more Fc substitutions is S228P/F234A/L235A on IgG4.

In some embodiments, the one or more Fc substitutions is N297A on all Ig isotypes.

In some embodiments, the one or more Fc substitutions is V234A/G237A on IgG2.

In some embodiments, the one or more Fc substitutions is K214T/E233P/L234V/L235A/G236-deleted/A327G/P331A/D365E/L358M on IgG1.

In some embodiments, the one or more Fc substitutions is H268Q/V309L/A330S/P331S on IgG2.

In some embodiments, the one or more Fc substitutions is S267E/L328F on IgG1. In some embodiments, the one or more Fc substitutions is L234F/L235E/D265A on IgG1.

In some embodiments, the one or more Fc substitutions is L234A/L235A/G237A/P238S/H268A/A330S/P331S on IgG1.

In some embodiments, the one or more Fc substitutions is S228P/F234A/L235A/G237A/P238S on IgG4 and S228P/F234A/L235A/G236-deleted/G237A/P238S on IgG4.

In some embodiments, the multispecific antibody further comprises a S228P substitution.

In some embodiments, the multispecific antibody comprises one or more asymmetric substitutions in a first CH3 domain or in a second CH3 domain, or in both the first CH3 domain and the second CH3 domain.

In some embodiments, the one or more asymmetric substitutions is selected from the group consisting of F450L/K409R, wild-type/F409L_R409K, T366Y/F405A, T366W/F405W, F405W/Y407A, T394W/Y407T, T394S/Y407A, T366W/T394S, F405W/T394S and T366W/T366S_L368A_Y407V, L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F and T350V_L351Y_F405A_Y407V/T350V_T366L_K392L_T394W.

In some embodiments, the one or more asymmetric substitutions is F450L/K409R.

In some embodiments, the one or more asymmetric substitutions is wild-type/F409L_R409K.

In some embodiments, the one or more asymmetric substitutions is T366Y/F405A.

In some embodiments, the one or more asymmetric substitutions is T366W/F405W.

In some embodiments, the one or more asymmetric substitutions is F405W/Y407A.

In some embodiments, the one or more asymmetric substitutions is T394W/Y407T.

In some embodiments, the one or more asymmetric substitutions is T394S/Y407A.

In some embodiments, the one or more asymmetric substitutions is T366W/T394S.

In some embodiments, the one or more asymmetric substitutions is F405W/T394S.

In some embodiments, the one or more asymmetric substitutions is T366W/T366S_L368A_Y407V.

In some embodiments, the one or more asymmetric substitutions is L351Y_F405A_Y407V/T394W.

In some embodiments, the one or more asymmetric substitutions is T366I_K392M_T394W/F405A_Y407V.

In some embodiments, the one or more asymmetric substitutions is T366L_K392M_T394W/F405A_Y407V.

In some embodiments, the one or more asymmetric substitutions is L351Y_Y407A/T366A_K409F.

In some embodiments, the one or more asymmetric substitutions is L351Y_Y407A/T366V_K409F.

In some embodiments, the one or more asymmetric substitutions is Y407A/T366A_K409F.

In some embodiments, the one or more asymmetric substitutions is T350V_L351Y_F405A_Y407V/T350V_T366L_K392L_T394W.

In some embodiments, the cancer is a hematological malignancy or a solid tumor.

In some embodiments, the hematological malignancy is a multiple myeloma, a smoldering multiple myeloma, a monoclonal gammopathy of undetermined significance (MGUS), an acute lymphoblastic leukemia (ALL), a diffuse large B-cell lymphoma (DLBCL), a Burkitt's lymphoma (BL), a follicular lymphoma (FL), a mantle-cell lymphoma (MCL), Waldenstrom's macroglobulinema, a plasma cell leukemia, a light chain amyloidosis (AL), a precursor B-cell lymphoblastic leukemia, a precursor B-cell lymphoblastic leukemia, an acute myeloid leukemia (AML), a myelodysplastic syndrome (MDS), a chronic lymphocytic leukemia (CLL), a B cell malignancy, a chronic myeloid leukemia (CML), a hairy cell leukemia (HCL), a blastic plasmacytoid dendritic cell neoplasm, Hodgkin's lymphoma, non-Hodgkin's lymphoma, a marginal zone B-cell lymphoma (MZL), a mucosa-associated lymphatic tissue lymphoma (MALT), plasma cell leukemia, anaplastic large-cell lymphoma (ALCL), leukemia or lymphoma.

In some embodiments, the hematological malignancy is the multiple myeloma.

In some embodiments, the multiple myeloma is a newly diagnosed multiple myeloma.

In some embodiments, the multiple myeloma is a relapsed or a refractory multiple myeloma.

In some embodiments, the multiple myeloma is a high-risk multiple myeloma. Subjects with high-risk multiple myeloma are known to relapse early and have poor prognosis and outcome. Subjects can be classified as having high-risk multiple myeloma is they have one or more of the following cytogenetic abnormalities: t(4; 14)(p16; q32), t(14; 16)(q32; q23), del17p, 1qAmp, t(4; 14)(p16; q32) and t(14; 16)(q32; q23), t(4; 14)(p16; q32) and del17p, t(14; 16)(q32; q23) and del17p, or t(4; 14)(p16; q32), t(14; 16)(q32; q23) and del17p.

In some embodiments, the subject having the high-risk multiple myeloma has one or more chromosomal abnormalities comprising: t(4; 14)(p16; q32), t(14; 16)(q32; q23), del17p, 1qAmp, t(4; 14)(p16; q32) and t(14; 16)(q32; q23), t(4; 14)(p16; q32) and del17p, t(14; 16)(q32; q23) and del17p; or t(4; 14)(p16; q32), t(14; 16)(q32; q23) and del17p, or any combination thereof.

Various qualitative and/or quantitative methods may be used to determine relapse or refractory nature of the disease. Symptoms that may be associated are for example a decline or plateau of the well-being of the patient or re-establishment or worsening of various symptoms associated with solid tumors, and/or the spread of cancerous cells in the body from one location to other organs, tissues or cells.

The cytogenetic abnormalities can be detected for example by fluorescent in situ hybridization (FISH). In chromosomal translocations, an oncogene is translocated to the IgH region on chromosome 14q32, resulting in dysregulation of these genes. t(4; 14)(p16; q32) involves translocation of fibroblast growth factor receptor 3 (FGFR3) and multiple myeloma SET domain containing protein (MMSET) (also called WHSC1/NSD2), and t(14; 16)(q32; q23) involves translocation of the MAF transcription factor C-MAF. Deletion of 17p (del17p) involves loss of the p53 gene locus.

In some embodiments, the multiple myeloma is relapsed or refractory to treatment with the anti-CD38 antibody, lenalinomide, bortezomib, pomalidomide, carfilzomib, elotozumab, ixazomib, melphalan or thalidomide, or any combination thereof.

In some embodiments, the multiple myeloma is relapsed or refractory to treatment with the anti-CD38 antibody. In some embodiments, the multiple myeloma is relapsed or refractory to treatment with lenalinomide. In some embodiments, the multiple myeloma is relapsed or refractory to treatment with bortezomib. In some embodiments, the multiple myeloma is relapsed or refractory to treatment with pomalidomide. In some embodiments, the multiple myeloma is relapsed or refractory to treatment with carfilzomib. In some embodiments, the multiple myeloma is relapsed or refractory to treatment with elotozumab. In some embodiments, the multiple myeloma is relapsed or refractory to treatment with ixazomib. In some embodiments, the multiple myeloma is relapsed or refractory to treatment with melphalan. In some embodiments, the multiple myeloma is relapsed or refractory to treatment with or thalidomide.

In some embodiments, the hematological malignancy is the AML.

In some embodiments, the AML is AML with at least one genetic abnormality, AML with multilineage dysplasia, therapy-related AML, undifferentiated AML, AML with minimal maturation, AML with maturation, acute myelomonocytic leukemia, acute monocytic leukemia, acute erythroid leukemia, acute megakaryoblastic leukemia, acute basophilic leukemia, acute panmyelosis with fibrosis or myeloid sarcoma.

In some embodiments, the AML is AML with at least one genetic abnormality. In some embodiments, the AML is AML with multilineage dysplasia. In some embodiments, the AML is therapy-related AML. In some embodiments, the AML is undifferentiated AML. In some embodiments, the AML is AML with minimal maturation. In some embodiments, the AML is AML with maturation. In some embodiments, the AML is acute myelomonocytic leukemia. In some embodiments, the AML is acute monocytic leukemia. In some embodiments, the AML is acute erythroid leukemia. In some embodiments, the AML is acute megakaryoblastic leukemia. In some embodiments, the AML is acute basophilic leukemia. In some embodiments, the AML is acute panmyelosis with fibrosis. In some embodiments, the AML is myeloid sarcoma.

In some embodiments, the at least one genetic abnormality is a translocation between chromosomes 8 and 21, a translocation or an inversion in chromosome 16, a translocation between chromosomes 15 and 17, changes in chromosome 11, or mutation in fms-related tyrosine kinase 3 (FLT3), nucleophosmin (NPM1), isocitrate dehydrogenase 1 (IDH1), isocitrate dehydrogenase 2 (IDH2), DNA (cytosine-5)-methyltransferase 3 (DNMT3A), CCAAT/enhancer binding protein alpha (CEBPA), U2 small nuclear RNA auxiliary factor 1 (U2AF1), enhancer of zeste 2 polycomb repressive complex 2 subunit (EZH2), structural maintenance of chromosomes 1A (SMC1A) or structural maintenance of chromosomes 3 (SMC3).

In some embodiments, the at least one genetic abnormality is the translocation between chromosomes 8 and 21. In some embodiments, the at least one genetic abnormality is the translocation or an inversion in chromosome 16. In some embodiments, the at least one genetic abnormality is the translocation between chromosomes 15 and 17. In some embodiments, the at least one genetic abnormality is changes in chromosome 11. In some embodiments, the at least one genetic abnormality is the mutation in fms-related tyrosine kinase 3 (FLT3). In some embodiments, the at least one genetic abnormality is the mutation in nucleophosmin (NPM1). In some embodiments, the at least one genetic abnormality is the mutation in isocitrate dehydrogenase 1 (IDH1). In some embodiments, the at least one genetic abnormality is the mutation in isocitrate dehydrogenase 2 (IDH2). In some embodiments, the at least one genetic abnormality is the mutation in DNA (cytosine-5)-methyltransferase 3 (DNMT3A). In some embodiments, the at least one genetic abnormality is the mutation in CCAAT/enhancer binding protein alpha (CEBPA). In some embodiments, the at least one genetic abnormality is the mutation in U2 small nuclear RNA auxiliary factor 1(U2AF1). In some embodiments, the at least one genetic abnormality is the mutation in enhancer of zeste 2 polycomb repressive complex 2 subunit (EZH2). In some embodiments, the at least one genetic abnormality is the mutation in structural maintenance of chromosomes 1A (SMC1A). In some embodiments, the at least one genetic abnormality is the mutation in structural maintenance of chromosomes 3 (SMC3).

In some embodiments, the at least one genetic abnormality is a translocation t(8; 21)(q22; q22), an inversion inv(16)(p13; q22), a translocation t(16; 16)(p13; q22), a translocation t(15; 17)(q22; q12), a mutation FLT3-ITD, mutations R132H or R100Q/R104V/F108L/R119Q/1130V in IDH1 or mutations R140Q or R172 in IDH2.

In some embodiments, the at least one genetic abnormality is the translocation t(8; 21)(q22; q22). In some embodiments, the at least one genetic abnormality is the inversion inv(16)(p13; q22). In some embodiments, the at least one genetic abnormality is the translocation t(16; 16)(p13; q22). In some embodiments, the at least one genetic abnormality is the translocation t(15; 17)(q22; q12). In some embodiments, the at least one genetic abnormality is the mutation FLT3-ITD. In some embodiments, the at least one genetic abnormality is the mutation R132H in IDH1. In some embodiments, the at least one genetic abnormality is the mutation R100Q/R104V/F108L/R119Q/1130V in IDH1. In some embodiments, the at least one genetic abnormality is the mutation R140Q in IDH2. In some embodiments, the at least one genetic abnormality is the mutation R172 in IDH2.

In some embodiments, the hematological malignancy is the ALL.

In some embodiments, the ALL is B-cell lineage ALL, T-cell lineage ALL, adult ALL or pediatric ALL.

In some embodiments, the ALL is B-cell lineage ALL. In some embodiments, the ALL is T-cell lineage ALL. In some embodiments, the ALL is adult ALL. In some embodiments, the ALL is pediatric ALL.

In some embodiments, the subject with ALL has a Philadelphia chromosome or is resistant or has acquired resistance to treatment with a BCR-ABL kinase inhibitor.

In some embodiments, the subject with ALL has the Philadelphia chromosome. In some embodiments, the subject with ALL is resistant or has acquired resistance to treatment with a BCR-ABL kinase inhibitor.

The Ph chromosome is present in about 20% of adults with ALL and a small percentage of children with ALL and is associated with poor prognosis. At a time of relapse, patients with Ph+ positive ALL may be on tyrosine kinase inhibitor (TKI) regimen and may have therefore become resistant to the TKI. The anti-CD38 antibodies may thus be administered to a subject who has become resistant to selective or partially selective BCR-ABL inhibitors. Exemplary BCR-ABL inhibitors are for example imatinib, dasatinib, nilotinib, bosutinib, ponatinib, bafetinib, saracatinib, tozasertib or danusertib.

Other chromosomal rearrangements identified in B-lineage ALL patients are t(v; 11q23) (MLL rearranged), t(1; 19)(q23; p13.3); TCF3-PBX1 (E2A-PBX1), t(12; 21)(p13; q22); ETV6-RUNX1 (TEL-AML1) and t(5; 14)(q31; q32); IL3-IGH.

In some embodiments, the subject has ALL with t(v; 11q23) (MLL rearranged), t(1; 19)(q23; p13.3); TCF3-PBX1 (E2A-PBX1), t(12; 21)(p13; q22); ETV6-RUNX1 (TEL-AML1) or t(5; 14)(q31; q32); IL3-IGH chromosomal rearrangement.

Chromosomal rearrangements can be identified using well known methods, for example fluorescent in situ hybridization, karyotyping, pulsed field gel electrophoresis, or sequencing.

In some embodiments, the hematological malignancy is the smoldering multiple myeloma.

In some embodiments, the hematological malignancy is the MGUS.

In some embodiments, the hematological malignancy is the ALL.

In some embodiments, the hematological malignancy is the DLBLC.

In some embodiments, the hematological malignancy is the BL.

In some embodiments, the hematological malignancy is the FL.

In some embodiments, the hematological malignancy is the MCL.

In some embodiments, the hematological malignancy is Waldenstrom's macroglobulinema.

In some embodiments, the hematological malignancy is the plasma cell leukemia.

In some embodiments, the hematological malignancy is the AL.

In some embodiments, the hematological malignancy is the precursor B-cell lymphoblastic leukemia.

In some embodiments, the hematological malignancy is the precursor B-cell lymphoblastic leukemia.

In some embodiments, the hematological malignancy is the myelodysplastic syndrome (MDS).

In some embodiments, the hematological malignancy is the CLL.

In some embodiments, the hematological malignancy is the B cell malignancy.

In some embodiments, the hematological malignancy is the CML.

In some embodiments, the hematological malignancy is the HCL.

In some embodiments, the hematological malignancy is the blastic plasmacytoid dendritic cell neoplasm.

In some embodiments, the hematological malignancy is Hodgkin's lymphoma.

In some embodiments, the hematological malignancy is non-Hodgkin's lymphoma.

In some embodiments, the hematological malignancy is the MZL.

In some embodiments, the hematological malignancy is the MALT.

In some embodiments, the hematological malignancy is the plasma cell leukemia.

In some embodiments, the hematological malignancy is the ALCL.

In some embodiments, the hematological malignancy is leukemia.

In some embodiments, the hematological malignancy is lymphoma.

In some embodiments, the solid tumor is a prostate cancer, a lung cancer, a non-small cell lung cancer (NSCLC), a liver cancer, a cervical cancer, a colon cancer, a breast cancer, an ovarian cancer, an endometrial cancer, a pancreatic cancer, a melanoma, an esophageal cancer, a gastric cancer, a stomach cancer, a renal carcinoma, a bladder cancer, a hepatocellular carcinoma, a renal cell carcinoma, an urothelial carcinoma, a head and neck cancer, a glioma, a glioblastoma, a colorectal cancer, a thyroid cancer, epithelial cancers, adenocarcinomas or advanced solid tumors.

In some embodiments, the solid tumor is the prostate cancer.

In some embodiments, the solid tumor is the lung cancer.

In some embodiments, the solid tumor is the non-small cell lung cancer (NSCLC).

In some embodiments, the solid tumor is the liver cancer.

In some embodiments, the solid tumor is the cervical cancer.

In some embodiments, the solid tumor is the colon cancer.

In some embodiments, the solid tumor is the breast cancer.

In some embodiments, the solid tumor is the ovarian cancer.

In some embodiments, the solid tumor is the endometrial cancer.

In some embodiments, the solid tumor is the pancreatic cancer.

In some embodiments, the solid tumor is the melanoma.

In some embodiments, the solid tumor is the esophageal cancer.

In some embodiments, the solid tumor is the gastric cancer.

In some embodiments, the solid tumor is the stomach cancer.

In some embodiments, the solid tumor is the renal carcinoma.

In some embodiments, the solid tumor is the bladder cancer.

In some embodiments, the solid tumor is the hepatocellular carcinoma.

In some embodiments, the solid tumor is the renal cell carcinoma.

In some embodiments, the solid tumor is the urothelial carcinoma.

In some embodiments, the solid tumor is the head and neck cancer.

In some embodiments, the solid tumor is the glioma.

In some embodiments, the solid tumor is the glioblastoma.

In some embodiments, the solid tumor is the colorectal cancer.

In some embodiments, the solid tumor is the thyroid cancer.

In some embodiments, the solid tumor is epithelial cancers.

In some embodiments, the solid tumor is adenocarcinomas.

In some embodiments, the solid tumor is advanced solid tumors.

In some embodiments, the prostate cancer is a relapsed, a refractory, a malignant or a castration resistant prostate cancer, or any combination thereof.

In some embodiments, the prostate cancer is a relapsed prostate cancer. In some embodiments, the prostate cancer is a refractory prostate cancer. In some embodiments, the prostate cancer is a malignant prostate cancer. In some embodiments, the prostate cancer is a castration resistant prostate cancer.

In some embodiments, the anti-CD38 antibody comprises the HCDR1 of SEQ ID NO: 6, the HCDR2 of SEQ ID NO: 7, the HCDR3 of SEQ ID NO: 8, the LCDR1 of SEQ ID NO: 9, the LCDR2 of SEQ ID NO: 10 and the LCDR3 of SEQ ID NO: 11.

In some embodiments, the anti-CD38 antibody comprises the VH of SEQ ID NO: 4 and the VL of SEQ ID NO: 5.

In some embodiments, the anti-CD38 antibody is an IgG1 isotype.

In some embodiments, the anti-CD38 antibody comprises the HC of SEQ ID NO: 12 and the LC of SEQ ID NO: 13.

Other anti-CD38 antibodies used in the methods of the invention may be known antibodies, such as mAb003 comprising the VH and the VL sequences of SEQ ID NOs: 14 and 15, respectively and described in U.S. Pat. No. 7,829,673. The VH and the VL of mAb003 may be expressed as IgG1/κ; mAb024 comprising the VH and the VL sequences of SEQ ID NOs: 16 and 17, respectively, described in U.S. Pat. No. 7,829,673. The VH and the VL of mAb024 may be expressed as IgG1/κ; MOR-202 (MOR-03087) comprising the VH and the VL sequences of SEQ ID NOs: 18 and 19, respectively, described in U.S. Pat. No. 8,088,896. The VH and the VL of MOR-202 may be expressed as IgG1/κ; or isatuximab; comprising the VH and the VL sequences of SEQ ID NOs: 20 and 21, respectively, described in U.S. Pat. No. 8,153,765. The VH and the VL of Isatuximab may be expressed as IgG1/κ.

(Daratumumab VH)
SEQ ID NO: 4
EVQLLESGGGLVQPGGSLRLSCAVSGFTFNSFAMSWVRQAPGKGLEWVSA
ISGSGGGTYYADSVKGRFTISRDNSKNTLYLQMNSLRAEDTAVYFCAKDK
ILWFGEPVFDYWGQGTLVTVSS
(Daratumumab VL)
SEQ ID NO: 5
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPPTFGQ
GTKVEIK
(Daratumumab HCDR1)
SEQ ID NO: 6
SFAMS
(Daratumumab HCDR2)
SEQ ID NO: 7
AISGSGGGTYYADSVKG
(Daratumumab HCDR3)
SEQ ID NO: 8
DKILWFGEPVFDY
(Daratumumab LCDR1)
SEQ ID NO: 9
RASQSVSSYLA
(Daratumumab LCDR2)
SEQ ID NO: 10
DASNRAT
(Daratumumab LCDR3)
SEQ ID NO: 11
QQRSNWPPTF
(Daratumumab HC)
SEQ ID NO: 12
EVQLLESGGGLVQPGGSLRLSCAVSGFTFNSFAMSWVRQAPGKGLEWVSA
ISGSGGGTYYADSVKGRFTISRDNSKNTLYLQMNSLRAEDTAVYFCAKDK
ILWFGEPVFDYWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK
(Daratumumab LC)
SEQ ID NO: 13
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
SEQ ID NO: 14
QVQLVQSGAEVKKPGSSVKVSCKASGGTFSSYAFSWVRQAPGQGLEWMGR
VIPFLGIANSAQKFQGRVTITADKSTSTAYMDLSSLRSEDTAVYYCARDD
IAALGPFDYWGQGTLVTVSSAS
SEQ ID NO: 15
DIQMTQSPSSLSASVGDRVTITCRASQGISSWLAWYQQKPEKAPKSLIYA
ASSLQSGVPSRFSGSGSGTDFTLTISSLQPEDFATYYCQQYNSYPRTFGQ
GTKVEIK
SEQ ID NO: 16
EVQLVQSGAEVKKPGESLKISCKGSGYSFSNYWIGWVRQMPGKGLEWMGI
IYPHDSDARYSPSFQGQVTFSADKSISTAYLQWSSLKASDTAMYYCARHV
GWGSRYWYFDLWGRGTLVTVSS
SEQ ID NO: 17
EIVLTQSPATLSLSPGERATLSCRASQSVSSYLAWYQQKPGQAPGLLIYD
ASNRASGIPARFSGSGSGTDFTLTISSLEPEDFAVYYCQQRSNWPLTFGG
GTKVEIK
SEQ ID NO: 18
QVQLVESGGGLVQPGGSLRLSCAASGFTFSSYYMNWVRQAPGKGLEWVSG
ISGDPSNTYYADSVKGRFTISRDNSKNTLYLQMNSLRAEDTAVYYCARDL
PLVYTGFAYWGQGTLVTVSS
SEQ ID NO: 19
DIELTQPPSVSVAPGQTARISCSGDNLRHYYVYWYQQKPGQAPVLVIYGD
SKRPSGIPERFSGSNSGNTATLTISGTQAEDEADYYCQTYTGGASLVFGG
GTKLTVLGQ
SEQ ID NO 20:
QVQLVQSGAEVAKPGTSVKLSCKASGYTFTDYWMQWVKQRPGQGLEWIGT
IYPGDGDTGYAQKFQGKATLTADKSSKTVYMHLSSLASEDSAVYYCARGD
YYGSNSLDYWGQGTSVTVSS
SEQ ID NO: 21:
DIVMTQSHLSMSTSLGDPVSITCKASQDVSTVVAWYQQKPGQSPRRLIYS
ASYRYIGVPDRFTGSGAGTDFTFTISSVQAEDLAVYYCQQHYSPPYTFGG
GTKLEIK

In some embodiments, the anti-CD38 antibody comprises
the VH of SEQ ID NO: 14 and the VL of SEQ ID NO: 15;
the VH of SEQ ID NO: 16 and the VL of SEQ ID NO: 17;
the VH of SEQ ID NO: 18 and the VL of SEQ ID NO: 19; or
the VH of SEQ ID NO: 20 and the VL of SEQ ID NO: 21.

In some embodiments, the anti-CD38 antibody is an IgG1 isotype.

In some embodiments, the T-cell redirecting therapeutic is a BCMAxCD3 bispecific antibody, a GPRC5DxCD3 bispecific antibody, a CD33xCD3 bispecific antibody, a CD19xCD3 bispecific antibody, a CD123xCD3 bispecific antibody, a PSMAxCD3 bispecific antibody, or a TMEFF2xCD3 bispecific antibody.

In some embodiments, the T-cell redirecting therapeutic is the BCMAxCD3 bispecific antibody.

In some embodiments, the T-cell redirecting therapeutic is the GPRC5DxCD3 bispecific antibody.

In some embodiments, the T-cell redirecting therapeutic is the CD33xCD3 bispecific antibody, In some embodiments, the T-cell redirecting therapeutic is the CD19xCD3 bispecific antibody.

In some embodiments, the T-cell redirecting therapeutic is the CD123xCD3 bispecific antibody.

In some embodiments, the T-cell redirecting therapeutic is the PSMAxCD3 bispecific antibody.

In some embodiments, the T-cell redirecting therapeutic is the TMEFF2xCD3 bispecific antibody.

In some embodiments, the method further comprises administering to the subject one or more anti-cancer therapies.

In some embodiments, the one or more anti-cancer therapies is selected from the group consisting of an autologous stem cell transplant (ASCT), radiation, surgery, a chemotherapeutic agent, an immunomodulatory agent and a targeted cancer therapy.

In some embodiments, the one or more anti-cancer therapies is the autologous stem cell transplant (ASCT). In some embodiments, the one or more anti-cancer therapies is radiation. In some embodiments, the one or more anti-cancer therapies is surgery. In some embodiments, the one or more anti-cancer therapies is the chemotherapeutic agent. In some embodiments, the one or more anti-cancer therapies is the immunomodulatory agent. In some embodiments, the one or more anti-cancer therapies is targeted cancer therapy.

In some embodiments, the one or more anti-cancer therapies is selected from the group consisting of lenalidomide, thalidomide, pomalidomide, bortezomib, carfilzomib, elotozumab, ixazomib, melphalan, dexamethasone, vincristine, cyclophosphamide, hydroxydaunorubicin, prednisone, rituximab, imatinib, dasatinib, nilotinib, bosutinib, ponatinib, bafetinib, saracatinib, tozasertib or danusertib, cytarabine, daunorubicin, idarubicin, mitoxantrone, hydroxyurea, decitabine, cladribine, fludarabine, topotecan, etoposide 6-thioguanine, corticosteroid, methotrexate, 6-mercaptopurine, azacitidine, arsenic trioxide and all-trans retinoic acid, or any combination thereof.

In some embodiments, the anti-CD38 antibody is administered at a dose of between about 8 mg/kg and about 16 mg/kg.

In some embodiments, the anti-CD38 antibody is administered or provided for administration in a pharmaceutical composition comprising between about 20 mg/mL to about 120 mg/mL of the anti-CD38 antibody in about 25 mM acetic acid, about 60 mM sodium chloride, about 140 mannitol and about 0.04% w/v polysorbate-20 (PS-20); at pH about 5.5.

In some embodiments, the anti-CD38 antibody is administered or provided for administration in a pharmaceutical composition comprising about 1,800 mg of the anti-CD38 antibody and about 30,000 U of rHuPH20.

In some embodiments, the anti-CD38 antibody is administered or provided for administration in a pharmaceutical composition comprising about 120 mg/mL of the anti-CD38 antibody and about 2,000 U/mL of rHuPH20.

In some embodiments, the -CD38 antibody is administered or provided for administration in a pharmaceutical composition comprising
between about 5 mM and about 15 mM histidine;
between about 100 mM and about 300 mM sorbitol;
between about 0.01% w/v and about 0.04% w/v PS-20; and
between about 1 mg/mL and about 2 mg/mL methionine, at a pH of about 5.5-5.6.

In some embodiments, the anti-CD38 antibody is administered or provided for administration in a pharmaceutical composition comprising
about 1,800 mg of the anti-CD38 antibody;
about 30,000 U of rHuPH20;
about 10 mM histidine;
about 300 mM sorbitol;
about 0.04% (w/v) PS-20; and
about 1 mg/mL methionine, at a pH of about 5.6.

In some embodiments, the anti-CD38 antibody is administered or provided for administration in a pharmaceutical composition comprising
about 120 mg/mL of the anti-CD38 antibody;
about 2,000 U/mL of rHuPH20;
about 10 mM histidine;
about 300 mM sorbitol;
about 0.04% (w/v) PS-20; and
about 1 mg/mL methionine, at a pH of about 5.6.

### Combinations of Anti-CD38 Antibodies and BCMAxCD3 Bispecific Antibodies

The disclosure also provides a method of treating a cancer in a subject, comprising administering a therapeutically effective amount of a BCMAxCD3 bispecific antibody and an anti-CD38 antibody to the subject to treat the cancer.

The disclosure also provides a method of treating a cancer in a subject, comprising administering a therapeutically effective amount of a BCMAxCD3 bispecific antibody to the subject to treat the cancer, wherein the subject has been treated with an anti-CD38 antibody prior to administering the BCMAxCD3 bispecific antibody.

The disclosure also provides a method of treating a cancer in a subject, comprising administering a therapeutically effective amount of a BCMAxCD3 bispecific antibody to the subject to treat the cancer, wherein the subject is relapsed or refractory to treatment with a prior anti-cancer therapeutic.

T cell redirecting therapeutics such as BCMAxCD3 bispecific antibodies such as JNJ-957 redirect T cells to the BCMA-positive tumor cells such as multiple myeloma cells, which is followed by perforin/granzyme release or activation of the FASL/FAS pathway, and ultimately death of the BCMA-positive tumor cell death. Efficacy of the T cell redirecting therapeutics such as BCMAxCD3 bispecific antibodies may hence be influenced by the availability and activity of the recruited T cells as well as possible modulated expression of a tumor associated antigen such as BCMA on tumor cells.

In some embodiments, the cancer is a BCMA expressing cancer.

B-cell maturation antigen (BCMA) is a cell membrane bound tumor necrosis factor receptor family member involved in differentiation of B-cells to plasma cells. Expression of BCMA is restricted to the B-cell lineage where it is predominantly expressed in the interfollicular region of germinal centers and on differentiated plasma cells and plasmablasts. BCMA is virtually absent on naïve and memory B cells (Tai and Anderson, Immunotherapy 7: 1187-99, 2015).

In some embodiments, the cancer is a hematologic malignancy.

In some embodiments, the cancer is a multiple myeloma, a smoldering myeloma, a monoclonal gammopathy of undetermined significance (MGUS), a B-cell acute lymphoblastic leukemia, a diffuse large B-cell lymphoma, a Burkitt's lymphoma, a follicular lymphoma, a mantle-cell lymphoma, Waldenstrom's macroglobulinema, plasma cell leukemia, light chain amyloidosis or non-Hodgkin's lymphoma. An experienced physician makes the cancer diagnosis.

In some embodiments, the subject is relapsed or refractory to treatment with the anti-CD38 antibody or lenalinomide, or a combination thereof.

In some embodiments, the subject is relapsed or refractory to treatment with the anti-CD38 antibody. In some embodiments, the subject is relapsed or refractory to treatment with lenalinomide.

In some embodiments, the subject is relapsed or refractory to treatment with a prior anti-cancer therapeutic, such as a therapeutic used to treat multiple myeloma or other hematological malignancies.

In some embodiments, the subject is refractory or relapsed to treatment with THALOMID® (thalidomide), REVLIMID® (lenalidomide), POMALYST® (pomalidomide), VELCADE® (bortezomib), NINLARO (ixazomib), KYPROLIS® (carfilzomib), FARADYK® (panobinostat), AREDIA® (pamidronate), ZOMETA® (zoledronic acid), DARZALEX® (daratumumab), elotozumab or melphalan.

In some embodiments, the subject is relapsed to treatment with DARZALEX® (daratumumab).

In some embodiments, the BCMAxCD3 bispecific antibody and the anti-CD38 antibody are antigen binding fragments. Exemplary antigen binding fragments are Fab, F(ab′)2, Fd and Fv fragments.

In some embodiments, the BCMAxCD3 bispecific antibody is chimeric, humanized or human.

In some embodiments, the BCMAxCD3 bispecific antibody is an IgG1, an IgG2, an IgG3 or an IgG4 isotype.

In some embodiments, the BCMAxCD3 bispecific antibody is an IgG4 isotype.

In some embodiments, the BCMAxCD3 bispecific antibody comprises a BCMA binding domain comprising the HCDR1 of SEQ ID NO: 23, the HCDR2 of SEQ ID NO: 24, the HCDR3 of SEQ ID NO: 25, the LCDR1 of SEQ ID NO: 26, the LCDR2 of SEQ ID NO: 27 and the LCDR3 of SEQ ID NO: 28 and a CD3 binding domain comprising the HCDR1 of SEQ ID NO: 33, the HCDR2 of SEQ ID NO: 34, the HCDR3 of SEQ ID NO: 35, the LCDR1 of SEQ ID NO: 36, the LCDR2 of SEQ ID NO: 37 and the LCDR3 of SEQ ID NO: 38.

In some embodiments, the BCMA binding domain comprises the VH of SEQ ID NO: 29 and the VL of SEQ ID NO: 30 and the CD3 biding domain comprises the VH of SEQ ID NO: 39 and the VL of SEQ ID NO: 40.

In some embodiments, the BCMAxCD3 bispecific antibody is an IgG4 isotype and comprises phenylalanine at position 405 and arginine at position 409 in a first heavy chain (HC1) and leucine at position 405 and lysine at position 409 in a second heavy chain (HC2), wherein residue numbering is according to the EU Index.

In some embodiments, the BCMAxCD3 bispecific antibody further comprises proline at position 228, alanine at position 234 and alanine at position 235 in both the HC1 and the HC2.

In some embodiments, the BCMAxCD3 bispecific antibody comprises the HC1 of SEQ ID NO: 31, a first light chain (LC1) of SEQ ID NO: 32, the HC2 of SEQ ID NO: 41 and a second light chain (LC2) of SEQ ID NO: 42.

In some embodiments, the BCMAxCD3 bispecific antibody is BI 836909, PF-06863135, AMG-701 or CC-93269.

In some embodiments, the anti-CD38 antibody comprises the HCDR1 of SEQ ID NO: 6, the HCDR2 of SEQ ID NO: 7, the HCDR3 of SEQ ID NO: 8, the LCDR1 of SEQ ID NO: 9, the LCDR2 of SEQ ID NO: 10 and the LCDR3 of SEQ ID NO: 11.

In some embodiments, the anti-CD38 antibody comprises the VH of SEQ ID NO: 4 and the VL of SEQ ID NO: 5.

In some embodiments, the anti-CD38 antibody comprises a heavy chain (HC) of SEQ ID NO: 12 and a light chain (LC) of SEQ ID NO: 13.

In some embodiments, the anti-CD38 antibody is DARZALEX® (daratumumab).

In some embodiments, the anti-CD38 antibody comprises
the VH of SEQ ID NO: 14 and the VL of SEQ ID NO: 15;
the VH of SEQ ID NO: 16 and the VL of SEQ ID NO: 17;
the VH of SEQ ID NO: 18 and the VL of SEQ ID NO: 19; or
the VH of SEQ ID NO: 20 and the VL of SEQ ID NO: 21.

In some embodiments, the anti-CD38 antibody is chimeric, humanized or human.

In some embodiments, the anti-CD38 antibody is an IgG1, an IgG2, an IgG3 or an IgG4 isotype.

In some embodiments, the anti-CD38 antibody is an IgG1 isotype.

In some embodiments, the anti-CD38 antibody is administered at a dose of between about 8 mg/kg and about 16 mg/kg.

In some embodiments, the BCMAxCD3 bispecific antibody and the anti-CD38 antibody are administered by an intravenous injection.

In some embodiments, the BCMAxCD3 bispecific antibody is administered by an intravenous injection and the anti-CD38 antibody is administered by a subcutaneous injection.

In some embodiments, the BCMAxCD3 bispecific antibody and the anti-CD38 antibody is administered by a subcutaneous injection.

In some embodiments, the method further comprises administering to the subject one or more anti-cancer therapies.

In some embodiments, the one or more anti-cancer therapies is selected from the group consisting of an autologous stem cell transplant (ASCT), radiation, surgery, a chemotherapeutic agent, an immunomodulatory agent and a targeted cancer therapy.

In some embodiments, the one or more anti-cancer therapies is selected from the group consisting of lenalidomide, thalidomide, pomalidomide, bortezomib, carfilzomib, elotozumab, ixazomib, melphalan, prednisone or dexamethasone, or any combination thereof.

In some embodiments, the anti-CD38 antibody is administered or provided for administration in a pharmaceutical composition comprising between about 20 mg/mL to about 120 mg/mL of the anti-CD38 antibody in about 25 mM acetic acid, about 60 mM sodium chloride, about 140 mannitol and about 0.04% w/v polysorbate-20 (PS-20); at pH about 5.5.

In some embodiments, the anti-CD38 antibody is administered or provided for administration in a pharmaceutical composition comprising about 1,800 mg of the anti-CD38 antibody and about 30,000 U of rHuPH20.

In some embodiments, the anti-CD38 antibody is administered or provided for administration in a pharmaceutical composition comprising about 120 mg/mL of the anti-CD38 antibody and about 2,000 U/mL of rHuPH20.

In some embodiments, the anti-CD38 antibody is administered or provided for administration in a pharmaceutical composition comprising
between about 5 mM and about 15 mM histidine;
between about 100 mM and about 300 mM sorbitol;
between about 0.01% w/v and about 0.04% w/v PS-20; and
between about 1 mg/mL and about 2 mg/mL methionine, at a pH of about 5.5-5.6.

In some embodiments, the anti-CD38 antibody is administered or provided for administration in a pharmaceutical composition comprising
about 1,800 mg of the anti-CD38 antibody;
about 30,000 U of rHuPH20;
about 10 mM histidine;
about 300 mM sorbitol;
about 0.04% (w/v) PS-20; and
about 1 mg/mL methionine, at a pH of about 5.6.

In some embodiments, the anti-CD38 antibody is administered or provided for administration in a pharmaceutical composition comprising
about 120 mg/mL of the anti-CD38 antibody;
about 2,000 U/mL of rHuPH20;
about 10 mM histidine;
about 300 mM sorbitol;
about 0.04% (w/v) PS-20; and
about 1 mg/mL methionine, at a pH of about 5.6.

The dose of the BCMAxCD3 bispecific antibody and the anti-CD38 antibody given to a subject having cancer, such as multiple myeloma, is sufficient to alleviate or at least partially arrest the disease being treated (“therapeutically effective amount”) and includes from about 0.005 mg to about 100 mg/kg, e.g. about 0.05 mg to about 30 mg/kg or about 5 mg to about 25 mg/kg, or about 4 mg/kg, about 8 mg/kg, about 16 mg/kg, or about 24 mg/kg of the antibody. Suitable doses include, for example, about 1, 2, 3, 4, 5, 6, 7, 8, 9, 10, 15, 16, 17, 18, 19, 20, 21, 22, 23, 24, 25, 30, 40, 50, 60, 70, 80, 90, or 100 mg/kg.

A fixed unit dose of the BCMAxCD3 bispecific antibody and/or the anti-CD38 antibody may also be given, for example, 50, 100, 200, 500, or 1000 mg, or the dose may be based on the patient's surface area, e.g., 500, 400, 300, 250, 200, or 100 mg/m². Usually between 1 and 8 doses, (e.g., 1, 2, 3, 4, 5, 6, 7, or 8) may be administered to treat a cancer, such as a multiple myeloma, but 9, 10, 11, 12, 13, 14, 15, 16, 17, 18, 19, 20, or more doses may be given.

The administration of the BCMAxCD3 bispecific antibody and/or the anti-CD38 antibody may be repeated after one day, two days, three days, four days, five days, six days, one week, two weeks, three weeks, one month, five weeks, six weeks, seven weeks, two months, three months, four months, five months, six months, or longer. Repeated courses of treatment are also possible, as is chronic administration. The repeated administration may be at the same dose or at a different dose. For example, the BCMAxCD3 bispecific antibody and the anti-CD38 antibody may be administered at 8 mg/kg or at 16 mg/kg at weekly intervals for 8 weeks, followed by administration at 8 mg/kg or at 16 mg/kg every two weeks for an additional 16 weeks, followed by administration at 8 mg/kg or at 16 mg/kg every four weeks by intravenous infusion.

The BCMAxCD3 bispecific antibody and the anti-CD38 antibody may be administered by maintenance therapy, such as, e.g., once a week for a period of 6 months or more. For example, the BCMAxCD3 bispecific antibody and the anti-CD38 antibody may be provided as a daily dosage in an amount of about 0.1 mg/kg to about 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90, or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19, or 20 after initiation of treatment, or any combination thereof, using single or divided doses of every 24, 12, 8, 6, 4, or 2 hours, or any combination thereof.

The BCMAxCD3 bispecific antibody and the anti-CD38 antibody may also be administered prophylactically in order to reduce the risk of developing the cancer, such as the multiple myeloma, delay the onset of the occurrence of an event in cancer progression, and/or reduce the risk of recurrence when the cancer is in remission.

In some embodiments, the BCMAxCD3 bispecific antibody is administered to the subject after the subject has been administered the anti-CD38 antibody. The BCMAxCD3 bispecific antibody may be administered one week, two weeks, three weeks, one month, five weeks, six weeks, seven weeks, two months, three months, four months, five months, six months, or longer after administering the anti-CD38 antibody. In some embodiments, the subject administered the BCMAxCD3 antibody is resistant and/or refractory to treatment with the anti-CD38 antibody.

The invention also provides pharmaceutical composition comprising a BCMAxCD3 bispecific antibody comprising a BCMA binding domain comprising a VH of SEQ ID NO: 29 and a VL of SEQ ID NO: 30 and a CD3 binding domain comprising the VH of SEQ ID NO: 39 and the VL of SEQ ID NO: 40, and an anti-CD38 antibody comprising a VH of SEQ ID NO: 4 and the VL of SEQ ID NO: 5.

In some embodiments, the pharmaceutical composition comprises the BCMAxCD3 bispecific antibody comprising the HC1 of SEQ ID NO: 31, the LC1 of SEQ ID NO: 32, the HC2 of SEQ ID NO: 41 the LC2 of SEQ ID NO: 42, and the anti-CD38 antibody comprising the HC of SEQ ID NO: 12 and the LC of SEQ ID NO: 13.

In some embodiment, the pharmaceutical composition is a non-fixed combination.

In some embodiments, the pharmaceutical composition comprises from about 20 mg/mL to about 120 mg/mL of the anti-CD38 antibody in about 25 mM acetic acid, about 60 mM sodium chloride, about 140 mannitol and about 0.04% w/v polysorbate-20 (PS-20); at pH about 5.5.

The BCMAxCD3 bispecific antibody may be formulated as a pharmaceutical composition comprising about 20 mg/mL to about 120 mg/mL antibody, acetic acid, histidine, sodium chloride, mannitol and/or polysorbate-20.

In some embodiments, the pharmaceutical composition comprises about 1,800 mg of the anti-CD38 antibody and about 30,000 U of rHuPH20.

In some embodiments, the pharmaceutical composition comprises about 120 mg/mL of the anti-CD38 antibody and about 2,000 U/mL of rHuPH20.

In some embodiments, the pharmaceutical composition further comprises one or more excipients.

In some embodiments, the one or more excipients is histidine, methionine, sorbitol or polysorbate-20 (PS-20), or any combination thereof.

In some embodiments, the pharmaceutical composition comprises
between about 100 mg/mL and about 120 mg/mL of the anti-CD38 antibody formulated in
between about 5 mM and about 15 mM histidine;
between about 100 mM and about 300 mM sorbitol;
between about 0.01% w/v and about 0.04% w/v PS-20; and
between about 1 mg/mL and about 2 mg/mL methionine, at a pH of about 5.5-5.6.

In some embodiments, the pharmaceutical composition comprises about 10 mM histidine.

In some embodiments, the pharmaceutical composition comprises about 300 mM sorbitol.

In some embodiments, the pharmaceutical composition comprises about 0.04% (w/v) PS-20.

In some embodiments, the pharmaceutical composition comprises about 1 mg/mL methionine.

In some embodiments, the pharmaceutical composition comprises
about 1,800 mg of the anti-CD38 antibody;
about 30,000 U of rHuPH20;
about 10 mM histidine;
about 300 mM sorbitol;
about 0.04% (w/v) PS-20; and
about 1 mg/mL methionine, at a pH of about 5.6.

In some embodiments, the pharmaceutical composition comprises
about 120 mg/mL of the anti-CD38 antibody;
about 2,000 U/mL of rHuPH20;
about 10 mM histidine;
about 300 mM sorbitol;
about 0.04% (w/v) PS-20; and
about 1 mg/mL methionine, at a pH of about 5.6.

The disclosure also provides a kit comprising the pharmaceutical composition comprising the BCMAxCD3 bispecific antibody and the anti-CD38 antibody.

Treatment with BCMAxCD3 Bispecific Antibodies in Relapsed or Refractory Subjects

The disclosure also provides a method of treating a cancer in a subject, comprising administering a therapeutically effective amount of a BCMAxCD3 bispecific antibody to the subject to treat the cancer, wherein the subject is relapsed or refractory to treatment with a prior anti-cancer therapeutic.

In some embodiments, the BCMAxCD3 bispecific antibody comprises a BCMA binding domain comprising the HCDR1 of SEQ ID NO: 23, the HCDR2 of SEQ ID NO: 24, the HCDR3 of SEQ ID NO: 25, the LCDR1 of SEQ ID NO: 26, the LCDR2 of SEQ ID NO: 27 and the LCDR3 of SEQ ID NO: 28, and a CD3 binding domain comprising the HCDR1 of SEQ ID NO: 33, the HCDR2 of SEQ ID NO: 34, the HCDR3 of SEQ ID NO: 35, the LCDR1 of SEQ ID NO: 36, the LCDR2 of SEQ ID NO: 37 and the LCDR3 of SEQ ID NO: 38.

In some embodiments, the BCMA binding domain comprises the VH of SEQ ID NO: 29 and the VL of SEQ ID NO: 30, and the CD3 biding domain comprises the VH of SEQ ID NO: 39 and the VL of SEQ ID NO: 40.

In some embodiments, the BCMAxCD3 bispecific antibody is an IgG4 isotype and comprises phenylalanine at position 405 and arginine at position 409 in the HC1 and leucine at position 405 and lysine at position 409 in the HC2, wherein residue numbering is according to the EU Index.

In some embodiments, the BCMAxCD3 bispecific antibody further comprises proline at position 228, alanine at position 234 and alanine at position 235 in both the HC1 and the HC2.

In some embodiments, the BCMAxCD3 bispecific antibody comprises the HC1 of SEQ ID NO: 31, the LC1 of SEQ ID NO: 32, the HC2 of SEQ ID NO: 41 and the LC2 of SEQ ID NO: 42.

In some embodiments, the cancer is a hematological malignancy.

In some embodiments, the hematological malignancy is a multiple myeloma.

In some embodiments, the multiple myeloma is a high-risk multiple myeloma.

In some embodiments, the subject having the high-risk multiple myeloma has one or more chromosomal abnormalities comprising:
t(4; 14)(p16; q32);
t(14; 16)(q32; q23);
del17p;
1qAmp;
t(4; 14)(p16; q32) and t(14; 16)(q32; q23);
t(4; 14)(p16; q32) and del17p;
t(14; 16)(q32; q23) and del17p; or
t(4; 14)(p16; q32), t(14; 16)(q32; q23) and del17p, or any combination thereof.

In some embodiments, the subject is relapsed or refractory to treatment with the anti-CD38 antibody, lenalinomide, bortezomib, pomalidomide, carfilzomib, elotozumab, ixazomib, melphalan or thalidomide, or any combination thereof.

In some embodiments, the subject is relapsed or refractory to treatment with lenalinomide. In some embodiments, the subject is relapsed or refractory to treatment with bortezomib. In some embodiments, the subject is relapsed or refractory to treatment with pomalidomide. In some embodiments, the subject is relapsed or refractory to treatment with carfilzomib. In some embodiments, the subject is relapsed or refractory to treatment with elotozumab. In some embodiments, the subject is relapsed or refractory to treatment with ixazomib. In some embodiments, the subject is relapsed or refractory to treatment with melphalan. In some embodiments, the subject is relapsed or refractory to treatment with thalidomide.

In some embodiments, the subject is relapsed to treatment with the anti-CD38 antibody.

In some embodiments, the anti-CD38 antibody comprises the HCDR1 of SEQ ID NO: 6, the HCDR2 of SEQ ID NO: 7, the HCDR3 of SEQ ID NO: 8, the LCDR1 of SEQ ID NO: 9, the LCDR2 of SEQ ID NO: 10 and the LCDR3 of SEQ ID NO: 11.

In some embodiments, the anti-CD38 antibody comprises the VH of SEQ ID NO: 4 and the VL of SEQ ID NO: 5.

In some embodiments, the anti-CD38 antibody is an IgG1 isotype.

In some embodiments, the anti-CD38 antibody comprises the HC of SEQ ID NO: 12 and the LC of SEQ ID NO: 13.

In some embodiments, the anti-CD38 antibody comprises
the VH of SEQ ID NO: 14 and the VL of SEQ ID NO: 15;
the VH of SEQ ID NO: 16 and the VL of SEQ ID NO: 17;
the VH of SEQ ID NO: 18 and the VL of SEQ ID NO: 19; or
the VH of SEQ ID NO: 20 and the VL of SEQ ID NO: 21.

In some embodiments, the anti-CD38 antibody is an IgG1 isotype.

In some embodiments, the subject is a human.

In some embodiments, the method further comprises administering to the subject one or more anti-cancer therapies.

In some embodiments, the one or more anti-cancer therapies is selected from the group consisting of an autologous stem cell transplant (ASCT), radiation, surgery, a chemotherapeutic agent, an immunomodulatory agent and a targeted cancer therapy.

In some embodiments, the one or more anti-cancer therapies is selected from the group consisting of lenalidomide, thalidomide, pomalidomide, bortezomib, carfilzomib, elotozumab, ixazomib, melphalan, prednisone or dexamethasone, or any combination thereof.

Combination Therapies with T Cell Redirecting Therapeutics that Binds GPRC5D and Anti-CD38 Antibodies

The disclosure also provides a method of treating a cancer in a subject, comprising administering a therapeutically effective amount of a T-cell redirecting therapeutic that binds GPRC5D and an anti-CD38 antibody to the subject to treat the cancer.

In some embodiments, the anti-CD38 antibody is administered to subject prior to administering the T cell redirecting therapeutic that binds GPRC5D.

In some embodiments, the subject is relapsed or refractory to treatment with a prior anti-cancer therapeutic.

In some embodiments, the cancer is a GPRC5D expressing cancer.

In some embodiments, the GPRC5D expressing cancer is a hematological malignancy or a solid tumor.

In some embodiments, the hematological malignancy is a leukemia, a lymphoma, or a multiple myeloma.

In some embodiments, the hematological malignancy is the leukemia. In some embodiments, the hematological malignancy is the lymphoma. In some embodiments, the hematological malignancy is the multiple myeloma.

In some embodiments, the solid tumor is an ovarian cancer, a lung cancer, a stomach cancer, a prostate cancer, a renal carcinoma, a liver cancer, a pancreatic cancer, a colon cancer, an oesophageal cancer, a bladder cancer, a cervical carcinoma or a malignant melanoma. GPRC5D has been disclosed to be expressed in these tumors, see, e.g Int. Pat. Publ. No. WO2018/147245.

In some embodiments, the subject is relapsed or refractory to treatment with the anti-CD38 antibody, lenalinomide, bortezomib, pomalidomide, carfilzomib, elotozumab, ixazomib, melphalan or thalidomide, or any combination thereof.

In some embodiments, the subject is relapsed or refractory to treatment with lenalinomide. In some embodiments, the subject is relapsed or refractory to treatment with bortezomib. In some embodiments, the subject is relapsed or refractory to treatment with pomalidomide. In some embodiments, the subject is relapsed or refractory to treatment with carfilzomib. In some embodiments, the subject is relapsed or refractory to treatment with elotozumab. In some embodiments, the subject is relapsed or refractory to treatment with ixazomib. In some embodiments, the subject is relapsed or refractory to treatment with melphalan. In some embodiments, the subject is relapsed or refractory to treatment with thalidomide. In some embodiments, the subject is relapsed or refractory to treatment with the anti-CD38 antibody.

In some embodiments, the multiple myeloma is a newly diagnosed multiple myeloma.

In some embodiments, the multiple myeloma is a relapsed or refractory multiple myeloma.

In some embodiments, the multiple myeloma is a high-risk multiple myeloma.

In some embodiments, the subject having the high-risk multiple myeloma has one or more chromosomal abnormalities comprising:
t(4; 14)(p16; q32);
t(14; 16)(q32; q23);
del17p;
1qAmp;
t(4; 14)(p16; q32) and t(14; 16)(q32; q23);
t(4; 14)(p16; q32) and del17p;
t(14; 16)(q32; q23) and del17p; or
t(4; 14)(p16; q32), t(14; 16)(q32; q23) and del17p, or any combination thereof.

In some embodiments, the T-cell redirecting therapeutic binds CD3, CD3 epsilon (CD3ε), CD8, K12L4, NKG2E, NKG2D, NKG2F, BTNL3, CD186, BTNL8, PD-1, CD195, or NKG2C.

In some embodiments, the T-cell redirecting therapeutic comprises a GPRC5D binding domain comprising the HCDR1 of SEQ ID NO: 43, the HCDR2 of SEQ ID NO: 44, the HCDR3 of SEQ ID NO: 45, the LCDR1 of SEQ ID NO: 46, the LCDR2 of SEQ ID NO: 47 and the LCDR3 of SEQ ID NO: 48, and a CD3 binding domain comprising the HCDR1 of SEQ ID NO: 33, the HCDR2 of SEQ ID NO: 34, the HCDR3 of SEQ ID NO: 35, the LCDR1 of SEQ ID NO: 36, the LCDR2 of SEQ ID NO: 37 and the LCDR3 of SEQ ID NO: 38.

In some embodiments, the GPRC5D binding domain comprises the VH of SEQ ID NO: 49 and the VL of SEQ ID NO: 50 and the CD3 binding domain comprises the VH of SEQ ID NO: 39 and the VL of SEQ ID NO: 40.

In some embodiments, the T-cell redirecting therapeutic that binds GPRC5C is a multispecific antibody, a CAR or a T cell expressing the CAR.

In some embodiments, the multispecific antibody is an IgG1, an IgG2, an IgG3 or an IgG4 isotype.

In some embodiments, the multispecific antibody is the IgG1 isotype. In some embodiments, the multispecific antibody is the IgG2 isotype. In some embodiments, the multispecific antibody is the IgG3 isotype. In some embodiments, the multispecific antibody is the IgG4 isotype.

In some embodiments, the multispecific antibody comprises one or more Fc substitutions that reduces binding of the multispecific antibody to a Fcγ receptor (FcγR).

In some embodiments, the one or more Fc substitutions is selected from the group consisting of F234A/L235A on IgG4, L234A/L235A on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4 and S228P/F234A/L235A/G236-deleted/G237A/P238S on IgG4, wherein residue numbering is according to the EU index.

In some embodiments, the multispecific antibody further comprises a S228P substitution.

In some embodiments, the multispecific antibody comprises one or more asymmetric substitutions in a first CH3 domain or in a second CH3 domain, or in both the first CH3 domain and the second CH3 domain.

In some embodiments, one or more asymmetric substitutions is selected from the group consisting of F450L/K409R, wild-type/F409L_R409K, T366Y/F405A, T366W/F405W, F405W/Y407A, T394W/Y407T, T394S/Y407A, T366W/T394S, F405W/T394S and T366W/T366S_L368A_Y407V, L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F and T350V_L351Y_F405A_Y407V/T350V_T366L_K392L_T394W.

In some embodiments, the multispecific antibody comprises the HC1 of SEQ ID NO: 51, the LC1 of SEQ ID NO: 52, the HC2 of SEQ ID NO: 41 and the LC2 of SEQ ID NO: 42.

In some embodiments, the anti-CD38 antibody comprises the HCDR1 of SEQ ID NO: 6, the HCDR2 of SEQ ID NO: 7, the HCDR3 of SEQ ID NO: 8, the LCDR1 of SEQ ID NO: 9, the LCDR2 of SEQ ID NO: 10 and the LCDR3 of SEQ ID NO: 11.

In some embodiments, the anti-CD38 antibody comprises the VH of SEQ ID NO: 4 and the VL of SEQ ID NO: 5.

In some embodiments, the anti-CD38 antibody is an IgG1 isotype.

In some embodiments, the anti-CD38 antibody comprises the HC of SEQ ID NO: 12 and the LC of SEQ ID NO: 13.

In some embodiments, the anti-CD38 antibody comprises
the VH of SEQ ID NO: 14 and the VL of SEQ ID NO: 15;
the VH of SEQ ID NO: 16 and the VL of SEQ ID NO: 17;
the VH of SEQ ID NO: 18 and the VL of SEQ ID NO: 19; or
the VH of SEQ ID NO: 20 and the VL of SEQ ID NO: 21.

In some embodiments, the anti-CD38 antibody is an IgG1 isotype.

In some embodiments, the anti-CD38 antibody is administered at a dose of between about 8 mg/kg and about 16 mg/kg.

In some embodiments, the T-cell redirecting therapeutic that binds GPRC5D and the anti-CD38 antibody are administered by an intravenous injection.

In some embodiments, the T-cell redirecting therapeutic that binds GPRC5D is administered by an intravenous injection and the anti-CD38 antibody is administered by a subcutaneous injection.

In some embodiments, the T-cell redirecting therapeutic that binds GPRC5D and the anti-CD38 antibody is administered by a subcutaneous injection.

In some embodiments, the subject is a human.

In some embodiments, the T cell redirecting therapeutic that binds GPRC5D is a GPRC5DxCD3 bispecific antibody.

In some embodiments, the method further comprises administering to the subject one or more anti-cancer therapies.

In some embodiments, the one or more anti-cancer therapies is selected from the group consisting of an autologous stem cell transplant (ASCT), radiation, surgery, a chemotherapeutic agent, an immunomodulatory agent and a targeted cancer therapy.

In some embodiments, the one or more anti-cancer therapies is selected from the group consisting of lenalidomide, thalidomide, pomalidomide, bortezomib, carfilzomib, elotozumab, ixazomib, melphalan, dexamethasone or prednisone.

In some embodiments, the anti-CD38 antibody is administered or provided for administration in a pharmaceutical composition comprising between about 20 mg/mL to about 120 mg/mL of the anti-CD38 antibody in about 25 mM acetic acid, about 60 mM sodium chloride, about 140 mannitol and about 0.04% w/v polysorbate-20 (PS-20); at pH about 5.5.

In some embodiments, the anti-CD38 antibody is administered or provided for administration in a pharmaceutical composition comprising about 1,800 mg of the anti-CD38 antibody and about 30,000 U of rHuPH20.

In some embodiments, the anti-CD38 antibody is administered or provided for administration in a pharmaceutical composition comprising about 120 mg/mL of the anti-CD38 antibody and about 2,000 U/mL of rHuPH20.

In some embodiments, the anti-CD38 antibody is administered or provided for administration in a pharmaceutical composition comprising
between about 100 mg/mL and about 120 mg/mL of the anti-CD38 antibody;
between about 5 mM and about 15 mM histidine;
between about 100 mM and about 300 mM sorbitol;
between about 0.01% w/v and about 0.04% w/v PS-20; and
between about 1 mg/mL and about 2 mg/mL methionine, at a pH of about 5.5-5.6.

In some embodiments, the anti-CD38 antibody is administered or provided for administration in a pharmaceutical composition comprising
about 1,800 mg of the anti-CD38 antibody;
about 30,000 U of rHuPH20;
about 10 mM histidine;
about 300 mM sorbitol;
about 0.04% (w/v) PS-20; and
about 1 mg/mL methionine, at a pH of about 5.6.

In some embodiments, the anti-CD38 antibody is administered or provided for administration in a pharmaceutical composition comprising
about 120 mg/mL of the anti-CD38 antibody;
about 2,000 U/mL of rHuPH20;
about 10 mM histidine;
about 300 mM sorbitol;
about 0.04% (w/v) PS-20; and
about 1 mg/mL methionine, at a pH of about 5.6.

The disclosure also provides a pharmaceutical combination comprising a GPRC5DxCD3 bispecific antibody comprising a GPRC5D binding domain comprising the HCDR1 of SEQ ID NO: 43, the HCDR2 of SEQ ID NO: 44, the HCDR3 of SEQ ID NO: 45, the LCDR1 of SEQ ID NO: 46, the LCDR2 of SEQ ID NO: 47 and the LCDR3 of SEQ ID NO: 48, and a CD3 binding domain comprising the HCDR1 of SEQ ID NO: 33, the HCDR2 of SEQ ID NO: 34, the HCDR3 of SEQ ID NO: 35, the LCDR1 of SEQ ID NO: 36, the LCDR2 of SEQ ID NO: 37 and the LCDR3 of SEQ ID NO: 38 and an anti-CD38 antibody comprising the HCDR1 of SEQ ID NO: 6, the HCDR2 of SEQ ID NO: 7, the HCDR3 of SEQ ID NO: 8, the LCDR1 of SEQ ID NO: 9, the LCDR2 of SEQ ID NO: 10 and the LCDR3 of SEQ ID NO: 11.

In some embodiments, the GPRC5D binding domain comprises the VH of SEQ ID NO: 49 and the VL of SEQ ID NO: 50 and the CD3 binding domain comprises the VH of SEQ ID NO: 39 and the VL of SEQ ID NO: 40, and the anti-CD38 antibody comprises the VH of SEQ ID NO: 4 and the VL of SEQ ID NO: 5.

In some embodiments, the GPRC5DxCD3 bispecific antibody comprises the HC1 of SEQ ID NO: 51, the LC1 of SEQ ID NO: 52, the HC2 of SEQ ID NO: 41 and the LC2 of SEQ ID NO: 42, and the anti-CD38 antibody comprises the HC of SEQ ID NO: 12 and the LC of SEQ ID NO: 13.

In some embodiments, the pharmaceutical combination is a non-fixed combination.

In some embodiments, the pharmaceutical combination comprises from about 20 mg/mL to about 120 mg/mL of the anti-CD38 antibody in about 25 mM acetic acid, about 60 mM sodium chloride, about 140 mannitol and about 0.04% w/v polysorbate-20 (PS-20); at pH about 5.5.

In some embodiments, the pharmaceutical combination comprises about 1,800 mg of the anti-CD38 antibody and about 30,000 U of rHuPH20.

In some embodiments, the pharmaceutical combination comprises about 120 mg/mL of the anti-CD38 antibody and about 2,000 U/mL of rHuPH20.

In some embodiments, the pharmaceutical combination further comprises one or more excipients.

In some embodiments, the one or more excipients is histidine, methionine, sorbitol or polysorbate-20 (PS-20), or any combination thereof.

In some embodiments, the pharmaceutical composition comprises
between about 100 mg/mL and about 120 mg/mL of the anti-CD38 antibody;
between about 5 mM and about 15 mM histidine;
between about 100 mM and about 300 mM sorbitol;
between about 0.01% w/v and about 0.04% w/v PS-20; and
between about 1 mg/mL and about 2 mg/mL methionine, at a pH of about 5.5-5.6.

In some embodiments, the pharmaceutical combination comprises about 10 mM histidine.

In some embodiments, the pharmaceutical combination comprises about 300 mM sorbitol.

In some embodiments, the pharmaceutical combination comprises about 0.04% (w/v) PS-20.

In some embodiments, the pharmaceutical combination comprises about 1 mg/mL methionine.

In some embodiments, the pharmaceutical combination comprises
about 1,800 mg of the anti-CD38 antibody;
about 30,000 U of rHuPH20;
about 10 mM histidine;
about 300 mM sorbitol;
about 0.04% (w/v) PS-20; and
about 1 mg/mL methionine, at a pH of about 5.6.

In some embodiments, the pharmaceutical combination comprises
about 120 mg/mL of the anti-CD38 antibody;
about 2,000 U/mL of rHuPH20;
about 10 mM histidine;
about 300 mM sorbitol;
about 0.04% (w/v) PS-20; and
about 1 mg/mL methionine, at a pH of about 5.6.

The disclosure also provides a pharmaceutical combination comprising the T cell redirecting therapeutic that binds GPRC5D and the anti-CD38 antibody.

Treatment with GPRC5DxCD3 Bispecific Antibodies in Relapsed or Refractory Subjects

The disclosure also provides a method of treating a cancer in a subject, comprising administering a therapeutically effective amount of a GPRC5DxCD3 bispecific antibody to the subject to treat the cancer, wherein the subject is relapsed or refractory to treatment with a prior anti-cancer therapeutic.

In some embodiments, the GPRC5DxCD3 bispecific antibody comprises a GPRC5D binding domain comprising the HCDR1 of SEQ ID NO: 43, the HCDR2 of SEQ ID NO: 44, the HCDR3 of SEQ ID NO: 45, the LCDR1 of SEQ ID NO: 46, the LCDR2 of SEQ ID NO: 47 and the LCDR3 of SEQ ID NO: 48, and a CD3 binding domain comprising the HCDR1 of SEQ ID NO: 33, the HCDR2 of SEQ ID NO: 34, the HCDR3 of SEQ ID NO: 35, the LCDR1 of SEQ ID NO: 36, the LCDR2 of SEQ ID NO: 37 and the LCDR3 of SEQ ID NO: 38.

In some embodiments, the GPRC5D binding domain comprises the VH of SEQ ID NO: 49 and the VL of SEQ ID NO: 50 and the CD3 binding domain comprises the VH of SEQ ID NO: 39 and the VL of SEQ ID NO: 40.

In some embodiments, the GPRC5DxCD3 bispecific antibody is an IgG4 isotype and comprises phenylalanine at position 405 and arginine at position 409 in the HC1 and leucine at position 405 and lysine at position 409 in the HC2, wherein residue numbering is according to the EU Index.

In some embodiments, the GPRC5DxCD3 bispecific antibody further comprises proline at position 228, alanine at position 234 and alanine at position 235 in both the HC1 and the HC2.

In some embodiments, the GPRC5DxCD3 bispecific antibody comprises the HC1 of SEQ ID NO: 51, the LC1 of SEQ ID NO: 52, the HC2 of SEQ ID NO: 41 and the LC2 of SEQ ID NO: 42.

In some embodiments, the cancer is a hematological malignancy or a solid tumor

In some embodiments, the cancer is a multiple myeloma, a lymphoma, a melanoma, a breast cancer, an endometrial cancer, an ovarian cancer, a lung cancer, stomach cancer, a prostate cancer, a renal carcinoma, a liver cancer, a pancreatic cancer, a colon cancer, an oesophageal cancer, a bladder cancer or a cervical carcinoma.

In some embodiments, the multiple myeloma is a high-risk multiple myeloma.

In some embodiments, the subject having the high-risk multiple myeloma has one or more chromosomal abnormalities comprising:
t(4; 14)(p16; q32);
t(14; 16)(q32; q23);
del17p;
1qAmp;
t(4; 14)(p16; q32) and t(14; 16)(q32; q23);
t(4; 14)(p16; q32) and del17p;
t(14; 16)(q32; q23) and del17p; or
t(4; 14)(p16; q32), t(14; 16)(q32; q23) and del17p, or any combination thereof.

In some embodiments, the subject is refractory or relapsed to treatment with the anti-CD38 antibody, lenalinomide, bortezomib, pomalidomide, carfilzomib, elotozumab, ixazomib, melphalan or thalidomide, or any combination thereof.

In some embodiments, the subject is relapsed or refractory to treatment with the anti-CD38 antibody.

In some embodiments, the anti-CD38 antibody comprises the HCDR1 of SEQ ID NO: 6, the HCDR2 of SEQ ID NO: 7, the HCDR3 of SEQ ID NO: 8, the LCDR1 of SEQ ID NO: 9, the LCDR2 of SEQ ID NO: 10 and the LCDR3 of SEQ ID NO: 11.

In some embodiments, the anti-CD38 antibody comprises the VH of SEQ ID NO: 4 and the VL of SEQ ID NO: 5.

In some embodiments, the anti-CD38 antibody is an IgG1 isotype.

In some embodiments, the anti-CD38 antibody comprises the HC of SEQ ID NO: 12 and the LC of SEQ ID NO: 13.

In some embodiments, the anti-CD38 antibody comprises
the VH of SEQ ID NO: 14 and the VL of SEQ ID NO: 15;
the VH of SEQ ID NO: 16 and the VL of SEQ ID NO: 17;
the VH of SEQ ID NO: 18 and the VL of SEQ ID NO: 19; or
the VH of SEQ ID NO: 20 and the VL of SEQ ID NO: 21.

In some embodiments, the anti-CD38 antibody is an IgG1 isotype.

In some embodiments, the subject is a human.

In some embodiments, the method further comprises administering to the subject one or more anti-cancer therapies.

In some embodiments, the one or more anti-cancer therapies is selected from the group consisting of an autologous stem cell transplant (ASCT), radiation, surgery, a chemotherapeutic agent, an immunomodulatory agent and a targeted cancer therapy.

In some embodiments, the one or more anti-cancer therapies is selected from the group consisting of lenalidomide, thalidomide, pomalidomide, bortezomib, carfilzomib, elotozumab, ixazomib, melphalan, dexamethasone, vincristine, cyclophosphamide, hydroxydaunorubicin, prednisone, rituximab, imatinib, dasatinib, nilotinib, bosutinib, ponatinib, bafetinib, saracatinib, tozasertib or danusertib, cytarabine, daunorubicin, idarubicin, mitoxantrone, hydroxyurea, decitabine, cladribine, fludarabine, topotecan, etoposide 6-thioguanine, corticosteroid, methotrexate, 6-mercaptopurine, azacitidine, arsenic trioxide and all-trans retinoic acid, or any combination thereof.

Combination Therapies with T Cell Redirecting Therapeutics that Bind CD19 and Anti-CD38 Antibodies

The disclosure also provides a method of treating a cancer in a subject, comprising administering a therapeutically effective amount of a T-cell redirecting therapeutic that binds CD19 and an anti-CD38 antibody to the subject to treat the cancer.

In some embodiments, the subject has been treated with an anti-CD38 antibody prior to administering the T-cell redirecting therapeutic that binds CD19.

The disclosure also provides a method of enhancing efficacy of a T cell redirecting therapeutic that binds CD19 in a subject having a cancer, comprising administering to the subject an anti-CD38 antibody prior to administering the T cell redirecting therapeutic that binds CD19.

In some embodiments, the subject is relapsed or refractory to treatment with a prior anti-cancer therapeutic.

In some embodiments, the cancer is a hematological malignancy or a solid tumor.

In some embodiments, the hematological malignancy is lymphoma, a B cell malignancy, Hodgkin's lymphoma, non-Hodgkin's lymphoma, a DLBLC, a FL, a MCL, a marginal zone B-cell lymphoma (MZL), a mucosa-associated lymphatic tissue lymphoma (MALT), a CLL, an ALL, an AML, Waldenstrom's Macroglobulinemia or a T-cell lymphoma.

In some embodiments, the solid tumor is a lung cancer, a liver cancer, a cervical cancer, a colon cancer, a breast cancer, an ovarian cancer, a pancreatic cancer, a melanoma, a glioblastoma, a prostate cancer, an esophageal cancer or a gastric cancer. WO2019057124A1 discloses cancers that are amenable to treatment with T cell redirecting therapeutics that bind CD19.

In some embodiments, the T-cell redirecting therapeutic binds CD3 epsilon (CD3ε), CD8, KI2L4, NKG2E, NKG2D, NKG2F, BTNL3, CD186, BTNL8, PD-1, CD195, or NKG2C.

In some embodiments, the T-cell redirecting therapeutic that binds CD19 comprises a CD19 binding domain of blinatumomab, axicabtagene ciloleucel, tisagenlecleucel-t, inebilizumab, lisocabtagene maraleucel, XmAb-5574, CIK-CAR.CD19, ICTCAR-011, IM-19, JCAR-014, loncastuximab tesirine, MB-CART2019.1, OXS-1550, PBCAR-0191, PCAR-019, PCAR-119, Senl-001, TI-1007, XmAb-5871, PTG-01, PZ01, Senl_1904A, Senl_1904B, UCART-19, CSG-CD19, DI-B4, ET-190, GC-007F or GC-022.

In some embodiments, the T cell redirecting therapeutic that binds CD19 comprises blinatumomab, axicabtagene ciloleucel, tisagenlecleucel-t, inebilizumab, lisocabtagene maraleucel, XmAb-5574, CIK-CAR.CD19, ICTCAR-011, IM-19, JCAR-014, loncastuximab tesirine, MB-CART2019.1, OXS-1550, PBCAR-0191, PCAR-019, PCAR-119, Senl-001, TI-1007, XmAb-5871, PTG-01, PZ01, Senl_1904A, Senl_1904B, UCART-19, CSG-CD19, DI-B4, ET-190, GC-007F or GC-022.

In some embodiments, the T-cell redirecting therapeutic that binds CD19 is a multispecific antibody, a CAR or a T cell expressing the CAR.

In some embodiments, the anti-CD38 antibody comprises the HCDR1 of SEQ ID NO: 6, the HCDR2 of SEQ ID NO: 7, the HCDR3 of SEQ ID NO: 8, the LCDR1 of SEQ ID NO: 9, the LCDR2 of SEQ ID NO: 10 and the LCDR3 of SEQ ID NO: 11.

In some embodiments, the anti-CD38 antibody comprises the VH of SEQ ID NO: 4 and the VL of SEQ ID NO: 5.

In some embodiments, the anti-CD38 antibody is an IgG1 isotype.

In some embodiments, the anti-CD38 antibody comprises the HC of SEQ ID NO: 12 and the LC of SEQ ID NO: 13.

In some embodiments, the anti-CD38 antibody comprises
the VH of SEQ ID NO: 14 and the VL of SEQ ID NO: 15;
the VH of SEQ ID NO: 16 and the VL of SEQ ID NO: 17;
the VH of SEQ ID NO: 18 and the VL of SEQ ID NO: 19; or
the VH of SEQ ID NO: 20 and the VL of SEQ ID NO: 21.

In some embodiments, the anti-CD38 antibody is an IgG1 isotype.

In some embodiments, the anti-CD38 antibody is administered at a dose of between about 8 mg/kg and about 16 mg/kg.

In some embodiments, the T-cell redirecting therapeutic that binds CD19 and the anti-CD38 antibody are administered by an intravenous injection.

In some embodiments, the T-cell redirecting therapeutic that binds CD19 is administered by an intravenous injection and the anti-CD38 antibody is administered by a subcutaneous injection.

In some embodiments, the T-cell redirecting therapeutic that binds CD19 and the anti-CD38 antibody is administered by a subcutaneous injection.

In some embodiments, the subject is a human.

In some embodiments, the T cell redirecting therapeutic that binds CD19 is a CD19xCD3 bispecific antibody.

In some embodiments, the method further comprises administering to the subject one or more anti-cancer therapies.

In some embodiments, the one or more anti-cancer therapies is selected from the group consisting of an autologous stem cell transplant (ASCT), radiation, surgery, a chemotherapeutic agent, an immunomodulatory agent and a targeted cancer therapy.

The disclosure also provides a pharmaceutical combination comprising a CD19xCD3 bispecific antibody comprising blinatumomab of SEQ ID NO: 53 an anti-CD38 antibody comprising the HCDR1 of SEQ ID NO: 6, the HCDR2 of SEQ ID NO: 7, the HCDR3 of SEQ ID NO: 8, the LCDR1 of SEQ ID NO: 9, the LCDR2 of SEQ ID NO: 10 and the LCDR3 of SEQ ID NO: 11.

In some embodiments, the anti-CD38 antibody comprises the VH of SEQ ID NO: 4 and the VL of SEQ ID NO: 5.

In some embodiments, the anti-CD38 antibody comprises the HC of SEQ ID NO: 12 and the LC of SEQ ID NO: 13.

In some embodiments, the pharmaceutical combination is a non-fixed combination.

In some embodiments, the pharmaceutical combination comprises from about 20 mg/mL to about 120 mg/mL of the anti-CD38 antibody in about 25 mM acetic acid, about 60 mM sodium chloride, about 140 mannitol and about 0.04% w/v polysorbate-20 (PS-20); at pH about 5.5.

In some embodiments, the pharmaceutical combination comprises about 1,800 mg of the anti-CD38 antibody and about 30,000 U of rHuPH20.

In some embodiments, the pharmaceutical combination comprises about 120 mg/mL of the anti-CD38 antibody and about 2,000 U/mL of rHuPH20.

In some embodiments, the pharmaceutical combination further comprises one or more excipients.

In some embodiments, the one or more excipients is histidine, methionine, sorbitol or polysorbate-20 (PS-20), or any combination thereof.

In some embodiments, the pharmaceutical combination comprises
between about 100 mg/mL and about 120 mg/mL of the anti-CD38 antibody;
between about 5 mM and about 15 mM histidine;
between about 100 mM and about 300 mM sorbitol;
between about 0.01% w/v and about 0.04% w/v PS-20; and
between about 1 mg/mL and about 2 mg/mL methionine, at a pH of about 5.5-5.6.

In some embodiments, the pharmaceutical combination comprises about 10 mM histidine.

In some embodiments, the pharmaceutical combination comprises about 300 mM sorbitol.

In some embodiments, the pharmaceutical combination comprises about 0.04% (w/v) PS-20.

In some embodiments, the pharmaceutical combination comprises about 1 mg/mL methionine.

In some embodiments, the pharmaceutical combination comprises
about 1,800 mg of the anti-CD38 antibody;
about 30,000 U of rHuPH20;
about 10 mM histidine;
about 300 mM sorbitol;
about 0.04% (w/v) PS-20; and
about 1 mg/mL methionine, at a pH of about 5.6.

In some embodiments, the pharmaceutical combination comprises
about 120 mg/mL of the anti-CD38 antibody;
about 2,000 U/mL of rHuPH20;
about 10 mM histidine;
about 300 mM sorbitol;
about 0.04% (w/v) PS-20; and
about 1 mg/mL methionine, at a pH of about 5.6.

In some embodiments, the pharmaceutical combination comprises 35 mcg of blinatumomab formulated with citric acid monohydrate (3.35 mg), lysine hydrochloride (23.23 mg), polysorbate 80 (0.64 mg), trehalose dihydrate (95.5 mg), and sodium hydroxide to adjust pH to 7.0.

In some embodiments, blinatumomab is reconstitution with 3 mL of preservative-free Sterile Water for Injection, USP.

A kit comprising the pharmaceutical combination comprising blinatumomab of SEQ ID NO: 53 an anti-CD38 antibody comprising the HCDR1 of SEQ ID NO: 6, the HCDR2 of SEQ ID NO: 7, the HCDR3 of SEQ ID NO: 8, the LCDR1 of SEQ ID NO: 9, the LCDR2 of SEQ ID NO: 10 and the LCDR3 of SEQ ID NO: 11.

### T Cell Redirecting Therapeutics

### Multispecific Antibodies

T cell redirecting therapeutic may be a multispecific molecule such as a bispecific antibody. Various multispecific and/or bispecific formats include formats described herein and recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule, or multispecific antibodies generated by arm exchange. Exemplary multispecific and/or bispecific formats include dual targeting molecules include Dual Targeting (DT)-Ig (GSK/Domantis), Two-in-one Antibody (Genentech) and mAb2 (F-Star), Dual Variable Domain (DVD)-Ig (Abbott), Ts2Ab (Medlmmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS) and Dual Affinity Retargeting Technology (Fc-DART) (MacroGenics), F(ab)2 (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech), Bispecific T Cell Engager (BITE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies. Various formats of bispecific antibodies have been described, for example in Chames and Baty (2009) Curr Opin Drug Disc Dev 12: 276 and in Nunez-Prado et al., (2015) Drug Discovery Today 20(5):588-594.

### Methods of Generating Antibodies Used in the Methods of the Invention

The antibodies used in the methods of the invention binding specific antigens may be selected de novo from, for example, a phage display library, where the phage is engineered to express human immunoglobulins or portions thereof such as Fabs, single chain antibodies (scFv), or unpaired or paired antibody variable regions (Knappik et al., J Mol Biol 296:57-86, 2000; Krebs et al., J Immunol Meth 254:67-84, 2001; Vaughan et al., Nature Biotechnology 14:309-14, 1996; Sheets et al., PITAS (USA) 95:6157-62, 1998; Hoogenboom and Winter, J Mol Biol 227:381, 1991; Marks et al., J Mol Biol 222:581, 1991). Phage display libraries expressing antibody heavy and light chain variable regions as fusion proteins with bacteriophage pIX coat protein as described in Shi et al (2010) J. Mol. Biol. 397:385-96 and Int'l Pat. Pub. No. WO2009/085462. The antibody libraries may be screened for binding to the desired antigen, such as BCMA, CD3, CD38, CD123, CD19, CD33, PSMA or TMEFF2 extracellular domain and the obtained positive clones may be further characterized and the Fabs isolated from the clone lysates, and subsequently cloned as full length antibodies. Such phage display methods for isolating human antibodies are established in the art. See for example: U.S. Pat. Nos. 5,223,409; 5,403,484; 5,571,698; 5,427,908; 5,580,717; 5,969,108; 6,172,197; 5,885,793; 6,521,404; 6,544,731; 6,555,313; 6,582,915; and 6,593,081.

T cell redirecting bispecific antibodies may be generated in vitro in a cell-free environment by introducing asymmetrical mutations in the CH3 regions of two monospecific homodimeric antibodies and forming the bispecific heterodimeric antibody from two parent monospecific homodimeric antibodies in reducing conditions to allow disulfide bond isomerization according to methods described in Intl. Pat. Publ. No. WO2011/131746. In the methods, two monospecific bivalent antibodies are engineered to have certain substitutions at the CH3 domain that promote heterodimer stability; the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol, preferably a reducing agent selected from the group consisting of: 2-mercaptoethylamine, dithiothreitol and tris(2-carboxyethyl)phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

Exemplary CH3 mutations that may be used in a first heavy chain and in a second heavy chain of the bispecific antibody are K409R and/or F405L.

Additional CH3 mutations that may be used include technologies such as Duobody® mutations (Genmab), Knob-in-Hole mutations (Genentech), electrostatically-matched mutations (Chugai, Amgen, NovoNordisk, Oncomed), the Strand Exchange Engineered Domain body (SEEDbody) (EMD Serono), and other asymmetric mutations (e.g. Zymeworks).

Duobody® mutations (Genmab) are disclosed for example in U.S. Pat. No. 9,150,663 and US2014/0303356 and include mutations F405L/K409R, wild-type/F405L_R409K, T350I_K370T_F405L/K409R, K370W/K409R, D399AFGHILMNRSTVWY/K409R, T366ADEFGHILMQVY/K409R, L368ADEGHNRSTVQ/K409AGRH, D399FHKRQ/K409AGRH, F405IKLSTVW/K409AGRH and Y407LWQ/K409AGRH.

Knob-in-hole mutations are disclosed for example in WO1996/027011 and include mutations on the interface of CH3 region in which an amino acid with a small side chain (hole) is introduced into the first CH3 region and an amino acid with a large side chain (knob) is introduced into the second CH3 region, resulting in preferential interaction between the first CH3 region and the second CH3 region. Exemplary CH3 region mutations forming a knob and a hole are T366Y/F405A, T366W/F405W, F405W/Y407A, T394W/Y407T, T394S/Y407A, T366W/T394S, F405W/T394S and T366W/T366S_L368A_Y407V.

Heavy chain heterodimer formation may be promoted by using electrostatic interactions by substituting positively charged residues on the first CH3 region and negatively charged residues on the second CH3 region as described in US2010/0015133, US2009/0182127, US2010/028637 or US2011/0123532.

Other asymmetric mutations that can be used to promote heavy chain heterodimerization are L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in US2012/0149876 or US2013/0195849.

SEEDbody mutations involve substituting select IgG residues with IgA residues to promote heavy chai heterodimerization as described in US20070287170.

Other exemplary mutations that may be used are R409D_K370E/D399K_E357K, S354C_T366W/Y349C_T366S_L368A_Y407V, Y349C_T366W/S354C_T366S_L368A_Y407V, T366K/L351D, L351K/Y349E, L351K/Y349D, L351K/L368E, L351Y_Y407A/T366A_K409F, L351Y_Y407A/T366V_K409F, K392D/D399K, K392D/E356K, K253E D282K K322D/D239K_E240K K292D, K392D_K409D/D356K_D399K as described in WO2007/147901, WO 2011/143545, WO2013157954, WO2013096291 and US2018/0118849.

Additional bispecific or multispecific structures that can be used as T cell redirecting therapeutics include Dual Variable Domain Immunoglobulins (DVD) (Int. Pat. Publ. No. WO2009/134776; DVDs are full length antibodies comprising the heavy chain having a structure VH1-linker-VH2-CH and the light chain having the structure VL1-linker-VL2-CL; linker being optional), structures that include various dimerization domains to connect the two antibody arms with different specificity, such as leucine zipper or collagen dimerization domains (Int. Pat. Publ. No. WO2012/022811, U.S. Pat. Nos. 5,932,448; 6,833,441), two or more domain antibodies (dAbs) conjugated together, diabodies, heavy chain only antibodies such as camelid antibodies and engineered camelid antibodies, Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer), IgG-like Bispecific (InnClone/Eli Lilly), Ts2Ab (Medlmmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)r-Fab (National Research Center for Antibody Medicine—China), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

Fc Engineering of Antibodies The Fc region of the T cell redirecting therapeutics such as bispecific or multispecific antibodies or the anti-CD38 antibodies may comprise at least one substitution in the Fc region that reduces binding of the T cell redirecting therapeutics to an activating Fcγ receptor (FcγR) and/or reduces Fc effector functions such as C1q binding, complement dependent cytotoxicity (CDC), antibody-dependent cell-mediated cytotoxicity (ADCC) or phagocytosis (ADCP).

Fc positions that may be substituted to reduce binding of the Fc to the activating FcγR and subsequently to reduce effector function are substitutions L234A/L235A on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4.

Fc substitutions that may be used to reduce CDC is a K322A substitution.

Well-known S228P substitution may further be made in IgG4 antibodies to enhance IgG4 stability.

An exemplary wild-type IgG1 comprises an amino acid sequence of SEQ ID NO: 103.

SEQ ID NO: 103:
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

An exemplary wild-type IgG4 comprises an amino acid sequence of SEQ ID NO: 104.

SEQ ID NO: 104:
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK

“Antibody-dependent cellular cytotoxicity”, “antibody-dependent cell-mediated cytotoxicity” or “ADCC” is a mechanism for inducing cell death that depends upon the interaction of antibody-coated target cells with effector cells possessing lytic activity, such as natural killer cells (NK), monocytes, macrophages and neutrophils via Fc gamma receptors (FcγR) expressed on effector cells. For example, NK cells express FcγRIIIa, whereas monocytes express FcγRI, FcγRII and FcγRIIIa. ADCC activity of the antibodies may be assessed using an in vitro assay using cells expressing the protein the antibody binds to as target cells and NK cells as effector cells. Cytolysis may be detected by the release of label (e.g. radioactive substrates, fluorescent dyes or natural intracellular proteins) from the lysed cells. In an exemplary assay, target cells are used with a ratio of 1 target cell to 4 effector cells. Target cells are pre-labeled with BATDA and combined with effector cells and the test antibody. The samples are incubated for 2 hours and cell lysis measured by measuring released BATDA into the supernatant. Data is normalized to maximal cytotoxicity with 0.67% Triton X-100 (Sigma Aldrich) and minimal control determined by spontaneous release of BATDA from target cells in the absence of any antibody.

“Antibody-dependent cellular phagocytosis” (“ADCP”) refers to a mechanism of elimination of antibody-coated target cells by internalization by phagocytic cells, such as macrophages or dendritic cells. ADCP may be evaluated by using monocyte-derived macrophages as effector cells and cells that express the protein the antibody binds to as target cells also engineered to express GFP or another labeled molecule. In an exemplary assay, effector:target cell ratio may be for example 4:1. Effector cells may be incubated with target cells for 4 hours with or without the antibody of the invention. After incubation, cells may be detached using accutase. Macrophages may be identified with anti-CD11b and anti-CD14 antibodies coupled to a fluorescent label, and percent phagocytosis may be determined based on % GFP fluorescence in the CD11⁺CD14⁻ macrophages using standard methods.

“Complement-dependent cytotoxicity”, or “CDC”, refers to a mechanism for inducing cell death in which the Fc effector domain of a target-bound antibody binds and activates complement component C1q which in turn activates the complement cascade leading to target cell death. Activation of complement may also result in deposition of complement components on the target cell surface that facilitate CDC by binding complement receptors (e.g., CR3) on leukocytes. CDC of cells may be measured for example by plating Daudi cells at 1×10⁵ cells/well (50 μL/well) in RPMI-B (RPMI supplemented with 1% BSA), adding 50 μL of test antibodies to the wells at final concentration between 0-100 μg/mL, incubating the reaction for 15 min at room temperature, adding 11 μL of pooled human serum to the wells, and incubation the reaction for 45 min at 37° C. Percentage (%) lysed cells may be detected as % propidium iodide stained cells in FACS assay using standard methods.

Binding of the antibody to FcγR or FcRn may be assessed on cells engineered to express each receptor using flow cytometry. In an exemplary binding assay, 2×10⁵ cells per well are seeded in 96-well plate and blocked in BSA Stain Buffer (BD Biosciences, San Jose, USA) for 30 min at 4° C. Cells are incubated with a test antibody on ice for 1.5 hour at 4° C. After being washed twice with BSA stain buffer, the cells are incubated with R-PE labeled anti-human IgG secondary antibody (Jackson Immunoresearch Laboratories) for 45 min at 4° C. The cells are washed twice in stain buffer and then resuspended in 150 μL of Stain Buffer containing 1:200 diluted DRAQ7 live/dead stain (Cell Signaling Technology, Danvers, USA). PE and DRAQ7 signals of the stained cells are detected by Miltenyi MACSQuant flow cytometer (Miltenyi Biotec, Auburn, USA) using B2 and B4 channel, respectively. Live cells are gated on DRAQ7 exclusion and the geometric mean fluorescence signals are determined for at least 10,000 live events collected. FlowJo software (Tree Star) is used for analysis. Data is plotted as the logarithm of antibody concentration versus mean fluorescence signals. Nonlinear regression analysis is performed.

### Chimeric Antigen Receptors (CAR)

Chimeric antigen receptors (CARs) are genetically engineered receptors. These engineered receptors can be readily inserted into and expressed by immune cells, including T cells in accordance with techniques known in the art. With a CAR, a single receptor can be programmed to both recognize a specific antigen and, when bound to that antigen, activate the immune cell to attack and destroy the cell bearing that antigen. When these antigens exist on tumor cells, an immune cell that expresses the CAR can target and kill the tumor cell.

CAR typically comprises an extracellular domain that binds the antigen (e.g. prostate neoantigen), an optional linker, a transmembrane domain, and a cytosolic domain comprising a costimulatory domain and/or a signaling domain.

The extracellular domain of CAR may contain any polypeptide that binds the desired antigen (e.g. prostate neoantigen). The extracellular domain may comprise a scFv, a portion of an antibody or an alternative scaffold. CARs may also be engineered to bind two or more desired antigens that may be arranged in tandem and separated by linker sequences. For example, one or more domain antibodies, scFvs, llama VHH antibodies or other VH only antibody fragments may be organized in tandem via a linker to provide bispecificity or multispecificity to the CAR.

The transmembrane domain of CAR may be derived from the transmembrane domain of CD8, an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDI 1a, CD18), ICOS (CD278), 4-1 BB (CD137), 4-1 BBL, GITR, CD40, BAFFR, HVEM (LIGHTR), SLAMF7, NKp80 (KLRFI), CD160, CD1 9, IL2R beta, IL2R gamma, IL7R a, ITGA1, VLA1, CD49a, ITGA4, IA4, CD49D, ITGA6, VLA-6, CD49f, ITGAD, CDI Id, ITGAE, CD103, ITGAL, CDI 1a, LFA-1, ITGAM, CDI 1b, ITGAX, CDI 1c, ITGB1, CD29, ITGB2, CD1 8, LFA-1, ITGB7, TNFR2, DNAM1 (CD226), SLAMF4 (CD244, 2B4), CD84, CD96 (Tactile), CEACAMI, CRT AM, Ly9 (CD229), CD160 (BY55), PSGL1, CD100 (SEMA4D), SLAMF6 (NTB-A, Ly108), SLAM (SLAMFI, CD150, IPO-3), BLAME (SLAMF8), SELPLG (CD162), LTBR, PAG/Cbp, NKp44, NKp30, NKp46, NKG2D, and/or NKG2C.

The intracellular costimulatory domain of CAR may be derived from the intracellular domains of one or more co-stimulatory molecules. Co-stimulatory molecules are well-known cell surface molecules other than antigen receptors or Fc receptors that provide a second signal required for efficient activation and function of T lymphocytes upon binding to antigen. Exemplary co-stimulatory domains that can be used in CARs are intracellular domains of 4-1BB, CD2, CD7, CD27, CD28, CD30, CD40, CD54 (ICAM), CD83, CD134 (OX40), CD150 (SLAMFI), CD152 (CTLA4), CD223 (LAG3), CD270 (HVEM), CD278 (ICOS), DAP10, LAT, NKD2C SLP76, TRIM, and ZAP70.

The intracellular signaling domain of CAR may be derived from the signaling domains of for example OO3ζ, CD3ε, CD22, CD79a, CD66d or CD39. “Intracellular signaling domain,” refers to the part of a CAR polypeptide that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited following antigen binding to the extracellular CAR domain.

The optional linker of CAR positioned between the extracellular domain and the transmembrane domain may be a polypeptide of about 2 to 100 amino acids in length. The linker can include or be composed of flexible residues such as glycine and serine so that the adjacent protein domains are free to move relative to one another. Longer linkers may be used when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof. The linker may also be derived from a hinge region or portion of the hinge region of any immunoglobulin.

Exemplary CARs that may be used are for example CAR that contains an extracellular domain that binds the prostate neoantigen of the invention, CD8 transmembrane domain and CD3ζ signaling domain. Other exemplary CARs contain an extracellular domain that binds the prostate neoantigen of the invention, CD8 or CD28 transmembrane domain, CD28, 41BB or OX40 costimulatory domain and CD3ζ signaling domain.

CARs are generated by standard molecular biology techniques. The extracellular domain that binds the desired antigen may be derived from antibodies or their antigen binding fragments generated using the technologies described herein.

While having described the invention in general terms, the embodiments of the invention will be further disclosed in the following examples that should not be construed as limiting the scope of the claims.

### EXAMPLES

The following examples are provided to further describe some of the embodiments disclosed herein. The examples are intended to illustrate, not to limit, the disclosed embodiments.

### General Materials and Methods

### Antibodies and Reagents

Anti-BCMA/anti-CD3 antibody JNJ-957 (described in WO2017031104A1) and daratumumab were made by Janssen Pharmaceuticals. CNTO7008 (CD3xnull), BC3B4 (BCMAxnull) and 3930 (IgG isotype control), all made by Janssen Pharmaceuticals, were used as control antibodies. JNJ-957 is also called JNJ-7957.

JNJ-957 comprises a BCMA binding arm BCMB69 and a CD3 binding arm CD3B219, the amino acid sequences of which are shown in Table 3 and Table 4, respectively.

TABLE 3

| | Region | Sequence | SEQ ID NO: |
|---|---|---|---|
| BCMB69 | HCDR1 | SGSYFWG | 23 |
| | HCDR2 | SIYYSGITYYNPSLKS | 24 |
| | HCDR3 | HDGAVAGLFDY | 25 |
| | LCDR1 | GGNNIGSKSVH | 26 |
| | LCDR2 | DDSDRPS | 27 |
| | LCDR3 | QVWDSSSDHVV | 28 |
| | VH | QLQLQESGPGLVKPSETLSLTCTVSGGSISSGSYFWG | 29 |
| | | WIRQPPGKGLEWIGSIYYSGITYYNPSLKSRVTISVD | |
| | | TSKNQFSLKLSSVTAADTAVYYCARHDGAVAGLFD | |
| | | YWGQGTLVTVSS | |
| | VL | SYVLTQPPSVSVAPGQTARITCGGNNIGSKSVHWYQ | 30 |
| | | QPPGQAPVVVVYDDSDRPSGIPERFSGSNSGNTATL | |
| | | TISRVEAGDEAVYYCQVWDSSSDHVVFGGGTKLTV | |
| | | LGQP | |
| | HC | QLQLQESGPGLVKPSETLSLTCTVSGGSISSGSYFWG | 31 |
| | | WIRQPPGKGLEWIGSIYYSGITYYNPSLKSRVTISVD | |
| | | TSKNQFSLKLSSVTAADTAVYYCARHDGAVAGLFD | |
| | | YWGQGTLVTVSSASTKGPSVFPLAPCSRSTSESTAA | |
| | | LGCLVKDYFPEPVTVSWNSGALTSGVHTFPAVLQS | |
| | | SGLYSLSSVVTVPSSSLGTKTYTCNVDHKPSNTKVD | |
| | | KRVESKYGPPCPPCPAPEAAGGPSVFLFPPKPKDTL | |
| | | MISRTPEVTCVVVDVSQEDPEVQFNWYVDGVEVH | |
| | | NAKTKPREEQFNSTYRVVSVLTVLHQDWLNGKEY | |
| | | KCKVSNKGLPSSIEKTISKAKGQPREPQVYTLPPSQE | |
| | | EMTKNQVSLTCLVKGFYPSDIAVEWESNGQPENNY | |
| | | KTTPPVLDSDGSFFLYSRLTVDKSRWQEGNVFSCSV | |
| | | MHEALHNHYTQKSLSLSLGK | |
| | LC | SYVLTQPPSVSVAPGQTARITCGGNNIGSKSVHWYQ | 32 |
| | | QPPGQAPVVVVYDDSDRPSGIPERFSGSNSGNTATL | |
| | | TISRVEAGDEAVYYCQVWDSSSDHVVFGGGTKLTV | |
| | | LGQPKAAPSVTLFPPSSEELQANKATLVCLISDFYPG | |
| | | AVTVAWKGDSSPVKAGVETTTPSKQSNNKYAASS | |
| | | YLSLTPEQWKSHRSYSCQVTHEGSTVEKTVAPTECS | |

TABLE 4

| | Region | Sequence | SEQ ID NO: |
|---|---|---|---|
| CD3B219 | HCDR1 | TYAMN | 33 |
| | HCDR2 | RIRSKYNNYATYYAASVKG | 34 |
| | HCDR3 | HGNFGNSYVSWFAY | 35 |
| | LCDR1 | RSSTGAVTTSNYAN | 36 |
| | LCDR2 | GTNKRAP | 37 |
| | LCDR3 | ALWYSNLWV | 38 |
| | VH | EVQLVESGGGLVQPGGSLRLSCAASGFTFNTYA | 39 |
| | | MNWVRQAPGKGLEWVARIRSKYNNYATYYAAS | |
| | | VKGRFTISRDDSKNSLYLQMNSLKTEDTAVYYC | |
| | | ARHGNFGNSYVSWFAYWGQGTLVTVSS | |
| | VL | QTVVTQEPSLTVSPGGTVTLTCRSSTGAVTTSNY | 40 |
| | | ANWVQQKPGQAPRGLIGGTNKRAPGTPARFSGS | |
| | | LLGGKAALTLSGVQPEDEAEYYCALWYSNLWV | |
| | | FGGGTKLTVLGQP | |
| | HC | EVQLVESGGGLVQPGGSLRLSCAASGFTFNTYA | 41 |
| | | MNWVRQAPGKGLEWVARIRSKYNNYATYYAAS | |
| | | VKGRFTISRDDSKNSLYLQMNSLKTEDTAVYYC | |
| | | ARHGNFGNSYVSWFAYWGQGTLVTVSSASTKG | |
| | | PSVFPLAPCSRSTSESTAALGCLVKDYFPEPVTVS | |
| | | WNSGALTSGVHTFPAVLQSSGLYSLSSVVTVPSS | |
| | | SLGTKTYTCNVDHKPSNTKVDKRVESKYGPPCP | |
| | | PCPAPEAAGGPSVFLFPPKPKDTLMISRTPEVTCV | |
| | | VVDVSQEDPEVQFNWYVDGVEVHNAKTKPREE | |
| | | QFNSTYRVVSVLTVLHQDWLNGKEYKCKVSNK | |
| | | GLPSSIEKTISKAKGQPREPQVYTLPPSQEEMTKN | |
| | | QVSLTCLVKGFYPSDIAVEWESNGQPENNYKTTP | |
| | | PVLDSDGSFLLYSKLTVDKSRWQEGNVFSCSVM | |
| | | HEALHNHYTQKSLSLSLGK | |
| | LC | QTVVTQEPSLTVSPGGTVTLTCRSSTGAVTTSNY | 42 |
| | | ANWVQQKPGQAPRGLIGGTNKRAPGTPARFSGS | |
| | | LLGGKAALTLSGVQPEDEAEYYCALWYSNLWV | |
| | | FGGGTKLTVLGQPKAAPSVTLFPPSSEELQANKA | |
| | | TLVCLISDFYPGAVTVAWKADSSPVKAGVETTTP | |
| | | SKQSNNKYAASSYLSLTPEQWKSHRSYSCQVTH | |
| | | EGSTVEKTVAPTECS | |

### Bone Marrow and Peripheral Blood Mononuclear Cells

Peripheral blood mononuclear cells (PBMCs) from healthy donors and MM patients, and bone marrow mononuclear cells (BM-MNCs) from MM patient BM aspirates were isolated by Ficoll-Hypaque density-gradient centrifugation.

### Cell Lines and Culture

The luciferase (LUC)-transduced multiple myeloma cell lines UM9, RPMI8226, U266 and MM1.S, as well as the non-transduced multiple myeloma cell lines NCI-H929 and RPMI8226, were cultured in RPMI 1640 (Invitrogen), supplemented with 10% fetal bovine serum (FBS; Lonza) and antibiotics (100 units/mL penicillin, 100 μg/ml streptomycin; both Life Technologies).

Flow cytometric analysis of bone marrow and blood samples from MM patients BM-localized MM cells were identified and analysed for cell surface marker expression levels by staining 1.0×10⁶ cells/mL with HuMax-003 (CD38) FITC (this antibody binds to an epitope distinct from the epitope bound by daratumumab, Janssen Pharmaceuticals), CD138 PE, CD56 PC7, CD45 Krome Orange (all Beckman Coulter), CD269 (BCMA) APC (Biolegend), CD274 (PD-L1) BV421 and CD19 APC-H7 (both Becton Dickinson). BM or PB immune cell subsets were identified and analysed for cell surface marker expression levels by staining 1.0×10⁶ cells/mL with CD45 Krome Orange, CD56 PC7 (both Beckman Coulter), CD14 APC-H7, CD19 APC-H7, CD3 V450, CD4 APC-H7 or PE, CD8 FITC, CD45-RA APC, CD127 PE.Cy7, CD62L PE, CD274 (PD-1) BV421, CD16 APC, HLA-DR APC-H7 (all Becton Dickinson) and CD25 PE (Dako). All BM samples were analysed within 24 hours from the time the sample was collected.

Flow cytometry was performed using a 7-laser LSRFORTESSA (Becton Dickinson). Fluorescent labeled beads (CS&T beads, Becton Dickinson) were used daily to monitor the performance of the flow cytometer and verify optical path and stream flow. This procedure enables controlled standardized results and allows the determination of long-term drifts and incidental changes within the flow cytometer. No changes were observed which could affect the results. Compensation beads were used to determine spectral overlap, compensation was automatically calculated using Diva software. Flow cytometry data were analyzed using FACS Diva software.

### Flow Cytometry-Based Ex Vivo Lysis Assays in BM-MNCs

BM-MNCs derived from MM patients containing tumor cells, but also autologous effector cells, were used in lysis assays. Sample viability at incubation was more than 98%, as assessed by using 7-AAD (Becton Dickinson). For lysis assays, BM-MNCs were incubated in RPMI+10% fetal bovine serum with control antibody or JNJ-957 (0.0064-4.0 μg/mL) and/or daratumumab (10 μg/mL) in 96-well U-bottom plates for 48 hours. The survival of primary CD138⁺ MM cells in the BM-MNCs was determined by flow cytometry as previously described (van der Veers et al., Haematologica. 2011; 96(2):284-290; van der Veer M S et al., Blood Cancer J. 2011; 1(10):e41; Nijhof I S et al., Leukemia 2015; 29(10):2039-2049; Nijhof I S, et al., Blood 2016; 128(7):959-970). In both assays, surviving MM cells were enumerated by single platform flow cytometric analysis of CD138⁺ cells in the presence of Flow-Count Fluorospheres (Beckman Coulter) and LIVE/DEAD Fixable Dead Cell Stain Near-IR fluorescent reactive dye (Invitrogen) to determine absolute numbers of viable MM cells. The percentage of lysis induced by JNJ-957 was then calculated using the following formula: % lysis MM cells=1−(absolute number of surviving CD138⁺ cells in the presence of JNJ-957/absolute number of surviving CD138⁺ cells in untreated wells)×100%.

The JNJ-957-induced activation and degranulation of CD4⁺ and CD8⁺ T-cells were analyzed by the flow cytometric detection of CD25 and CD107a cell surface expression, respectively.

Flow Cytometry-Based Lysis Assay in MM Cell Lines with PB MNCs as Effector Cells.

BCMA-positive MM cell lines were co-cultured with PB MNCs from healthy donors or MM patients at an effector to target ratio of 9:1 in 96-wells U-bottom plates in the presence of control antibodies or JNJ-957 (0.00256-4.0 μg/mL) for 48 hours. The survival of MM cells was determined by flow cytometry as described above.

### Bioluminescence Imaging (BLI)-Based Lysis Assay Using LUC-Transduced MM Cell Lines

LUC-transduced MM cell lines were cultured in the presence or absence of pooled BM stromal cells (BMSCs) obtained from newly diagnosed MM patient (n=12) for 16 hours prior to incubation with effector cells (freshly isolated PBMCs from healthy donors) at an effector to target ratio of 9:1, and serial dilutions of JNJ-957 (0.00256-4.0 μg/mL) or control antibodies in 96-well flat bottom plates (Greiner-Bio-One) for 48 hours. The survival of LUC*-MM cells was then determined by BLI, 10 minutes after addition of the substrate luciferin (150 μg/mL; Promega). Lysis of MM cells was determined using the following formula: % lysis=1−(mean BLI signal in the presence of effector cells and JNJ-957/mean BLI signal in the presence of effector cells in untreated wells)×100%.

To evaluate the effect of in vivo pretreatment of PB MNCs with daratumumab monotherapy on efficacy of JNJ-957, the LUC-transduced MM cell line 4 was also co-cultured with PB MNCs, obtained from MM patients before initiation of daratumumab monotherapy and at the time of best response to daratumumab monotherapy (effector to target ratio of 9:1). The BLI assay was performed as described before.

### Cytogenetic Analysis

Cytogenetic abnormalities were assessed in purified MM cells by fluorescence in situ hybridization (FISH) and single nucleotide polymorphism (SNP) array. High-risk disease was defined by the presence of del(17p), del(1p), ampl(1q), t(4; 14) or t(14; 16)².

### Soluble BCMA Assay

Soluble BCMA (sBCMA) was measured in cell culture supernatants using MSD GOLD™ 96-well Small Spot Streptavidin SECTOR plates (Meso Scale Diagnostics), according to the manufacturer's recommended protocol.

### Granzyme B Assay

Granzyme B was measured in cell culture supernatants using MSD R-Plex Granzyme B assay plates (Meso Scale Diagnostics), according to the manufacturer's protocol.

### Multiplex Cytokine Assay

Cytokines [interferon-gamma (IFN-γ), interleukin (IL)-2, IL-6, IL-8, IL-10, and tumor necrosis factor-alpha (TNF-α)] in the cell culture supernatants were analyzed using V-Plex proinflammatory Panel 1 Human Kit (Meso Scale Diagnostics), according to the manufacturer's protocol.

### Statistics

Comparisons between variables were performed using two-tailed (paired) Student's t-test, or Mann-Whitney U test or Wilcoxon matched-pairs signed-rank test in case the data do not follow a normal distribution. Correlations between variables were made using the Spearman's rank correlation coefficient. P-values below 0.05 were considered significant. In case of combinatorial treatment of JNJ-957 and daratumumab, the expected lysis values were calculated to test the null hypothesis that there is only an additive effect between JNJ-957 and daratumumab, using the following formula: % expected lysis=(% lysis with JNJ-957+% lysis with daratumumab)−(% lysis with JNJ-957 x % lysis with daratumumab), as described before^{20,23,24}. The null hypothesis of “additive effects” was rejected, if the observed values were significantly higher (P<0.05) than the expected values.

### Example 1 Anti-BCMA/Anti-CD3 Antibody JNJ-957-Mediated Lysis of BCMA⁺ Multiple Myeloma Cell Lines is Accompanied by T-Cell Activation and Degranulation

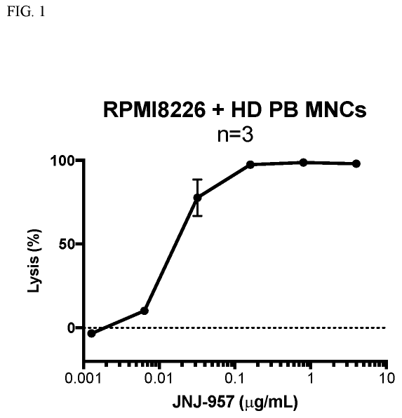

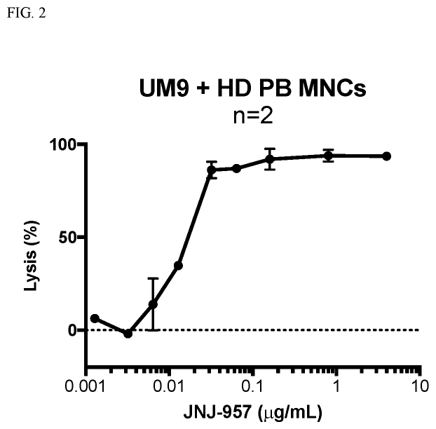

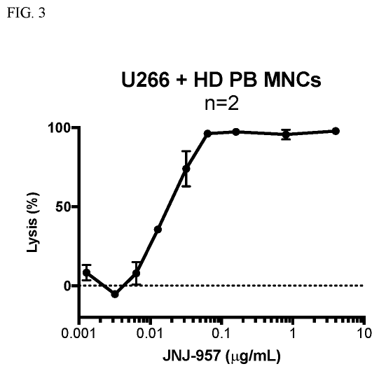

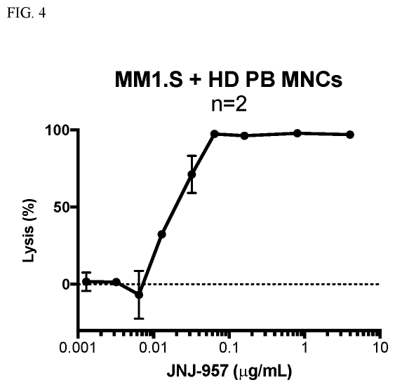

Effect of JNJ-957 on mediating lysis of RPMI8226 (FIG. 1), UM9 (FIG. 2), U226 (FIG. 3) and MM1.S (FIG. 4) multiple myeloma cell lines was assessed using healthy donor (HD) peripheral blood mononuclear cells as effector cells over a concentration range of JNJ-957 (0.00128-4.0 μg/mL). JNJ-957 mediated lysis of all tested cell lines in a dose-dependent manner and achieved nearly 100% maximal efficacy at antibody concentration of about 0.1 μg/ml, depending on the cell line as seen in FIG. 1, FIG. 2, FIG. 3 and FIG. 4.

It has previously been shown that BMSCs protect MM cells against various anti-MM agents including daratumumab and MM-reactive T-cells. The potential impact of BMSC-MM cell interactions on the efficacy of JNJ-957 was therefore assessed. The activity of JNJ-957 against the MM cell lines RPMI-8226, UM9 and U266 was not affected by the presence of BMSCs (data not shown). Although JNJ-957-mediated MM cell lysis was modestly inhibited by BMSCs in MM1.S cells at lower concentrations (P<0.0001), this effect was completely abrogated by increasing the JNJ-7957 dose.

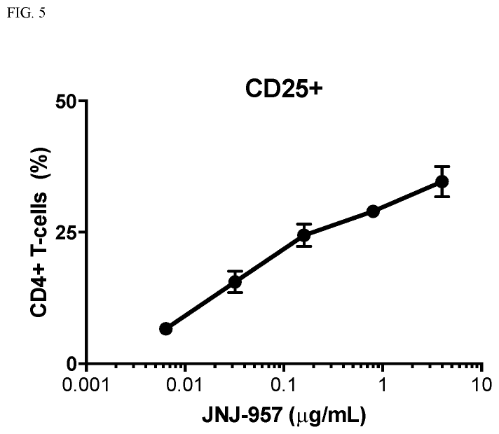

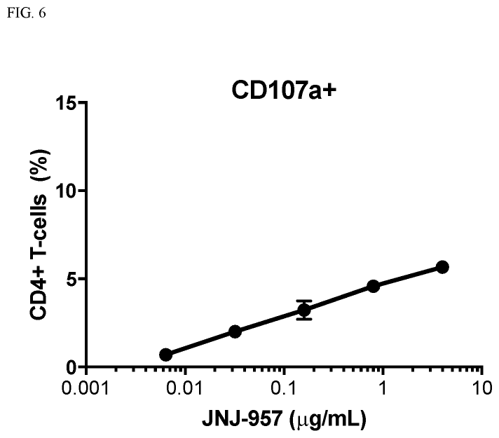

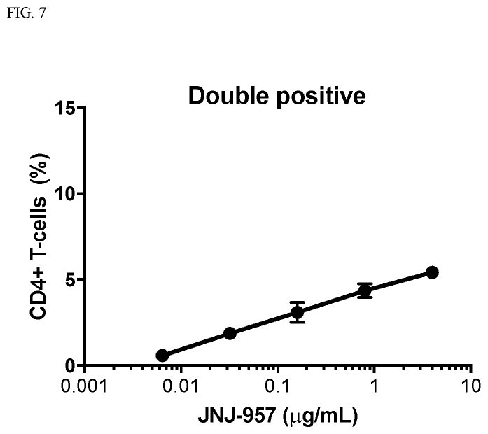

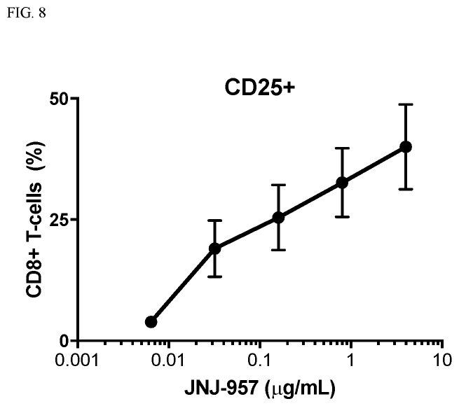

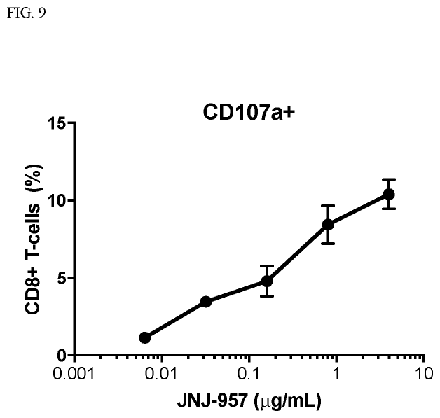

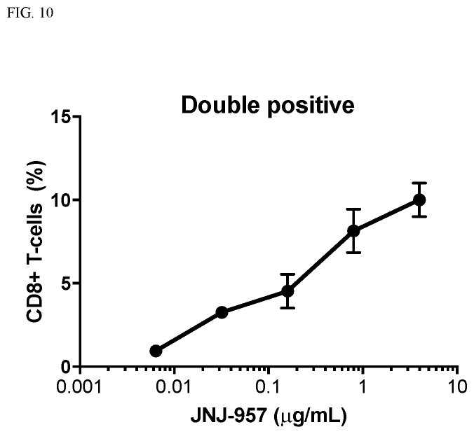

T cell activation was assessed in RPMI 8226 cell line. Treatment with JNJ-957 resulted in activation and degranulation of both CD4⁺ and CD8⁺ Tcells in a dose dependent manner, as evidenced by increased cell surface expression of CD25 and CD107a, respectively, or by the proportion of double positive CD25 and CD107a cells. FIG. 5 shows JNJ-957-mediated increase in the percentage of CD25+ CD4 T cells. FIG. 6 shows JNJ-957-mediated increase in the percentage of CD107a+ CD4 T cells. FIG. 7 shows JNJ-957-mediated increase in the percentage of the double positive CD25+ CD107+CD4 T cells. FIG. 8 shows JNJ-957-mediated increase in the percentage of CD25+ CD8 T cells. FIG. 9 shows JNJ-957-mediated increase in the percentage of CD107a+ CD8 T cells. FIG. 10 shows JNJ-957-mediated increase in the percentage of the double positive CD25+ CD107+CD8 T cells.

### Example 2 Daratumumab Improved Efficacy of T Cell Redirecting Antibodies Patients

BCMA expression levels, composition of immune cells subsets, and ex vivo efficacy of JNJ-957, were assessed in 55 BM aspirates obtained from 11 newly diagnosed MM patients, 21 daratumumab-naïve relapsed/refractory MM patients, and 17 daratumumab-refractory relapsed/refractory MM patients (daratumumab relapsed/refractory patients were enrolled in Phase 1 and Phase 2 study of daratumumab in combination with all-trans retinoic acid (ATRA); clinical trial identifier NCT02751255) and primary plasma cell leukemia (pPCL; n=6). Sequential BM samples were obtained from 8 patients treated in the DARA/ATRA study, directly before initiation of daratumumab monotherapy and at the time of progressive disease during daratumumab treatment. In the same study, we obtained from 10 patients sequential peripheral blood samples, directly before initiation of daratumumab monotherapy and at the time of maximum response achieved with daratumumab.

In the DARA/ATRA study (NCT02751255), patients had MM requiring systemic treatment and were relapsed from or refractory to ≥2 prior lines of therapy. Patients were ≥18 years of age, had a life expectancy of ≥3 months, a WHO performance status of ≤2 and measurable disease.

During the first phase of the study, daratumumab was given according to the recommended dose and schedule (16 mg/kg weekly for 8 weeks, then every 2 weeks for 16 weeks, and every 4 weeks until PD). Study site ethics committees or institutional review boards approved the protocols, which were conducted according to the principles of the Declaration of Helsinki, the International Conference on Harmonization, and the Guidelines for Good Clinical Practice. All patients gave written informed consent.

Baseline characteristics of patients enrolled in Phase 1 and Phase 2 study NCT02751255 is shown in Table 5 and Table 6. RRMM patients had received on average 5 (range 1-9) previous lines of therapies and RRMM dara R patients had received on average 6 (range 3-12) previous lines of therapies. Table 7 shows an updated summary of baseline characteristics of patients enrolled in Phase 1 and Phase 2 study.

TABLE 5

| | NDMM | RRMM | RRMM dara R |
|---|---|---|---|
| | n = 11 | n = 19 | n = 15 |
| Age, median | 66 | 66 | 68 |
| (range) | (31-80) | (46-77) | (48-80) |
| Sex, male n (%) | 5 (46) | 11 (58) | 9 (60) |
| M-protein, n (%) | | | |
| IgG | 5 (46) | 13 (68) | 11 (73) |
| IgA | 0 | 0 | 2 (13) |
| FLC only | 6 (55) | 6 (32) | 2 (13) |

NDMM: newly diagnosed multiple myeloma
RRMM: relapsed/refractory multiple myeloma
RRMM: daraR daratumumab refractory multiple myeloma

TABLE 6

| | RRMM | | RRMM dara R | |
|---|---|---|---|---|
| | n = 19 | | n = 15 | |
| Previous lines, | 5 (1-9) | | 6 (3-12) | |
| n (range) | | | | |
| | Exposed | Refractory | Exposed | Refractory |
| | n (%) | n (%) | n (%) | n (%) |
| Lenalidomide | 16 (84) | 16 (84) | 15 (100) | 15 (100) |
| Bortezomib | 14 (74) | 14 (74) | 14 (93) | 9 (60) |
| Pomalidomide | 12 (63) | 12 (63) | 10 (67) | 10 (67) |
| Carfilzomib | 5 (21) | 4 (21) | 4 (26) | 4 (26) |
| Daratumumab | 0 | 0 | 15 (100) | 15 (100) |

TABLE 7

| | | | RRMM | |
|---|---|---|---|---|
| | | RRMM | patients, | |
| | | patients, | dara- | |
| | NDMM | dara-naïve | refractory | pPCL |
| Parameter | n = 11 | n = 21 | n = 17 | n = 6 |
| Median age, years (range) | 66 (31-80) | 66 (46-77) | 68 (48-80) | 65 (57-98) |
| Sex, male, n (%) | 5 (45) | 11 (52) | 9 (53) | 2 (33) |
| M-protein type | | | | |
| IgG, n (%) | 5 (45) | 15 (71) | 13 (76) | 2 (33) |
| IgA, n (%) | 0 | 1 (5) | 2 (12) | 0 |
| FLC only, n (%) | 6 (55) | 5 (24) | 2 (12) | 3 (50) |
| Unknown | 0 | 0 | 0 | 1 (17) |
| Cytogenetics, n (%) | | | | |
| High risk* | 5 (45) | 12 (57) | 9 (53) | 3 (50) |
| Standard risk | 5 (45) | 7 (33) | 5 (29) | 1 (17) |
| Not assessed | 1 (9) | 2 (10) | 3 (18) | 2 (33) |
| Previous lines of therapy, n | 0 | 3 (1-9) | 6 (3-12) | 0 |
| (range) | | | | |
| Most recent treatment | | | | |
| No treatment | 11 (100) | 0 | 0 | 6 (100) |
| PI based | 0 | 2 (10) | 0 | 0 |
| IMiD based | 0 | 15 (71) | 1 (6)# | 0 |
| PI + IMiD | 0 | 4 (19) | 1 (6)# | 0 |
| Daratumumab | 0 | 0 | 15 (88) | 0 |
| Lenalidomide | n.a. | | | n.a. |
| exposed, n (%) | | 19 (90)§ | 17 (100) | |
| refractory**, n (%) | | 18 (86) | 17 (100) | |
| Bortezomib | n.a. | | | n.a. |
| exposed, n (%) | | 17 (81)† | 16 (94)‡ | |
| refractory**, n (%) | | 10 (48) | 11 (65) | |
| Pomalidomide refractory**, n (%) | n.a. | 13 (62) | 10 (59) | n.a. |
| Carfilzomib refractory**, n (%) | n.a. | 4 (19) | 4 (24) | n.a. |
| Daratumumab refractory**, n (%) | n.a. | 0 | 17 (100) | n.a. |
| Elotuzumab refractory**, n (%) | n.a. | 2 (10) | 1 (6) | n.a. |
| Ixazomib refractory**, n (%) | n.a. | 1 (5) | 1 (6) | n.a. |

*High-risk disease was defined by the presence of del(17p), del(1p), ampl(1q), t(4;14) or t(14;16).
**Refractory disease is defined as progressive disease during therapy, no response (less than PR), or progressive disease within 60 days of stopping treatment, according to the International Uniform Response Criteria for Multiple Myeloma.
#BM aspirates were obtained immediately at the time of development of progressive disease during daratumumab monotherapy (n = 15), while 2 BM samples were obtained 22 and 48 months after development of progression during daratumumab monotherapy, after 3 and 5 other lines of treatment, respectively.
§Additionally, 1 out of 19 patients was lenalidomide intolerant;
†Additionally, 4 out of 17 patients were bortezomib intolerant;
‡Additionally, 3 out of 16 patients were bortezomib intolerant;
Abbreviations:
MM, multiple myeloma;
NDMM, newly diagnosed MM;
RRMM, relapsed/refractory MM;
Dara, daratumumab;
pPCL, primary plasma cell leukemia;
n, number;
IgG, immunoglobulin G;
IgA, immunoglobulin A;
FLC, free light chain;
del, deletion;
amp, amplification;
t, translocation;
PI, proteasome inhibitor;
IMiD, immunomodulatory drug;

### Results

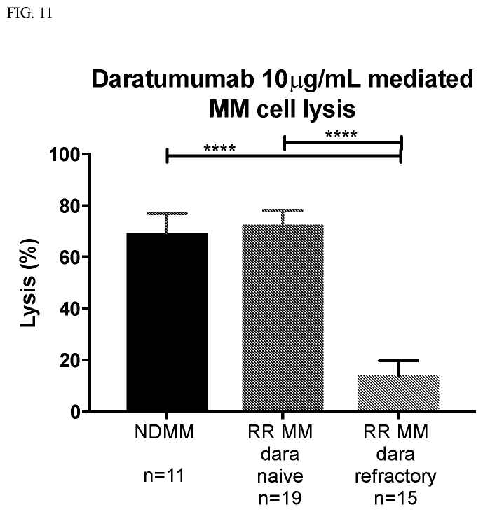

Daratumumab mediated efficient lysis of MM cells from newly diagnosed (NDMM) and relapsed/refractory daratumumab naïve patients while cells from RRMM daratumumab refractory patients were resistant to lysis (FIG. 11).

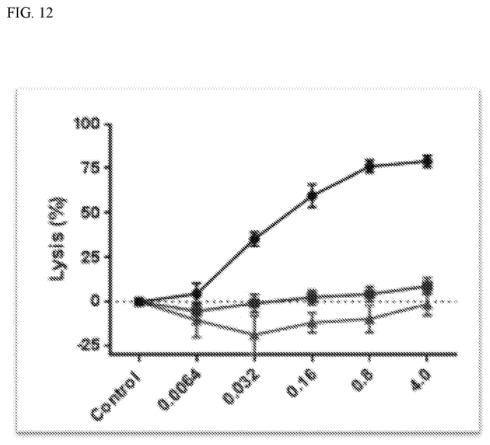

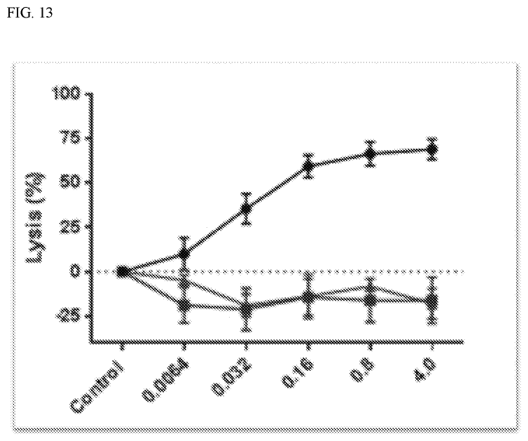

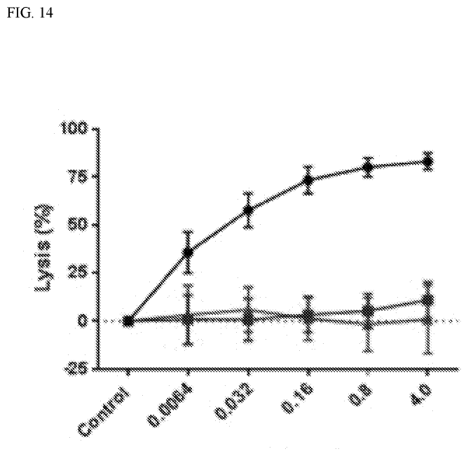

In newly diagnosed (ND) MM patient samples (n=8), the mean lysis of MM cells by JNJ-957 4.0 μg/mL was 79% (range: 66-92%; FIG. 12). Similar MM lysis, but with a larger variation, was achieved in lenalidomide (LEN) refractory patient samples (n=15; mean lysis at 4.0 μg/mL: 69%; range: 24-98%; FIG. 13), who were also bortezomib (73%), pomalidomide (82%) and carfilzomib (9%) refractory. JNJ-957 was also effective in samples from MM patients who were daratumumab (DARA) refractory (n=11; mean lysis at 4.0 μg/mL: 83%; range: 52-99%; FIG. 14). NK- and T-cell frequencies were not affected in any of the samples tested.

The CD3xnull and BCMAxnull control antibodies showed significantly lower activity in the different patient samples, when compared to JNJ-957, indicating the requirement for cross-linking of the MM cell and the effector T-cells, as well as absence of a direct effect of BCMA blockade.

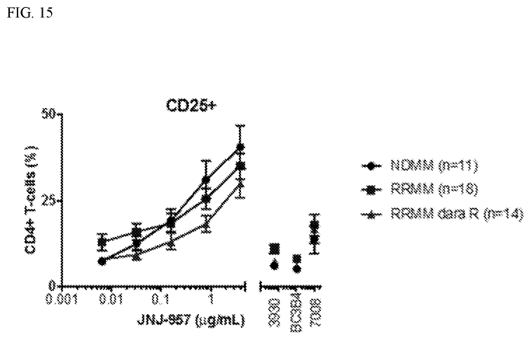

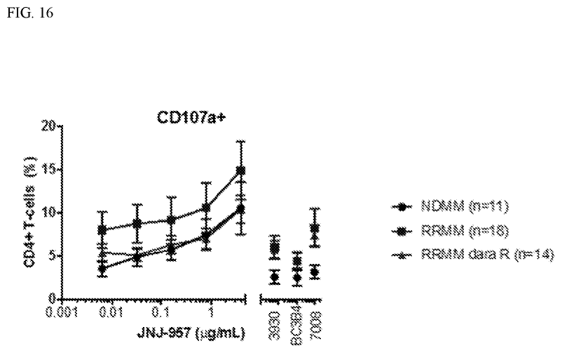

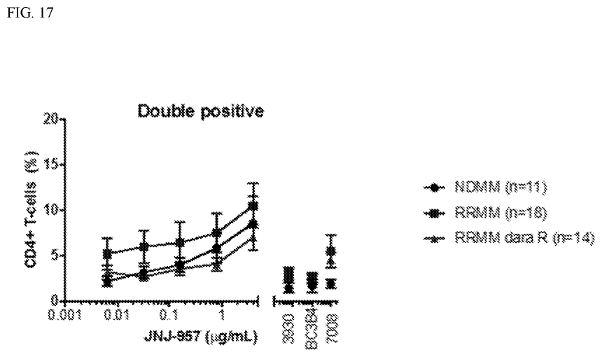

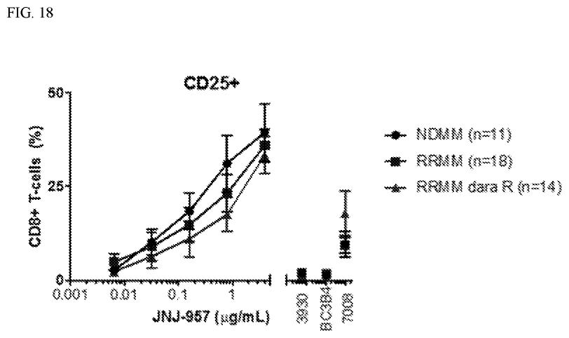

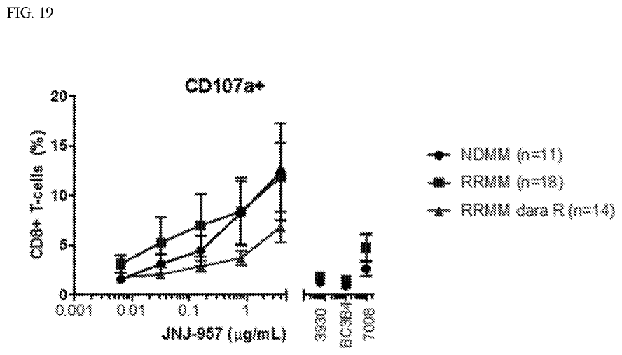

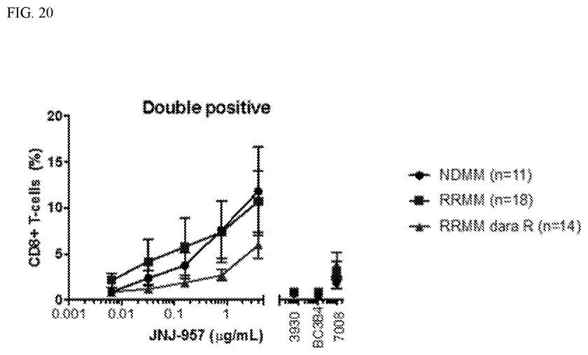

JNJ-957 mediated lysis of primary MM cells was associated with a dose-dependent increase in the percentage of activated CD4⁺ and CD8⁺ T-cells, as assessed by the expression of CD25 activation antigen. JNJ-957 treatment also resulted in degranulation of CD4⁺ and CD8⁺ T-cells, as determined by cell surface expression of CD107a. There was no difference in extent of T-cell activation and degranulation between NDMM, daratumumab-naïve RRMM and daratumumab-refractory RRMM patients. FIG. 15 shows JNJ-957-mediated increase in the percentage of CD25+ CD4 T cells. FIG. 16 shows JNJ-957-mediated increase in the percentage of CD107a+ CD4 T cells. FIG. 17 shows JNJ-957-mediated increase in the percentage of the double positive CD25+ CD107+CD4 T cells. FIG. 18 shows JNJ-957-mediated increase in the percentage of CD25+ CD8 T cells. FIG. 19 shows JNJ-957-mediated increase in the percentage of CD107a+ CD8 T cells. FIG. 20 shows JNJ-957-mediated increase in the percentage of the double positive CD25+ CD107+CD8 T cells.

Levels of granzyme B and various cytokines in the supernatant of the JNJ-957-treated BM-MNCs from daratumumab-naïve and daratumumab-refractory RRMM patients was also assessed. JNJ-957-mediated T-cell activation resulted in a dose-dependent increase in levels of granzyme B, IFN-γ, IL-2, IL-6, IL-8, IL-10, and TNF-α (data not shown).

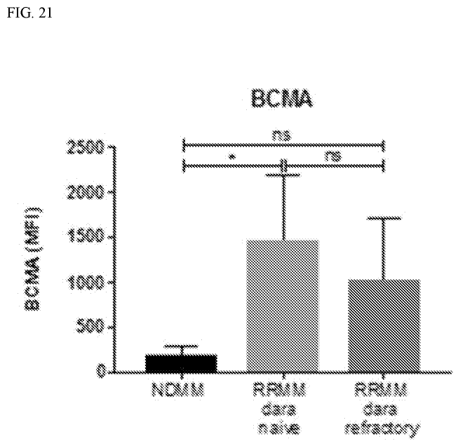

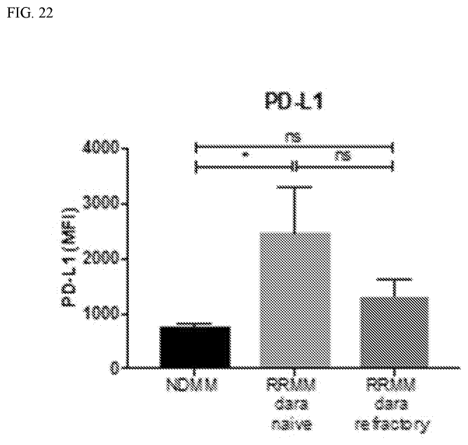

JNJ-957 efficacy in mediating MM cell killing was neither associated with tumor characteristics (BCMA or PD-L1 expression, the presence of standard or high-risk cytogenetic abnormalities) nor patient's characteristics such as effector:target ratio, composition of T-cell system or PD-1/HLA-DR expression on T-cells across all BM samples. However, when patient categories were analyzed separately, BCMA (FIG. 21) and PD-L1 (FIG. 22) expression levels were significantly higher in RRMM patients, compared to NDMM patients, irrespective of daratumumab exposure. Although patient numbers were small, the activity of JNJ-957 was inversely correlated with PD-L1 expression levels in daratumumab-naïve RRMM patients (P0.045).

The composition of the immune cells in the BM aspirates NDMM, daratumumab naïve RRMM and daratumumab RRMM samples were evaluated to gain understanding on the differential effect of JNJ-957 in samples obtained from the three patient subgroups. In the combined group of patients, a high T-cell frequency (P=0.034) and high E:T ratio (P=0.029) were associated with enhanced JNJ-7957-mediated lysis of MM cells. Other immune parameters (number of T-cells, Tregs, PD-1⁺ Tcells, HLA-DR⁻ T cells or naïve T cells) did not affect JNJ-7957 mediated MM cell lysis.

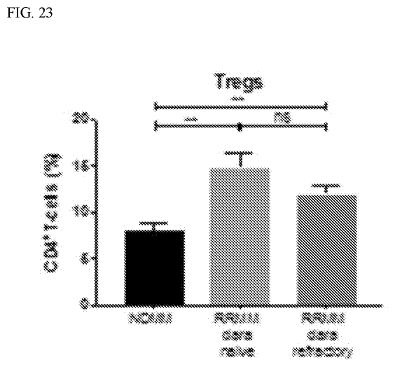

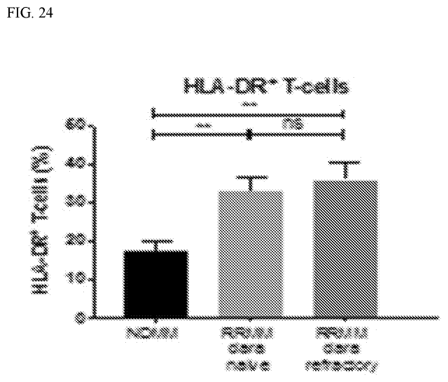

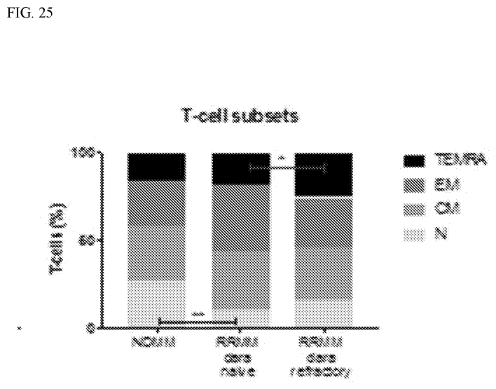

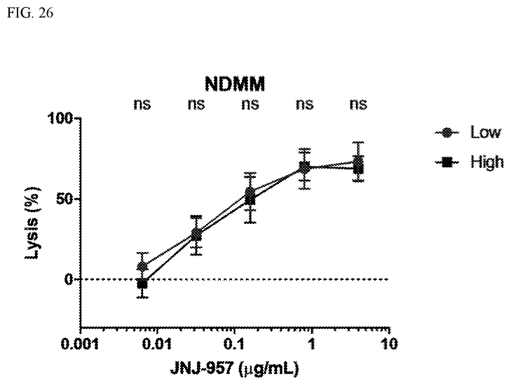

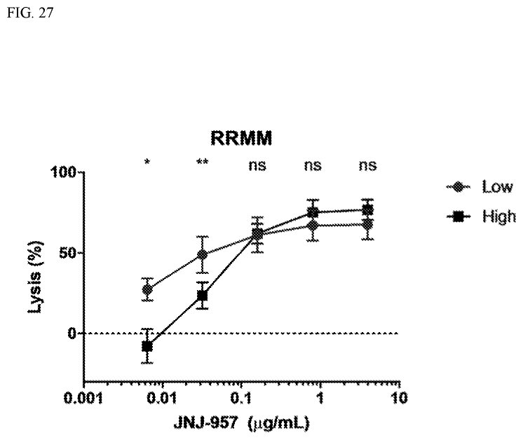

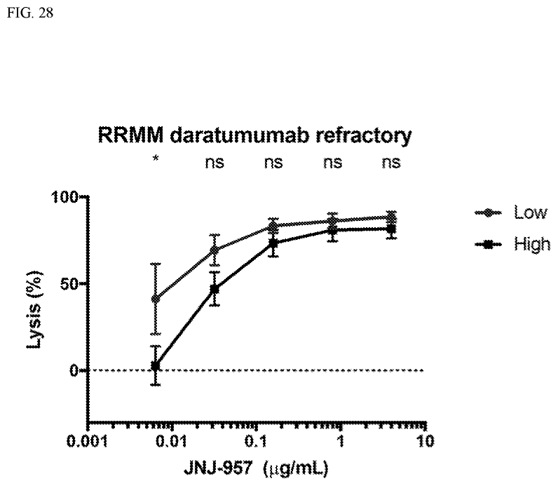

In the subgroup analysis, RRMM patients had a significantly higher frequency of Tregs (FIG. 23) and activated T-cells (defined by expression of HLA-DR) (FIG. 24), and a lower frequency of naïve T-cells, when compared to NDMM patients. In addition, daratumumab-refractory patient samples contained significantly more TEMRA T-cells than daratumumab-naïve samples (FIG. 25). However, frequencies of activated, naïve, central memory (CM), effector memory (EM) or TEMRA T-cells were not associated with response to JNJ-7957 in this subgroup analysis. A high baseline percentage of Tregs showed a negative influence on JNJ-957 mediated MM cell lysis in RRMM patient samples, which was overcome by optimal dosing. JNJ-597-mediated lysis of NDMM (FIG. 26), daratumumab naïve RRMM (FIG. 27) and daratumumab refractory RRMM (FIG. 28) patient samples mediated by autologous effector cells, dichotomized according to baseline percentage of Tregs was assessed. The 50^{th} percentile was used to categorize samples as “low” or “high” in terms of Treg content: NDMM: low: ≤7.34%, high:>7.34%. Daratumumab naïve RRMM: low≤15.57%, high >15.57%. Daratumumab refractory RRMM: low ≤11.24%, high >11.24%. Higher Treg concentration dampened JNJ-957-mediated lysis of MM cells in daratumumab naïve RRMM and daratumumab refractory RRMM samples. The Treg effect was abrogated at higher JNJ-957 concentrations.

The proportion of PD-1⁺ T-cells and E:T ratio were similar in the three patient groups. Only in NDMM patients, a low frequency of T-cells (P=0.010) and a high frequency of PD-1⁺ T-cells (P=0.048) impaired JNJ-957-mediated lysis of MM cells (data not shown).

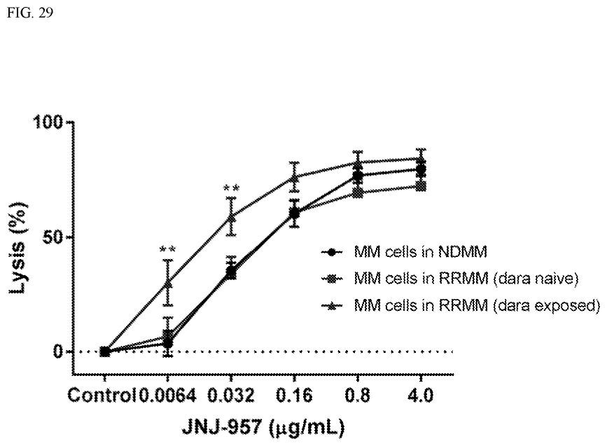

The effect of daratumumab treatment to JNJ-957 efficacy was evaluated by assessing JNJ-957-mediated lysis in BM samples from NDMM (n=9), daratumumab naïve RRMM (n=18) and daratumumab-refractory RRMM (n=13) patients after a 48-hour incubation. At relatively low concentrations of JNJ-957 (0.0064-0.032 μg/mL), tumor cell lysis was significantly better in the daratumumab-exposed patients, as compared to both daratumumab naïve RRMM and NDMM patients. FIG. 29 shows the percentage lysis in the patient populations. Data are depicted as mean±SEM, P values are calculated using student t-test.

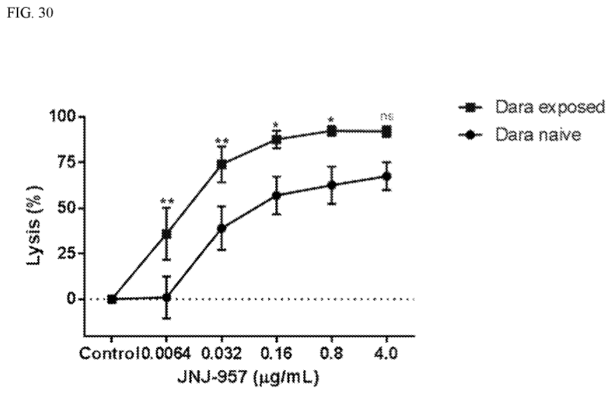

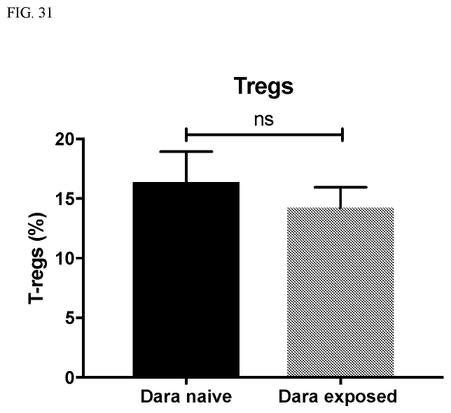

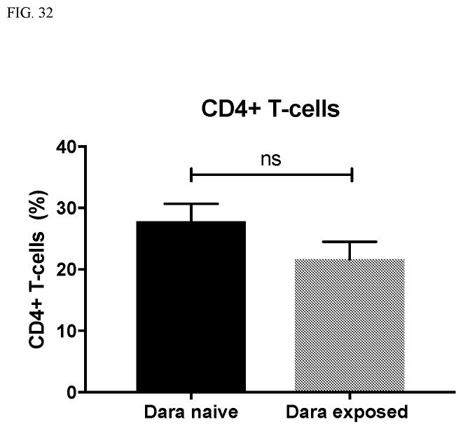

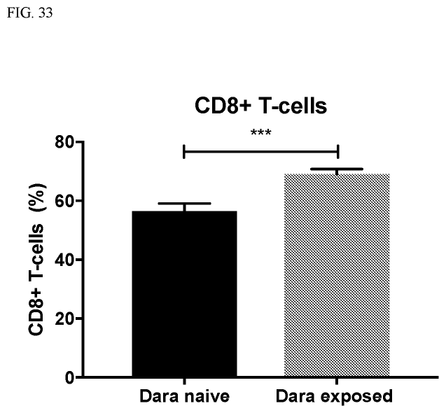

Since improvement in tumor reduction could be aided by the recently discovered immune stimulatory effects of DARA, sequential BM aspirates from MM patients were analyzed before and after DARA treatment (n=5). Here we observed comparable BCMA expression, yet improved MM cell lysis by JNJ-957 in samples obtained after disease progression during DARA compared to samples before DARA initiation (mean lysis at 4.0 μg/mL: 93 vs 74%; FIG. 30). In these BM aspirates, the percentage of Tregs (FIG. 31) and CD4⁺ cells (FIG. 32) were slightly decreased whereas the percentage of CD8+ cells (FIG. 33) was increased in daratumumab naïve vs. daratumumab exposed patient samples. In this study, the samples were obtained from patients whose median duration of daratumumab monotherapy treatment of patients was 3 (1-7) months. In a follow-up study with samples from 8 RRMM patients, the percentage of CD38⁺ Tregs and Bregs were significantly reduced in dara refractory vs. daratumumab naïve patient samples (data not shown).

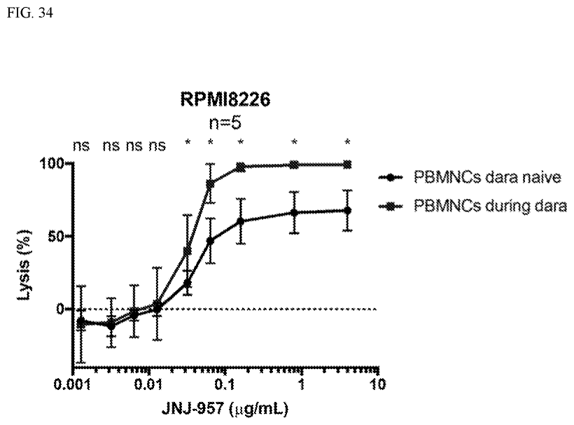

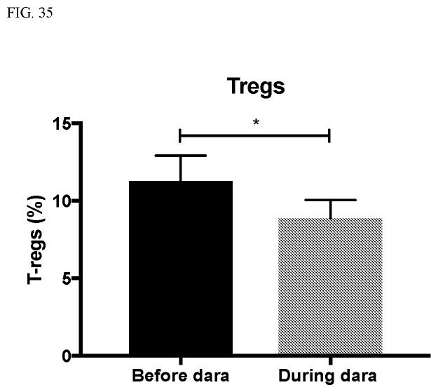

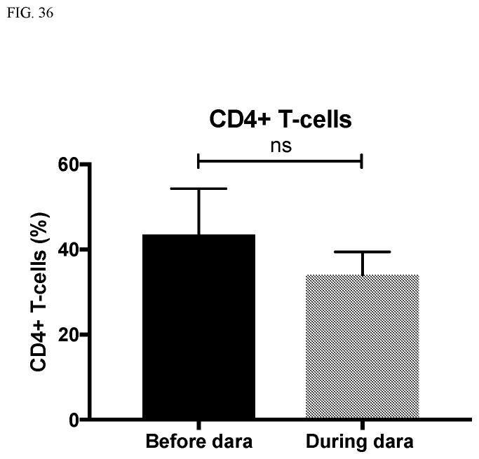

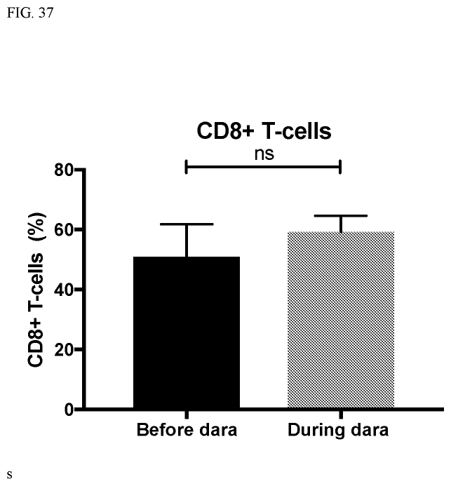

JNJ-957-mediated lysis of RPMI 8226 multiple myeloma cell line was tested using sequential PB MNC samples from RRMM patients before and during daratumumab treatment as effector cells. Dara exposed PB MNCs were obtained during daratumumab treatment from patients with good response (either partial response, very good partial response or complete response) with median duration of daratumumab treatment 11 months (range 7-14 months). FIG. 34 shows that JNJ-957 mediated lysis of RPMI 8226 was enhanced using PB MNCs from dara exposed patients. In the PB-MNC samples, the percentage of Tregs (FIG. 35) and CD4+ cells (FIG. 36) were slightly decreased whereas the percentage of CD8+ cells (FIG. 37) was increased in daratumumab naïve vs. daratumumab exposed patient samples. In this study, the samples were obtained from patients whose median duration of daratumumab treatment of patients was 3 (1-7) months.

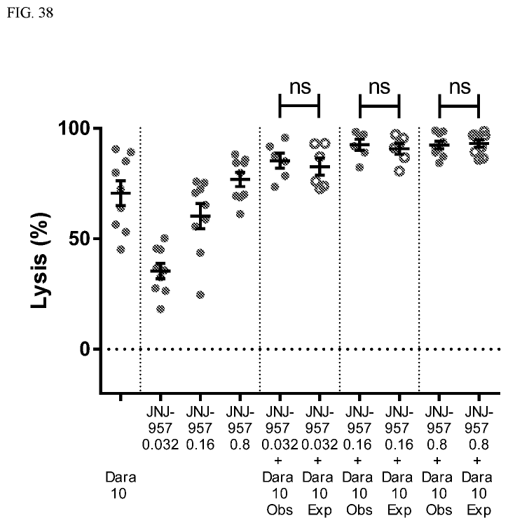

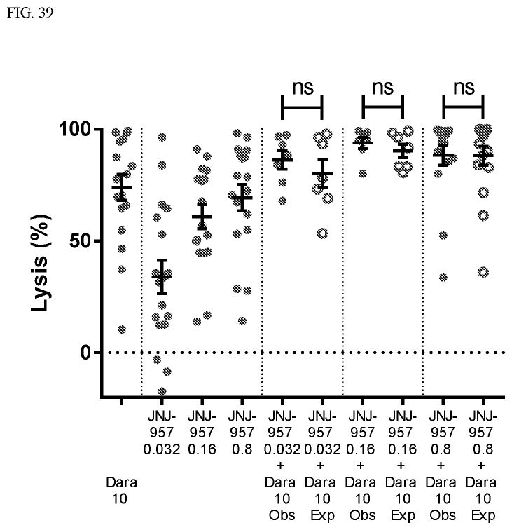

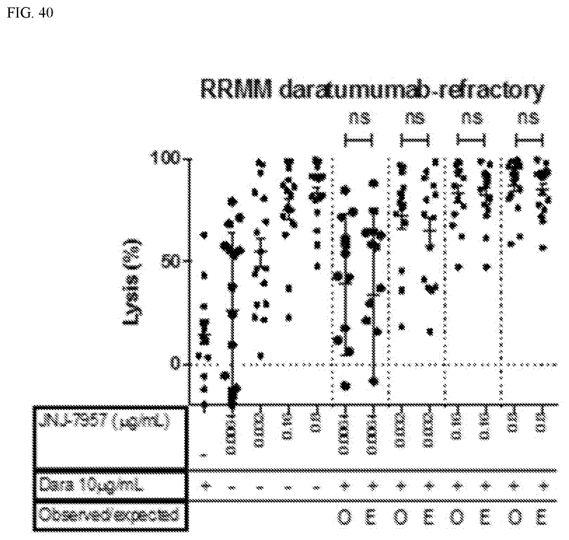

Combination of JNJ-957 and daratumumab was also tested for the efficacy in killing MM cells obtained from NDMM or RRMM dara naïve patients. FIG. 38 shows the percentage lysis of BM MNC of newly diagnosed MM (NDMM) (n=8) patients treated with JNJ-957 (0.032-0.8 μg/mL) alone or in combination with daratumumab 10 μg/mL for 48 hours. The observed (obs) lysis levels of MM cells by JNJ-957 and daratumumab were compared to the expected (exp) lysis levels, which were calculated with the assumption that the combinatorial effect is achieved by additive effects as indicated in methods. Black bars depict the group mean value±SEM. P values are calculated using a paired student t-test. FIG. 39 shows the percentage lysis of BM MNCs inf RRNN dara naïve patients. FIG. 40 shows the percentage lysis of BM MNCs in RRMM daratumumab refractory patients.

The study therefore demonstrated that JNJ-957 was effective in newly diagnosed and heavily pretreated MM patient samples. A high percentage or regulatory T cells negatively influenced JNJ-957 efficacy at low dosages however the negative effect was overcome by dose increase of JNJ-957. Daratumumab pretreatment in vivo enhanced the efficacy of JNJ-957 against MM cells.

The combination of JNJ-957 and daratumumab ex vivo showed additive efficacy; furthermore, in vivo pretreatment with daratumumab augmented the ex vivo efficacy of BCMAxCD3.

### Example 3 Daratumumab Treatment Enhanced Ex Vivo Efficacy of Blinatumomab

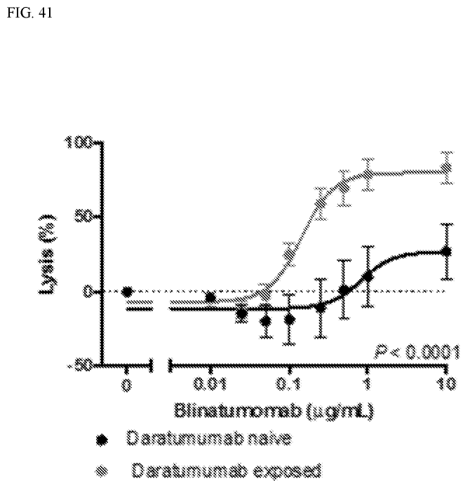

To assess if daratumumab treatment is also beneficial for other T-cell redirecting therapies, CD19⁺ Raji cells were treated with blinatumomab, an FDA-approved CD19xCD3 BiTE for the treatment of acute lymphoblastic leukemia, using paired daratumumab-naïve and daratumumab exposed PB-MNCs from 11 MM patients. Similar to the observations with JNJ-957, the activity of blinatumomab was significantly enhanced by co-incubation with daratumumab-exposed PB-MNCs, when compared to daratumumab-naïve PB-MNCs (P<0.0001; FIG. 41). Blinatumomab comprises the amino acid sequence of SEQ ID NO: 53.

SEQ ID NO: 53
DIQLTQSPASLAVSLGQRATISCKASQSVDYDGDSYLNWYQQIPGQPPKL
LIYDASNLVSGIPPRFSGSGSGTDFTLNIHPVEKVDAATYHCQQSTEDPW
TFGGGTKLEIKGGGGSGGGGSGGGGSQVQLQQSGAELVRPGSSVKISCKA
SGYAFSSYWMNWVKQRPGQGLEWIGQIWPGDGDTNYNGKFKGKATLTADE
SSSTAYMQLSSLASEDSAVYFCARRETTTVGRYYYAMDYWGQGTTVTVSS
GGGGSDIKLQQSGAELARPGASVKMSCKTSGYTFTRYTMHWVKQRPGQGL
EWIGYINPSRGYTNYNQKFKDKATLTTDKSSSTAYMQLSSLTSEDSAVYY
CARYYDDHYCLDYWGQGTTLTVSSVEGGSGGSGGSGGSGGVDDIQLTQSP
AIMSASPGEKVTMTCRASSSVSYMNWYQQKSGTSPKRWIYDTSKVASGVP
YRFSGSGSGTSYSLTISSMEAEDAATYYCQQWSSNPLTFGAGTKLELKHH
HHHH

### Example 4 JNJ-957 Effectively Killed Primary pPCL Cells

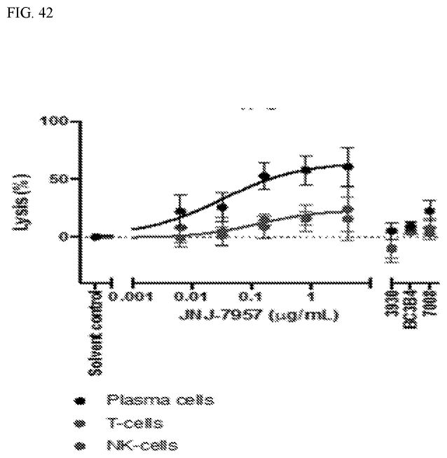

Ex vivo activity of JNJ-957 was evaluated in BM samples from 6 patients with newly diagnosed pPCL, which is characterized by an aggressive clinical behavior. JNJ-957 mediated tumor cell lysis in these pPCL samples was similar to lysis observed in NDMM and daratumumab-naïve RRMM samples, but lower than observed in daratumumab-refractory RRMM patient samples (P=0.0014) (FIG. 42). Although the median E:T ratio in pPCL samples was approximately 8-fold lower, the extent of activation of both CD4⁺ (P=0.0040) and CD8⁺ T-cells (P<0.0001), as well as the extent of degranulation of CD8⁺ T-cells (P=0.0141) was superior in pPCL, when compared to NDMM. Degranulation of CD4⁺ T-cells was similar to that observed in NDMM.

BM-MNCs were obtained from 6 pPCL patients and incubated with JNJ-957 (0.0064-4.0 μg/mL) or control antibodies 3930, BC3B4 and 7008 (4.0 μg/mL) for 48 hours, after which the surviving CD138⁺ tumor cells, as well as T- and NK-cells, were enumerated using flow cytometry analysis. Data was expressed as mean % lysis of cells ±SEM. All experiments were performed in duplicate.

### Example 5 Combination of a GPRC5DxCD3 Bispecific Antibody with Daratumumab

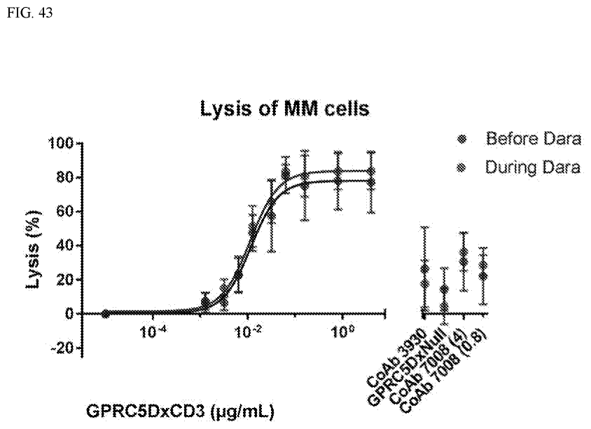

To further assess if daratumumab treatment is also beneficial for other T-cell redirecting therapies, RPMI MM cells were treated with a GPRC5DxCD3 bispecific antibody using paired daratumumab-naïve and daratumumab exposed PB-MNCs from 11 MM patients (the samples were obtained from the same patients as described in above examples. As a control, antibodies in which either the CD3 or the GPRC5D binding VH/VL domains were replaced with null domains binding irrelevant antigens (gp120) were used (control mAb 3930 nullxnull, control mAb 7008: NullxCD3, control mAb GPRC5Dxnull). The antibodies were tested over a concentration of 0.00064-4.0 μg/ml. The GPRC5DxCD3 bispecific antibody mediated MM cell lysis in both daratumumab naïve and daratumumab refractory samples with similar potency (FIG. 43).

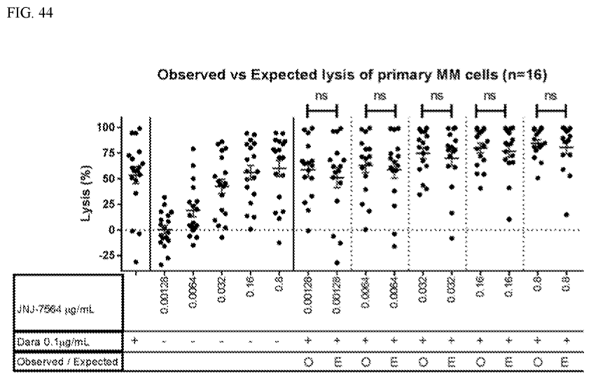

Combination of the GPRC5DxCD3 bispecific antibody and daratumumab was also tested for the efficacy in killing MM cells obtained from NDMM or RRMM dara naïve patients. FIG. 44 shows the percentage lysis of BM MNC of primary MM cells mediated by the GPRC5DxCD3 bispecific antibody (0.0128-0.8 μg/mL) alone or in combination with daratumumab 0.1 μg/mL for 48 hours. The observed (0) lysis levels of MM cells by the GPRC5DxCD3 bispecific antibody and daratumumab were compared to the expected (E) lysis levels, which were calculated with the assumption that the combinatorial effect is achieved by additive effects as indicated in methods. Black bars depict the group mean value±SEM. P values were calculated using a paired student t-test. Co-incubation with daratumumab enhanced MM cell lysis by the GPRC5DxCD3 bispecific antibody in an additive fashion.

The GPRC5DxCD3 bispecific antibody comprises a GPRC5D binding arm GC5B596 and a CD3 binding arm CD3B219. The amino acid sequences of GC5B596 are shown in Table 8. The amino acid sequences of CD3B219 are show in Table 4.

The GPRC5DxCD3 bispecific antibody used in the experiments is described in WO20180037651A1 and comprises the following sequences:
a GPRC5D binding domain comprising the HCDR1, the HCDR2, the HCDR3, the LCDR1, the LCDR2 and the LCDR3 of SEQ ID NOs: 43, 44, 45, 446, 47 and 48, respectively, and a CD3 binding domain comprising the HCDR1, the HCDR2, the HCDR3, the LCDR1, the LCDR2 and the LCDR3 of SEQ ID NOs: 33, 34, 35, 36, 37 and 38, respectively;
the GPRC5D binding domain comprising the VH of SEQ ID NO: 49 and the VL of SEQ ID NO: 50 and the CD3 binding domain comprises the VH of SEQ ID NO: 39 and the VL of SEQ ID NO: 40; and
a first heavy chain (HC1) of SEQ ID NO: 51, a first light chain (LC1) of SEQ ID NO: 52, a second heavy chain (HC2) of SEQ ID NO: 41 and a second light chain (LC2) of SEQ ID NO: 42.

The GPRC5DxCD3 bispecific antibody is an IgG4 isotype.

The HC1 comprises 228P, F234A and L235A substitutions.

The HC2 comprises 228P, F234A, L235A, F405L and R409K substitutions.

TABLE 8

| PS3B27 | Region | Sequence | SEQ ID NO: |
|---|---|---|---|
| GC5B596 | HCDR1 | GYTMN | 43 |
| | HCDR2 | LINPYNSDTNYAQKLQG | 44 |
| | HCDR3 | VALRVALDY | 45 |
| | LCDR1 | KASQNVATHVG | 46 |
| | LCDR2 | SASYRYS | 47 |
| | LCDR3 | QQYNRYPYT | 48 |
| | VH | QVQLVQSGAEVKKPGASVKVSCKASGYSFTGYT | 49 |
| | | MNWVRQAPGQGLEWMGLINPYNSDTNYAQKL | |
| | | QGRVTMTTDTSTSTAYMELRSLRSDDTAVYYCA | |
| | | RVALRVALDYWGQGTLVTVSS | |
| | VL | DIQMTQSPSSLSASVGDRVTITCKASQNVATHVG | 50 |
| | | WYQQKPGKAPKRLIYSASYRYSGVPSRFSGSGSG | |
| | | TEFTLTISNLQPEDFATYYCQQYNRYPYTFGQGT | |
| | | KLEIK | |
| | HC | QVQLVQSGAEVKKPGASVKVSCKASGYSFTGYT | 51 |
| | | MNWVRQAPGQGLEWMGLINPYNSDTNYAQKL | |
| | | QGRVTMTTDTSTSTAYMELRSLRSDDTAVYYCA | |
| | | RVALRVALDYWGQGTLVTVSS | |
| | | ASTKGPSVFPLAPCSRSTSESTAALGCLVKDYFPE | |
| | | PVTVSWNSGALTSGVHTFPAVLQSSGLYSLSSVV | |
| | | TVPSSSLGTKTYTCNVDHKPSNTKVDKRVESKY | |
| | | GPPCPPCPAPEAAGGPSVFLFPPKPKDTLMISRTP | |
| | | EVTCVVVDVSQEDPEVQFNWYVDGVEVHNAKT | |
| | | KPREEQFNSTYRVVSVLTVLHQDWLNGKEYKCK | |
| | | VSNKGLPSSIEKTISKAKGQPREPQVYTLPPSQEE | |
| | | MTKNQVSLTCLVKGFYPSDIAVEWESNGQPENN | |
| | | YKTTPPVLDSDGSFFLYSRLTVDKSRWQEGNVFS | |
| | | CSVMHEALHNHYTQKSLSLSLGK | |
| | LC | DIQMTQSPSSLSASVGDRVTITCKASQNVATHVG | 52 |
| | | WYQQKPGKAPKRLIYSASYRYSGVPSRFSGSGSG | |
| | | TEFTLTISNLQPEDFATYYCQQYNRYPYTFGQGT | |
| | | KLEIKKAAPSVTLFPPSSEELQANKATLVCLISDF | |
| | | YPGAVTVAWKGDSSPVKAGVETTTPSKQSNNKY | |
| | | AASSYLSLTPEQWKSHRSYSCQVTHEGSTVEKTV | |
| | | APTECS | |

### Example 6 Combinations of T-Cell Redirecting Therapies with Anti-CD38 Antibodies

Effect of combining additional T-cell redirecting therapies with anti-CD38 antibodies is assessed similarly as described in Examples 1-5. The combinations are tested for their additive or synergistic effect to mediate killing of tumor cells that are targeted by the T-cell redirecting therapies (i.e., tumor cells that express the antigen that is bound by the T-cell redirecting therapy). The effect of pre-treatment of anti-CD38 antibodies on efficacy of T-cell redirecting therapies is assessed as described herein in the Examples.

The T-cell redirecting therapies that are tested in combination with anti-CD38 antibodies include PSMAxCD3, TMEFF2xCD3, CD123xCD3 and CD33xCD3 bispecific antibodies.

An exemplary PSMAxCD3 bispecific antibody is PS3B27, comprising a PSMA binding domain PSMB127 and the CD3 binding domain CD3B219. Table 9 shows the amino acid sequences of PS3B27. The amino acid sequences of CD3B219 are show in Table 4.

An exemplary PSMAxCD3 bispecific antibody that is used in the experiments comprises the following sequences:
a PSMA binding domain comprising the HCDR1, the HCDR2, the HCDR3, the LCDR1, the LCDR2 and the LCDR3 of SEQ ID NOs: 54, 55, 56, 9, 10 and 59, respectively, and a CD3 binding domain comprising the HCDR1, the HCDR2, the HCDR3, the LCDR1, the LCDR2 and the LCDR3 of SEQ ID NOs: 33, 34, 35, 36, 37 and 38, respectively;
the PSMA binding domain comprising the VH of SEQ ID NO: 60 and the VL of SEQ ID NO: 61 and the CD3 binding domain comprises the VH of SEQ ID NO: 39 and the VL of SEQ ID NO: 40; and
a first heavy chain (HC1) of SEQ ID NO: 62, a first light chain (LC1) of SEQ ID NO: 63, a second heavy chain (HC2) of SEQ ID NO: 41 and a second light chain (LC2) of SEQ ID NO: 42.

The anti-PSMAxCD3 bispecific antibody is an IgG4 isotype.

The HC1 comprises S228P, F234A and L235A substitutions.

The HC2 comprises S228P, F234A, L235A, F405L and R409K substitutions.

TABLE 9

| | Region | Sequence | SEQ ID NO: |
|---|---|---|---|
| PSMB127 | HCDR1 | SDAMH | 54 |
| | HCDR2 | EISGSGGYTNYADSVKG | 55 |
| | HCDR3 | DSYDSSLYVGDYFDY | 56 |
| | LCDR1 | RASQSVSSYLA | 9 |
| | LCDR2 | DASNRAT | 10 |
| | LCDR3 | QQRSNWPLT | 59 |
| | VH | EVQLLESGGGLVQPGGSLRLSCAASGFTFKSDA | 60 |
| | | MHWVRQAPGKGLEWVSEISGSGGYTNYADSVK | |
| | | GRFTISRDNSKNTLYLQMNSLRAEDTAVYYCAR | |
| | | DSYDSSLYVGDYFDYWGQGTLVTVSS | |
| | VL | EIVLTQSPATLSLSPGERATLSCRASQSVSSYLAW | 61 |
| | | YQQKPGQAPRLLIYDASNRATGIPARFSGSGSGT | |
| | | DFTLTISSLEPEDFAVYYCQQRSNWPLTFGQGTK | |
| | | VEIK | |
| | HC | EVQLLESGGGLVQPGGSLRLSCAASGFTFKSDA | 62 |
| | | MHWVRQAPGKGLEWVSEISGSGGYTNYADSVK | |
| | | GRFTISRDNSKNTLYLQMNSLRAEDTAVYYCAR | |
| | | DSYDSSLYVGDYFDYWGQGTLVTVSSASTKGPS | |
| | | VFPLAPCSRSTSESTAALGCLVKDYFPEPVTVSW | |
| | | NSGALTSGVHTFPAVLQSSGLYSLSSVVTVPSSSL | |
| | | GTKTYTCNVDHKPSNTKVDKRVESKYGPPCPPC | |
| | | PAPEAAGGPSVFLFPPKPKDTLMISRTPEVTCVV | |
| | | VDVSQEDPEVQFNWYVDGVEVHNAKTKPREEQ | |
| | | FNSTYRVVSVLTVLHQDWLNGKEYKCKVSNKG | |
| | | LPSSIEKTISKAKGQPREPQVYTLPPSQEEMTKNQ | |
| | | VSLTCLVKGFYPSDIAVEWESNGQPENNYKTTPP | |
| | | VLDSDGSFFLYSRLTVDKSRWQEGNVFSCSVMH | |
| | | EALHNHYTQKSLSLSLGK | |
| | LC | EIVLTQSPATLSLSPGERATLSCRASQSVSSYLAW | 63 |
| | | YQQKPGQAPRLLIYDASNRATGIPARFSGSGSGT | |
| | | DFTLTISSLEPEDFAVYYCQQRSNWPLTFGQGTK | |
| | | VEIKRTVAAPSVFIFPPSDEQLKSGTASVVCLLNN | |
| | | FYPREAKVQWKVDNALQSGNSQESVTEQDSKDS | |
| | | TYSLSSTLTLSKADYEKHKVYACEVTHQGLSSPV | |
| | | TKSFNRGEC | |

An exemplary TMEFF2xCD3 bispecific antibody is TMCB150, comprising a TMEFF2 binding arm TMEB762 and the CD3 binding arm CD3B376. Table 10 shows the amino acid sequences of TMEB762. Table 11 shows the amino acid sequences of CD3B376.

An exemplary TMEFF2xCD3 bispecific antibody that is used in the experiments is TMCB150 and comprises the following sequences:
a TMEFF2 binding domain comprising the HCDR1, the HCDR2, the HCDR3, the LCDR1, the LCDR2 and the LCDR3 of SEQ ID NOs: 64, 65, 66, 67, 68 and 69, respectively, and a CD3 binding domain comprising the HCDR1, the HCDR2, the HCDR3, the LCDR1, the LCDR2 and the LCDR3 of SEQ ID NOs: 74, 75, 76, 77, 78 and 79, respectively;
the TMEFF2 binding domain comprising the VH of SEQ ID NO: 70 and the VL of SEQ ID NO: 71 and the CD3 binding domain comprises the VH of SEQ ID NO: 80 and the VL of SEQ ID NO: 81; and
a first heavy chain (HC1) of SEQ ID NO: 72, a first light chain (LC1) of SEQ ID NO: 73, a second heavy chain (HC2) of SEQ ID NO: 82 and a second light chain (LC2) of SEQ ID NO: 83.

The anti-TMEFF2xCD3 bispecific antibody is an IgG4 isotype.

The HC1 comprises S228P, F234A and L235A substitutions.

The HC2 comprises S228P, F234A, L235A, F405L and R409K substitutions.

TABLE 10

| | Region | Sequence | SEQ ID NO: |
|---|---|---|---|
| TMEB762 | HCDR1 | SYSMS | 64 |
| | HCDR2 | VISGSGGFTDYADSVKG | 65 |
| | HCDR3 | MPLNSPHDY | 66 |
| | LCDR1 | RASQGIRNDLG | 67 |
| | LCDR2 | AASSLQS | 68 |
| | LCDR3 | LQDYNYPLT | 69 |
| | VH | EVQLLESGGGLVQPGGSLRLSCAASGFTFSSYSM | 70 |
| | | SWVRQAPGKGLEWVSVISGSGGFTDYADSVKGR | |
| | | FTISRDNSKNTLYLQMNSLRAEDTAVYYCARMP | |
| | | LNSPHDYWGQGTLVTVSS | |
| | VL | DIQMTQSPSSLSASVGDRVTITCRASQGIRNDLG | 71 |
| | | WYQQKPGKAPKLLIYAASSLQSGVPSRFSGSGSG | |
| | | TDFTLTISSLQPEDFATYYCLQDYNYPLTFGGGT | |
| | | KVEIK | |
| | HC | VQLLESGGGLVQPGGSLRLSCAASGFTFSSYSMS | 72 |
| | | WVRQAPGKGLEWVSVISGSGGFTDYADSVKGRF | |
| | | TISRDNSKNTLYLQMNSLRAEDTAVYYCARMPL | |
| | | NSPHDYWGQGTLVTVSSASTKGPSVFPLAPCSRS | |
| | | TSESTAALGCLVKDYFPEPVTVSWNSGALTSGV | |
| | | HTFPAVLQSSGLYSLSSVVTVPSSSLGTKTYTCN | |
| | | VDHKPSNTKVDKRVESKYGPPCPPCPAPEAAGG | |
| | | PSVFLFPPKPKDTLMISRTPEVTCVVVDVSQEDPE | |
| | | VQFNWYVDGVEVHNAKTKPREEQFNSTYRVVS | |
| | | VLTVLHQDWLNGKEYKCKVSNKGLPSSIEKTISK | |
| | | AKGQPREPQVYTLPPSQEEMTKNQVSLTCLVKG | |
| | | FYPSDIAVEWESNGQPENNYKTTPPVLDSDGSFF | |
| | | LYSRLTVDKSRWQEGNVFSCSVMHEALHNHYT | |
| | | QKSLSLSLGK | |
| | LC | DIQMTQSPSSLSASVGDRVTITCRASQGIRNDLG | 73 |
| | | WYQQKPGKAPKLLIYAASSLQSGVPSRFSGSGSG | |
| | | TDFTLTISSLQPEDFATYYCLQDYNYPLTFGGGT | |
| | | KVEIKRTVAAPSVFIFPPSDEQLKSGTASVVCLLN | |
| | | NFYPREAKVQWKVDNALQSGNSQESVTEQDSK | |
| | | DSTYSLSSTLTLSKADYEKHKVYACEVTHQGLSS | |
| | | PVTKSFNRGEC | |

TABLE 11

| | Region | Sequence | SEQ ID NO: |
|---|---|---|---|
| CD3B396 | HCDR1 | NNNAAWS | 74 |
| | HCDR2 | RTYYRSKWLYDYAVSVKS | 75 |
| | HCDR3 | GYSSSFDY | 76 |
| | LCDR1 | TGTSSNIGTYKFVS | 77 |
| | LCDR2 | EVSKRPS | 78 |
| | LCDR3 | VSYAGSGTLL | 79 |
| | VH | QVQLQQSGPRLVRPSQTLSLTCAISGDSVFNNNA | 80 |
| | | AWSWIRQSPSRGLEWLGRTYYRSKWLYDYAVS | |
| | | VKSRITVNPDTSRNQFTLQLNSVTPEDTALYYCA | |
| | | RGYSSSFDYWGQGTLVTVSS | |
| | VL | QSALTQPASVSGSPGQSITISCTGTSSNIGTYKFVS | 81 |
| | | WYQQHPDKAPKVLLYEVSKRPSGVSSRFSGSKS | |
| | | GNTASLTISGLQAEDQADYHCVSYAGSGTLLFG | |
| | | GGTKLTVL | |
| | HC | QVQLQQSGPRLVRPSQTLSLTCAISGDSVFNNNA | 82 |
| | | AWSWIRQSPSRGLEWLGRTYYRSKWLYDYAVS | |
| | | VKSRITVNPDTSRNQFTLQLNSVTPEDTALYYCA | |
| | | RGYSSSFDYWGQGTLVTVSSASTKGPSVFPLAPC | |
| | | SRSTSESTAALGCLVKDYFPEPVTVSWNSGALTS | |
| | | GVHTFPAVLQSSGLYSLSSVVTVPSSSLGTKTYT | |
| | | CNVDHKPSNTKVDKRVESKYGPPCPPCPAPEAA | |
| | | GGPSVFLFPPKPKDTLMISRTPEVTCVVVDVSQE | |
| | | DPEVQFNWYVDGVEVHNAKTKPREEQFNSTYR | |
| | | VVSVLTVLHQDWLNGKEYKCKVSNKGLPSSIEK | |
| | | TISKAKGQPREPQVYTLPPSQEEMTKNQVSLTCL | |
| | | VKGFYPSDIAVEWESNGQPENNYKTTPPVLDSD | |
| | | GSFLLYSKLTVDKSRWQEGNVFSCSVMHEALHN | |
| | | HYTQKSLSLSLGK | |
| | LC | QSALTQPASVSGSPGQSITISCTGTSSNIGTYKFVS | 83 |
| | | WYQQHPDKAPKVLLYEVSKRPSGVSSRFSGSKS | |
| | | GNTASLTISGLQAEDQADYHCVSYAGSGTLLFG | |
| | | GGTKLTVLGQPKAAPSVTLFPPSSEELQANKATL | |
| | | VCLISDFYPGAVTVAWKADSSPVKAGVETTTPSK | |
| | | QSNNKYAASSYLSLTPEQWKSHRSYSCQVTHEG | |
| | | STVEKTVAPTECS | |

An exemplary CD33xCD3 bispecific antibody is C3CB189, comprising a CD33 binding arm C33B904 and the CD3 binding arm CD3B376. Table 12 shows the amino acid sequences of C33B904. The amino acid sequences of CD3B376 are shown in Table 11.

An exemplary CD33xCD3 bispecific antibody that is used in the experiments is C3CB189 and comprises the following sequences:
a CD33 binding domain comprising the HCDR1, the HCDR2, the HCDR3, the LCDR1, the LCDR2 and the LCDR3 of SEQ ID NOs: 84, 85, 86, 87, 88 and 89, respectively, and a CD3 binding domain comprising the HCDR1, the HCDR2, the HCDR3, the LCDR1, the LCDR2 and the LCDR3 of SEQ ID NOs: 74, 75, 76, 77, 78 and 79, respectively;
the CD33 binding domain comprising the VH of SEQ ID NO: 90 and the VL of SEQ ID NO: 91 and the CD3 binding domain comprises the VH of SEQ ID NO: 80 and the VL of SEQ ID NO: 81; and
a first heavy chain (HC1) of SEQ ID NO: 92, a first light chain (LC1) of SEQ ID NO: 93, a second heavy chain (HC2) of SEQ ID NO: 82 and a second light chain (LC2) of SEQ ID NO: 83.

The anti-CD33xCD3 bispecific antibody is an IgG4 isotype.

The HC1 comprises S228P, F234A and L235A substitutions.

The HC2 comprises S228P, F234A, L235A, F405L and R409K substitutions.

TABLE 12

| | Region | Sequence | SEQ ID NO: |
|---|---|---|---|
| C33B904 | HCDR1 | DYAMH | 84 |
| | HCDR2 | GIGWSGGSIVYADSVKG | 85 |
| | HCDR3 | DSPYGDFFDY | 86 |
| | LCDR1 | KSSQTVFYSSNNKNYLA | 87 |
| | LCDR2 | WASTRKS | 88 |
| | LCDR3 | QHYYSTPYT | 89 |
| | VH | EVQLVESGGGLVQPGRSLRLSCAASGFTFDDYA | 90 |
| | | MHWVRQAPGKGLEWVSGIGWSGGSIVYADSVK | |
| | | GRFTISRDNAKNSLYLQMNSLRAEDTALYYCAK | |
| | | DSPYGDFFDYWGQGTLVTVSS | |
| | VL | DIVMTQSPDSLAVSLGERATINCKSSQTVFYSSN | 91 |
| | | NKNYLAWYQQKPGQPPKLLISWASTRKSGVPD | |
| | | RFSGSGSGTDFTLTVSSLQAEDVAVYYCQHYY | |
| | | STPYTFGQGTKLEIK | |
| | HC | EVQLVESGGGLVQPGRSLRLSCAASGFTFDDYA | 92 |
| | | MHWVRQAPGKGLEWVSGIGWSGGSIVYADSVK | |
| | | GRFTISRDNAKNSLYLQMNSLRAEDTALYYCAK | |
| | | DSPYGDFFDYWGQGTLVTVSSASTKGPSVFPLAP | |
| | | CSRSTSESTAALGCLVKDYFPEPVTVSWNSGALT | |
| | | SGVHTFPAVLQSSGLYSLSSVVTVPSSSLGTKTYT | |
| | | CNVDHKPSNTKVDKRVESKYGPPCPPCPAPEAA | |
| | | GGPSVFLFPPKPKDTLMISRTPEVTCVVVDVSQE | |
| | | DPEVQFNWYVDGVEVHNAKTKPREEQFNSTYR | |
| | | VVSVLTVLHQDWLNGKEYKCKVSNKGLPSSIEK | |
| | | TISKAKGQPREPQVYTLPPSQEEMTKNQVSLTCL | |
| | | VKGFYPSDIAVEWESNGQPENNYKTTPPVLDSD | |
| | | GSFFLYSRLTVDKSRWQEGNVFSCSVMHEALHN | |
| | | HYTQKSLSLSLGK | |
| | LC | DIVMTQSPDSLAVSLGERATINCKSSQTVFYSSN | 93 |
| | | NKNYLAWYQQKPGQPPKLLISWASTRKSGVPDR | |
| | | FSGSGSGTDFTLTVSSLQAEDVAVYYCQHYYSTP | |
| | | YTFGQGTKLEIKKAAPSVTLFPPSSEELQANKATL | |
| | | VCLISDFYPGAVTVAWKGDSSPVKAGVETTTPSK | |
| | | QSNNKYAASSYLSLTPEQWKSHRSYSCQVTHEG | |
| | | STVEKTVAPTECS | |

An exemplary CD123xCD3 bispecific antibody is 8747, comprising a CD123 binding arm I3RB218 and the CD3 binding arm CD3B219. 8747 is described in WO2016036937A1. Table 13 shows the amino acid sequences of I3RB218. The amino acid sequences of CD3B219 are shown in Table 4.

An exemplary CD123xCD3 bispecific antibody that is used in the experiments is 8747 and comprises the following sequences:
a CD123 binding domain comprising the HCDR9, the HCDR2, the HCDR3, the LCDR, the LCDR2 and the LCDR3 of SEQ ID NOs: 94, 95, 96, 9, 10 and 59, respectively, and a CD3 binding domain comprising the HCDR1, the HCDR2, the HCDR3, the LCDR1, the LCDR2 and
the LCDR3 of SEQ ID NOs: 33, 34, 35, 36, 37 and 38, respectively; the CD123 binding domain comprising the VH of SEQ ID NO: 100 and the VL of SEQ ID NO: 61 and the CD3 binding domain comprises the VH of SEQ ID NO: 39 and the VL of SEQ ID NO: 40; and
a first heavy chain (HC1) of SEQ ID NO: 102, a first light chain (LC1) of SEQ ID NO: 63, a second heavy chain (HC2) of SEQ ID NO: 41 and a second light chain (LC2) of SEQ ID NO: 42.

The anti-CD123 xCD3 bispecific antibody is an IgG4 isotype.

The HC1 comprises S228P, F234A and L235A substitutions.

The HC2 comprises S228P, F234A, L235A, F405L and R409K substitutions.

TABLE 13

| | Region | Sequence | SEQ ID NO: |
|---|---|---|---|
| I3RB218 | HCDR1 | GYWMH | 94 |
| | HCDR2 | AIRSDGSSKYYADSVKG | 95 |
| | HCDR3 | DGVIEDTFDY | 96 |
| | LCDR1 | RASQSVSSYLA | 9 |
| | LCDR2 | DASNRAT | 10 |
| | LCDR3 | QQRSNWPLT | 59 |
| | VH | EVQLLESGGGLVQPGGSLRLSCAASGFTFSGYW | 100 |
| | | MHWVRQAPGKGLEWVSAIRSDGSSKYYADSVK | |
| | | GRFTISRDNSKNTLYLQMNSLRAEDTAVYYCAK | |
| | | DGVIEDTFDYWGQGTLVTVSS | |
| | VL | EIVLTQSPATLSLSPGERATLSCRASQSVSSYLAW | 61 |
| | | YQQKPGQAPRLLIYDASNRATGIPARFSGSGSGT | |
| | | DFTLTISSLEPEDFAVYYCQQRSNWPLTFGQGTK | |
| | | VEIK | |
| | HC | EVQLLESGGGLVQPGGSLRLSCAASGFTFSGYW | 102 |
| | | MHWVRQAPGKGLEWVSAIRSDGSSKYYADSVK | |
| | | GRFTISRDNSKNTLYLQMNSLRAEDTAVYYCAK | |
| | | DGVIEDTFDYWGQGTLVTVSSASTKGPSVFPLAP | |
| | | CSRSTSESTAALGCLVKDYFPEPVTVSWNSGALT | |
| | | SGVHTFPAVLQSSGLYSLSSVVTVPSSSLGTKTYT | |
| | | CNVDHKPSNTKVDKRVESKYGPPCPPCPAPEAA | |
| | | GGPSVFLFPPKPKDTLMISRTPEVTCVVVDVSQE | |
| | | DPEVQFNWYVDGVEVHNAKTKPREEQFNSTYR | |
| | | VVSVLTVLHQDWLNGKEYKCKVSNKGLPSSIEK | |
| | | TISKAKGQPREPQVYTLPPSQEEMTKNQVSLTCL | |
| | | VKGFYPSDIAVEWESNGQPENNYKTTPPVLDSD | |
| | | GSFFLYSRLTVDKSRWQEGNVFSCSVMHEALHN | |
| | | HYTQKSLSLSLGK | |
| | LC | EIVLTQSPATLSLSPGERATLSCRASQSVSSYLAW | 63 |
| | | YQQKPGQAPRLLIYDASNRATGIPARFSGSGSGT | |
| | | DFTLTISSLEPEDFAVYYCQQRSNWPLTFGQGTK | |
| | | VEIKRTVAAPSVFIFPPSDEQLKSGTASVVCLLNN | |
| | | FYPREAKVQWKVDNALQSGNSQESVTEQDSKDS | |
| | | TYSLSSTLTLSKADYEKHKVYACEVTHQGLSSPV | |
| | | TKSFNRGEC | |

To assess effect of pre-treatment with anti-CD38 antibodies on efficacy of tumor killing by the T cell redirecting therapeutics, tumor cells are isolated from subjects having tumors expressing the antigen the T-cell redirecting therapeutic binds, such as CD123, CD33, PSMA, TMEFF2 and the like, or established tumor cell lines are used. Tumor cell killing is assessed ex vivo by co-incubating tumor cells with PB-MNCs obtained from the anti-CD38 antibody exposed or anti-CD38 antibody naïve subjects as described in the Examples, and percentage of lysis of tumor cells is assessed in each group In a separate example, T-cell redirecting therapeutic and the anti-CD38 antibody are incubated together or individually with target and effector cells and the tumor cell killing mediated by the combination vs. individual therapeutics is assessed.

The effect of the anti-CD38 antibody on CD123xCD3 bispecific antibody-mediated tumor cell killing is assessed using CD123 positive tumor cells such as AML tumors, or cell lines such as AML cell lines KG1a, HL60 or MOLM13 as target cells.

The effect of the anti-CD38 antibody on CD33xCD3 bispecific antibody-mediated tumor cell killing is assessed using CD33 positive tumor cells such as AML tumors, or cell lines such as AML cell lines KG1a, HL60 or MOLM13 as target cells.

The effect of the anti-CD38 antibody on TMEFF2xCD3 bispecific antibody-mediated tumor cell killing is assessed using TMEFF2 positive tumor cells such as LnCP cells as target cells.

The effect of the anti-CD38 antibody on PSMAxCD3 bispecific antibody-mediated tumor cell killing is assessed using TMEFF2 positive tumor cells such as LnCP cells as target cells.

PBMCs or BM-MNCs isolated from subjects who have received the anti-CD38 antibody or who are naïve to anti-CD38 antibody treatment are used as effector cells.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

1-212. (canceled)

213. A method of treating a cancer in a subject, comprising administering a therapeutically effective amount of a T-cell redirecting therapeutic that binds GPRC5D and an anti-CD38 antibody to the subject to treat the cancer.

214. The method of claim 213, wherein the anti-CD38 antibody is administered to subject prior to administering the T cell redirecting therapeutic that binds GPRC5D.

215. The method of claim 213, wherein the subject is relapsed or refractory to treatment with a prior anti-cancer therapeutic.

216. The method of claim 213, wherein the cancer is a GPRC5D-expressing cancer.

217. The method of claim 216, wherein the GPRC5D-expressing cancer is a hematological malignancy or a solid tumor.

218. The method of claim 217, wherein the hematological malignancy is a leukemia, a lymphoma, or a multiple myeloma.

219. The method of claim 218, wherein the solid tumor is an ovarian cancer, a lung cancer, a stomach cancer, a prostate cancer, a renal carcinoma, a liver cancer, a pancreatic cancer, a colon cancer, an oesophageal cancer, a bladder cancer, a cervical carcinoma or a malignant melanoma.

220. The method of claim 213, wherein the subject is relapsed or refractory to treatment with the anti-CD38 antibody, lenalinomide, bortezomib, pomalidomide, carfilzomib, elotozumab, ixazomib, melphalan or thalidomide, or any combination thereof.

221. The method of claim 220, wherein the subject is relapsed or refractory to treatment with the anti-CD38 antibody.

222. The method of claim 218, wherein the multiple myeloma is a newly diagnosed multiple myeloma.

223. The method of claim 218, wherein the multiple myeloma is a relapsed or refractory multiple myeloma.

224. The method of claim 218, wherein the multiple myeloma is a high-risk multiple myeloma.

225. The method of claim 224, wherein the subject having the high-risk multiple myeloma has one or more chromosomal abnormalities comprising:
(a) t(4; 14)(p16; q32);
(b) t(14; 16)(q32; q23);
(c) del17p;
(d) 1qAmp;
(e) t(4; 14)(p16; q32) and t(14; 16)(q32; q23);
(f) t(4; 14)(p16; q32) and del17p;
(g) t(14; 16)(q32; q23) and del17p; or
(h) t(4; 14)(p16; q32), t(14; 16)(q32; q23) and del17p, or any combination thereof.

226. The method of claim 213, wherein the T-cell redirecting therapeutic binds CD3, CD3 epsilon (CD3ε), CD8, KI2L4, NKG2E, NKG2D, NKG2F, BTNL3, CD186, BTNL8, PD-1, CD195, or NKG2C.

227. The method of claim 213, wherein the T-cell redirecting therapeutic comprises a GPRC5D binding domain comprising a heavy chain complementarity determining region 1 (HCDR1) of SEQ ID NO: 43, an HCDR2 of SEQ ID NO: 44, an HCDR3 of SEQ ID NO: 45, a light chain complementarity determining region 1 (LCDR1) of SEQ ID NO: 46, an LCDR2 of SEQ ID NO: 47 and an LCDR3 of SEQ ID NO: 48, and a CD3 binding domain comprising an HCDR1 of SEQ ID NO: 33, an HCDR2 of SEQ ID NO: 34, an HCDR3 of SEQ ID NO: 35, an LCDR1 of SEQ ID NO: 36, an LCDR2 of SEQ ID NO: 37 and an LCDR3 of SEQ ID NO: 38.

228. The method of claim 213, wherein the GPRC5D binding domain comprises a heavy chain variable region (VH) of SEQ ID NO: 49 and a light chain variable region (VL) of SEQ ID NO: 50 and the CD3 binding domain comprises a VH of SEQ ID NO: 39 and a VL of SEQ ID NO: 40.

229. The method of claim 213, wherein the T-cell redirecting therapeutic that binds GPRC5C is a multispecific antibody, a CAR or a T cell expressing the CAR.

230. The method of claim 229, wherein the multispecific antibody is an IgG1, an IgG2, an IgG3 or an IgG4 isotype.

231. The method of claim 230, wherein the multispecific antibody comprises one or more Fc substitutions that reduces binding of the multispecific antibody to an Fcγ receptor (FcγR).

232. The method of claim 231, wherein the one or more Fc substitutions are selected from the group consisting of F234A/L235A on IgG4, L234A/L235A on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4 and S228P/F234A/L235A/G236-deleted/G237A/P238S on IgG4, wherein residue numbering is according to the EU index.

233. The method of claim 232, wherein the multispecific antibody further comprises an S228P substitution.

234. The method of claim 229, wherein the multispecific antibody comprises one or more asymmetric substitutions in a first CH3 domain or in a second CH3 domain, or in both the first CH3 domain and the second CH3 domain.

235. The method of claim 234, wherein the one or more asymmetric substitutions are selected from the group consisting of F450L/K409R, wild-type/F409L_R409K, T366Y/F405A, T366W/F405W, F405W/Y407A, T394W/Y407T, T394S/Y407A, T366W/T394S, F405W/T394S and T366W/T366S_L368A_Y407V, L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F and T350V_L351Y_F405A_Y407V/T350V_T366L_K392L_T394W.

236. The method of claim 213, wherein the multispecific antibody comprises the HC1 of SEQ ID NO: 51, the LC1 of SEQ ID NO: 52, the HC2 of SEQ ID NO: 41 and the LC2 of SEQ ID NO: 42.

237. The method of claim 213, wherein the anti-CD38 antibody comprises an HCDR1 of SEQ ID NO: 6, an HCDR2 of SEQ ID NO: 7, an HCDR3 of SEQ ID NO: 8, an LCDR1 of SEQ ID NO: 9, an LCDR2 of SEQ ID NO: 10 and an LCDR3 of SEQ ID NO: 11.

238. The method of claim 213, wherein the anti-CD38 antibody comprises a VH of SEQ ID NO: 4 and a VL of SEQ ID NO: 5.

239. The method of claim 213, wherein the anti-CD38 antibody is an IgG1 isotype.

240. The method of claim 213, wherein the anti-CD38 antibody comprises a heavy chain (HC) of SEQ ID NO: 12 and a light chain (LC) of SEQ ID NO: 13.

241. The method of claim 213, wherein the anti-CD38 antibody comprises:
(a) a VH of SEQ ID NO: 14 and a VL of SEQ ID NO: 15;
(b) a VH of SEQ ID NO: 16 and a VL of SEQ ID NO: 17;
(c) a VH of SEQ ID NO: 18 and a VL of SEQ ID NO: 19; or
(d) a VH of SEQ ID NO: 20 and a VL of SEQ ID NO: 21.

242. The method of claim 241, wherein the anti-CD38 antibody is an IgG1 isotype.

243. The method of claim 213, wherein the anti-CD38 antibody is administered at a dose of between about 8 mg/kg and about 16 mg/kg.

244. The method of claim 213, wherein the T-cell redirecting therapeutic that binds GPRC5D and the anti-CD38 antibody are administered by an intravenous injection.

245. The method of claim 213, wherein the T-cell redirecting therapeutic that binds GPRC5D is administered by an intravenous injection and the anti-CD38 antibody is administered by a subcutaneous injection.

246. The method of claim 213, wherein the subject is a human.

247. The method of claim 213, wherein the T cell redirecting therapeutic that binds GPRC5D is a GPRC5DxCD3 bispecific antibody.

248. The method of claim 213, further comprising administering to the subject one or more anti-cancer therapies.

249. The method of claim 248, wherein the one or more anti-cancer therapies are selected from the group consisting of an autologous stem cell transplant (ASCT), radiation, surgery, a chemotherapeutic agent, an immunomodulatory agent and a targeted cancer therapy.

250. The method of claim 248, wherein the one or more anti-cancer therapies are selected from the group consisting of lenalidomide, thalidomide, pomalidomide, bortezomib, carfilzomib, elotozumab, ixazomib, melphalan, dexamethasone or prednisone.

251. The method of claim 213, wherein the anti-CD38 antibody is administered or provided for administration in a pharmaceutical composition comprising between about 20 mg/mL to about 120 mg/mL of the anti-CD38 antibody in about 25 mM acetic acid, about 60 mM sodium chloride, about 140 mannitol and about 0.04% w/v polysorbate-20 (PS-20); at pH about 5.5.

252. The method of claim 213, wherein the anti-CD38 antibody is administered or provided for administration in a pharmaceutical composition comprising about 1,800 mg of the anti-CD38 antibody and about 30,000 U of rHuPH20.

253. The method of claim 252, wherein the anti-CD38 antibody is administered or provided for administration in a pharmaceutical composition comprising about 120 mg/mL of the anti-CD38 antibody and about 2,000 U/mL of rHuPH20.

254. The method of claim 253, wherein the anti-CD38 antibody is administered or provided for administration in a pharmaceutical composition comprising:
(a) between about 100 mg/mL and about 120 mg/mL of the anti-CD38 antibody;
(b) between about 5 mM and about 15 mM histidine;
(c) between about 100 mM and about 300 mM sorbitol;
(d) between about 0.01% w/v and about 0.04% w/v PS-20; and
(e) between about 1 mg/mL and about 2 mg/mL methionine, at a pH of about 5.5-5.6.

255. The method of claim 254, wherein the anti-CD38 antibody is administered or provided for administration in a pharmaceutical composition comprising:
(a) about 1,800 mg of the anti-CD38 antibody;
(b) about 30,000 U of rHuPH20;
(c) about 10 mM histidine;
(d) about 300 mM sorbitol;
(e) about 0.04% (w/v) PS-20; and
(f) about 1 mg/mL methionine, at a pH of about 5.6.

256. The method of claim 255, wherein the anti-CD38 antibody is administered or provided for administration in a pharmaceutical composition comprising:
(a) about 120 mg/mL of the anti-CD38 antibody;
(b) about 2,000 U/mL of rHuPH20;
(c) about 10 mM histidine;
(d) about 300 mM sorbitol;
(e) about 0.04% (w/v) PS-20; and
(f) about 1 mg/mL methionine, at a pH of about 5.6.

257. A pharmaceutical combination comprising a GPRC5DxCD3 bispecific antibody comprising a GPRC5D binding domain comprising an HCDR1 of SEQ ID NO: 43, an HCDR2 of SEQ ID NO: 44, an HCDR3 of SEQ ID NO: 45, an LCDR1 of SEQ ID NO: 46, an LCDR2 of SEQ ID NO: 47 and an LCDR3 of SEQ ID NO: 48, and a CD3 binding domain comprising an HCDR1 of SEQ ID NO: 33, an HCDR2 of SEQ ID NO: 34, an HCDR3 of SEQ ID NO: 35, an LCDR1 of SEQ ID NO: 36, an LCDR2 of SEQ ID NO: 37 and an LCDR3 of SEQ ID NO: 38 and an anti-CD38 antibody comprising an HCDR1 of SEQ ID NO: 6, an HCDR2 of SEQ ID NO: 7, an HCDR3 of SEQ ID NO: 8, an LCDR1 of SEQ ID NO: 9, an LCDR2 of SEQ ID NO: 10 and an LCDR3 of SEQ ID NO: 11.

258. The pharmaceutical combination of claim 257, wherein the GPRC5D binding domain comprises a VH of SEQ ID NO: 49 and a VL of SEQ ID NO: 50 and the CD3 binding domain comprises a VH of SEQ ID NO: 39 and a VL of SEQ ID NO: 40, and the anti-CD38 antibody comprises a VH of SEQ ID NO: 4 and a VL of SEQ ID NO: 5.

259. The pharmaceutical combination of claim 258, wherein the GPRC5CxCD3 bispecific antibody comprises a first heavy chain (HC1) of SEQ ID NO: 51, a first light chain (LC1) of SEQ ID NO: 52, a second heavy chain (HC2) of SEQ ID NO: 41 and a second light chain (LC2) of SEQ ID NO: 42, and the anti-CD38 antibody comprises an HC of SEQ ID NO: 12 and an LC of SEQ ID NO: 13.

260. The pharmaceutical combination of claim 257, which is a non-fixed combination.

261. The pharmaceutical combination of claim 260, comprising from about 20 mg/mL to about 120 mg/mL of the anti-CD38 antibody in about 25 mM acetic acid, about 60 mM sodium chloride, about 140 mannitol and about 0.04% w/v polysorbate-20 (PS-20); at pH about 5.5.

262. The pharmaceutical combination of claim 260, comprising about 1,800 mg of the anti-CD38 antibody and about 30,000 U of rHuPH20.

263. The pharmaceutical composition of claim 261, comprising about 120 mg/mL of the anti-CD38 antibody and about 2,000 U/mL of rHuPH20.

264. The pharmaceutical combination of claim 263, further comprising one or more excipients.

265. The pharmaceutical combination of claim 264, wherein the one or more excipients are histidine, methionine, sorbitol or polysorbate-20 (PS-20), or any combination thereof.

266. The pharmaceutical combination of claim 265, wherein the pharmaceutical composition comprises:
(a) between about 100 mg/mL and about 120 mg/mL of the anti-CD38 antibody;
(b) between about 5 mM and about 15 mM histidine;
(c) between about 100 mM and about 300 mM sorbitol;
(d) between about 0.01% w/v and about 0.04% w/v PS-20; and
(e) between about 1 mg/mL and about 2 mg/mL methionine, at a pH of about 5.5-5.6.

267. The pharmaceutical combination of claim 266, comprising about 10 mM histidine.

268. The pharmaceutical combination of claim 266, comprising about 300 mM sorbitol.

269. The pharmaceutical combination of claim 265, comprising about 0.04% (w/v) PS-20.

270. The pharmaceutical combination of claim 265, comprising about 1 mg/mL methionine.

271. The pharmaceutical combination of claim 265, comprising:
(a) about 1,800 mg of the anti-CD38 antibody;
(b) about 30,000 U of rHuPH20;
(c) about 10 mM histidine;
(d) about 300 mM sorbitol;
(e) about 0.04% (w/v) PS-20; and
(f) about 1 mg/mL methionine, at a pH of about 5.6.

272. The pharmaceutical combination of claim 266, comprising:
(a) about 120 mg/mL of the anti-CD38 antibody;
(b) about 2,000 U/mL of rHuPH20;
(c) about 10 mM histidine;
(d) about 300 mM sorbitol;
(e) about 0.04% (w/v) PS-20; and
(f) about 1 mg/mL methionine, at a pH of about 5.6.

273. A kit comprising the pharmaceutical combination of claim 257.
